SEQUENCE LISTING (13 sequences)

SEQ ID NO: 1 - DNA, 16 bp, synthetic construct
  source 1..16: mol_type = other DNA
ggataatggttggtca

SEQ ID NO: 2 - DNA, 16 bp, synthetic construct
  source 1..16: mol_type = other DNA
  modified_base 1..3: mod_base = OTHER; note = beta-D-oxy LNA nucleotide
  modified_base 15..16: mod_base = OTHER; note = beta-D-oxy LNA nucleotide
caagtaagcgtgtagc

SEQ ID NO: 3 - DNA, 16 bp, synthetic construct
  source 1..16: mol_type = other DNA
  modified_base 1..3: mod_base = OTHER; note = beta-D-oxy LNA nucleotide
  modified_base 15..16: mod_base = OTHER; note = beta-D-oxy LNA nucleotide
gtctggtaactatgag

SEQ ID NO: 4 - DNA, 16 bp, synthetic construct
  source 1..16: mol_type = other DNA
  modified_base 1..3: mod_base = OTHER; note = beta-D-oxy LNA nucleotide
  modified_base 15..16: mod_base = OTHER; note = beta-D-oxy LNA nucleotide
agattcatgcagatgg

SEQ ID NO: 5 - DNA, 16 bp, synthetic construct
  source 1..16: mol_type = other DNA
  modified_base 1..3: mod_base = OTHER; note = beta-D-oxy LNA nucleotide
  modified_base 15..16: mod_base = OTHER; note = beta-D-oxy LNA nucleotide
atgatgtgcaacaggc

SEQ ID NO: 6 - DNA, 16 bp, synthetic construct
  source 1..16: mol_type = other DNA
  modified_base 1..3: mod_base = OTHER; note = beta-D-oxy LNA nucleotide
  modified_base 15..16: mod_base = OTHER; note = beta-D-oxy LNA nucleotide
ccctgtattaccctgc

SEQ ID NO: 7 - DNA, 18 bp, synthetic construct
  source 1..18: mol_type = other DNA
cacgtgagcatactgggc

SEQ ID NO: 8 - DNA, 20 bp, synthetic construct
  source 1..20: mol_type = other DNA
gcagagtcatcaaagggtcg

SEQ ID NO: 9 - DNA, 21 bp, synthetic construct
  source 1..21: mol_type = other DNA
gtgctacacagaagtggattc

SEQ ID NO: 10 - DNA, 20 bp, synthetic construct
  source 1..20: mol_type = other DNA
cctcagtcctagcttcatca

SEQ ID NO: 11 - DNA, 16 bp, synthetic construct
  source 1..16: mol_type = other DNA
  modified_base 1..3: mod_base = OTHER; note = beta-D-oxy LNA nucleotide
  modified_base 15..16: mod_base = OTHER; note = beta-D-oxy LNA nucleotide
ggataatggttggtca

SEQ ID NO: 12 - DNA, 20 bp, synthetic construct
  source 1..20: mol_type = other DNA
agaacttgaagctgtcaggg

SEQ ID NO: 13 - DNA, 20 bp, synthetic construct
  source 1..20: mol_type = other DNA
tgcatgttgaagacagcact

(19) United States
(12) Patent Application Publication
(10) Pub. No.: US 2025/0051776 A1
(43) Pub. Date: Feb. 13, 2025

(54) COMPOSITIONS AND METHODS OF MODULATING LONG NONCODING TRANSCRIPTS ASSOCIATED WITH ARDS INDUCED PULMONARY FIBROSIS

(71) Applicant: HAYA Therapeutics SA, Epalinges (CH)

(72) Inventors: Samir OUNZAIN, Lausanne (CH); Alexandra IOURANOVA, Chavannes-près-Renens (CH); Daniel Roman BLESSING, Saint-Sulpice (CH)

(21) Appl. No.: 18/927,263

(22) Filed: Oct. 25, 2024

Related U.S. Application Data
(63) Continuation of application No. PCT/IB2023/000233, filed on Apr. 28, 2023.
(60) Provisional application No. 63/336,648, filed on Apr. 29, 2022.

(51) Int. Cl. C12N 15/113; A61P 11/00

(52) U.S. Cl. CPC C12N 15/113; A61P 11/00; C12N 2310/113; C12N 2310/31; C12N 2310/3231; C12N 2310/341

(57) ABSTRACT

Provided herein are modulators of a long noncoding transcript, pharmaceutical compositions comprising the modulator, and kits comprising the modulator. Also provided herein are methods of modulating a long noncoding transcript in a subject in need thereof. Further provided here are methods of preventing, alleviating, or treating pulmonary fibrosis or inflammation induced or associated with ARDS in a subject in need thereof.

### CROSS-REFERENCE

This application is a continuation of International Application No. PCT/IB2023/000233, filed Apr. 28, 2023, which claims the benefit of U.S. Provisional Application No. 63/336,648 filed Apr. 29, 2022, which are incorporated herein by reference in their entireties.

### SEQUENCE LISTINGS

The instant application contains a Sequence Listing which has been submitted electronically in XML file format and is hereby incorporated by reference in its entirety. Said XML copy, created on Oct. 25, 2024, is named 60448-701_301_SL.xml and is 19,152 bytes in size.

### BACKGROUND OF THE DISCLOSURE

Only a small portion of mammalian genome is transcribed to protein-coding mRNAs, with the rest is transcribed to non-coding RNAs. Among them, long non-coding RNAs (lncRNA) are a type of RNA with usually more than 200 nucleotides that are not translated into protein. LncRNAs have been shown to regulate gene expression networks at different levels via various mechanisms (see, e.g., Yao et al., Nature Cell Biology, 21, pages 542-551, 2019).

Acute respiratory distress syndrome (ARDS) occurs when fluid builds up in the air sacs (alveoli) of lungs. It can occur in any individuals who are critically ill or who have significant injuries, and it is often fatal. It is estimated that ARDS affects more than 190,000 people in the USA alone annually, with a mortality of 27-45% (see, e.g., Burnham et al., Eur Respir J. 43(1): 276-285, 2014). Patients with ARDS have severe shortness of breath and often are unable to breathe on their own without support from a ventilator. Patients with ARDS often have reduced oxygenation, extensive alveolar damage, lung inflammation, and lung fibrosis.

There is no fundamental treatment for ARDS at this time, and treatments in the clinic, such as oxygen and fluid management, focus on supporting the patient while the lungs heal. Therefore, there exists an urgent need to develop related therapeutics to prevent, alleviate, and treat ARDS and fibrosis and inflammation that accompanied ARDS.

### SUMMARY OF THE DISCLOSURE

In one aspect, to address the need to prevent, alleviate, and treat ARDS and fibrosis and inflammation that accompanied ARDS, provided herein is a modulator of a long noncoding transcript that is associated with initiation, development, or prognosis of ARDS, or pulmonary fibrosis associated with ARDS. In some aspects, the long-noncoding transcript is transcribed from a genomic region located within chr3:45,806,503 to chr3:45,831,916. Also provided herein is a modulator of a long noncoding transcript, which is transcribed from a genomic region located within chr3:45,806,503 to chr3:45,831,916, and the long noncoding transcript is transcribed using Crick Strand as template.

In some instances, the genomic region is LOC107986083 (chr3: 45,817,379-45,827,511). In other instances, the long noncoding transcript comprises at least a portion of XR_001740681.1 (NCBI), locus ENSG00000288720 (Gencode/ENSEMBL), transcript ENST00000682011.1 (Gencode/ENSEMBL), transcript ENST00000684202.1 (Gencode/ENSEMBL), or transcript RP11-852E15.3 (Gencode).

In some instances, the expression of the long noncoding transcript is elevated in a subject affected by pulmonary fibrosis associated with acute respiratory distress syndrome (ARDS). In other instances, the elevated expression of the long-noncoding transcript is associated with severity of an ARDS symptom in the subject. In other instances, the long noncoding transcript comprises a single nucleotide polymorphism associated with the ARDS.

In some instances, the modulator modifies an expression level and/or an activity of the long noncoding transcript. In some instances, the modulator reduces the elevated expression level and/or activity of long noncoding transcript by at least 50%, at least 60%, at least 70%, at least 80%, at least 90% in a cell or a tissue of the subject, wherein the elevated expression level is associated with initiation, development, or prognosis of ARDS or initiation, development, or prognosis of pulmonary fibrosis associated with ARDS. In other instances, the modulator reduces the elevated expression level and/or activity of the long noncoding transcript to a baseline level.

In some instances, the modulator is a nucleic acid editing or modifying moiety. In some instances, the nucleic acid editing or modifying moiety targets the genomic region, the long noncoding transcript, or a premature form thereof. In some instances, the nucleic acid editing or modifying moiety is a CRISPR-based moiety, a meganuclease-based moiety, a zinc finger nuclease (ZFN)-based moiety, or a transcription activator-like effector-based nuclease (TALEN)-based moiety.

In some instances, the modulator is a synthetic or artificial oligonucleotide or polynucleotide. In some instances, the synthetic or artificial oligonucleotide comprises a nucleic acid sequence complementary to at least 10, 11, 12, 13, 14, or 15 nucleotides of the long noncoding transcript. In some instances, the synthetic or artificial oligonucleotide or polynucleotide is a small interfering RNA (siRNA), a microRNA (miRNA), an inhibitory double stranded RNA (dsRNA), a small or short hairpin RNA (shRNA), an antisense oligonucleotide (ASO), a piwi-interacting RNA (piRNA), a heterogeneous nuclear RNA (hnRNA), a small nuclear RNA (snRNA), or an enzymatically-prepared siRNA (esiRNA) or the precursors thereof.

In some instances, the synthetic oligonucleotide is about 10-50 nucleotides long, about 10-30 nucleotides long, or about 14-20 nucleotides long. In some instances, the synthetic oligonucleotide is about 16 nucleotides long.

In some instances, the synthetic oligonucleotide comprises one or more sugar modifications, one or more phosphate modifications, one or more base modifications, one or more pyrimidine modifications, or any combination thereof. In some instances, the one or more sugar modifications are locked nucleic acid (LNA), tricyclo-DNA, 2′-fluoro, 2′-O-methyl, 2′-methoxyethyl (2′-MOE), 2′-cyclic ethyl (cET), UNA, and conformationally restricted nucleoside (CRN), or any combination thereof. In some instances, the one or more phosphate modifications comprise phosphorothioate internucleotide linkage, methylphosphonate internucleotide linkage, guanidinopropyl phosphoramidate internucleotide linkage, or any combination thereof. In some instances, the one or more base modifications comprise a purine modifications selected from a group consisting of 2,6-diaminopurin, 3-deaza-adenine, 7-deaza-guanine, 8-zaido-adenine, or any combination thereof. In some instances, the one or more base modifications comprise a pyrimidine modifications selected from a group consisting of 2-thio-thymidine, 5-carboxamide-uracil, 5-methyl-cytosine, 5-ethynyl-uracil, or any combination thereof. In some instances, the synthetic oligonucleotide comprises a phosphorodiamidate morpholino oligomer (PMO).

In some instances, the synthetic oligonucleotide is an ASO, wherein the ASO comprises a gapmer comprising a central region of consecutive DNA nucleotides flanked by a 5′-wing region and 3′-wing region, wherein at least one of 5′-wing region and 3′-wing region comprises a nucleic acid analogue. In some instances, the nucleic acid analogue comprises an LNA. In some instances, the LNA comprises a beta-D-oxy LNA, an alpha-L-oxy-LNA, a beta-D-amino-LNA, an alpha-L-amino-LNA, a beta-D-thio-LNA, an alpha-L-thio-LNA, a 5′-methyl-LNA, a beta-D-ENA, or an alpha-L-ENA. In some instances, the LNA comprises a beta-D-oxy LNA.

In some instances, the 5′-wing region comprises at least two LNAs. In some instances, the 5′-wing region comprises three consecutive LNAs. In some instances, the 3′-wing region comprises an LNA. In some instances, the 3′-wing region comprises two consecutive LNAs.

In some instances, the synthetic oligonucleotide comprises at least 9, 10, 11, 12, 13, 14 consecutive nucleotides with no more than 1, 2, 3 mismatches from any one of SEQ ID NOs: 1-4. In some instances, the synthetic oligonucleotide comprises a nucleic acid sequence at least 80%, at least 85%, at least 90%, at least 95%, at least 96%, at least 97%, at least 98%, at least 99% identical to a sequence selected from any one of SEQ ID NOs: 1-4.

In some aspects, provided herein is a pharmaceutical composition comprising the modulator disclosed herein and a pharmaceutically acceptable salt or derivative thereof. In other aspects, provided herein is a kit comprising the modulator disclosed herein or the pharmaceutical composition disclosed herein.

In some aspects, provided herein is a modulator comprising an antisense oligonucleotide (ASO), wherein the ASO comprises at least 9, 10, 11, 12, 13, 14 consecutive nucleotides with no more than 1, 2, 3 mismatches from SEQ ID NO: 1 (5′-GGATAATGGTTGGTCA-3′). In some instances, the ASO comprises a nucleic acid sequence of 80%, at least 85%, at least 90%, at least 95%, at least 96%, at least 97%, at least 98%, at least 99% identical to SEQ ID NO: 1 (5′-GGATAATGGTTGGTCA-3′).

In some instances, the ASO comprises a gapmer comprising a central region of consecutive DNA nucleotides flanked by a 5′-wing region and 3′-wing region, wherein at least one of 5′-wing region and 3′-wing region comprises a nucleic acid analogue. In some instances, the nucleic acid analogue comprises a locked nucleic acid (LNA). In some instances, the LNA comprises a beta-D-oxy LNA, an alpha-L-oxy-LNA, a beta-D-amino-LNA, an alpha-L-amino-LNA, a beta-D-thio-LNA, an alpha-L-thio-LNA, a 5′-methyl-LNA, a beta-D-ENA, or an alpha-L-ENA.

In some instances, the 5′-wing region comprises at least two LNAs. In some instances, the 5′-wing region comprises three consecutive LNAs. In some instances, the 3′-wing region comprises an LNA. In some instances, the 3′-wing region comprises two consecutive LNAs.

In some aspects, provided herein is a method of modulating a long noncoding transcript in a subject in need thereof, the method comprising administering to the subject the modulator disclosed herein, or a pharmaceutical composition disclosed herein.

In some aspects, provided herein is a method of preventing, alleviating, or treating pulmonary fibrosis in a subject in need thereof, the method comprising administering to the subject an effective amount of the modulator disclosed herein, or a pharmaceutical composition disclosed herein. In some aspects, provided herein is a method of preventing, alleviating, or treating idiopathic pulmonary fibrosis in a subject in need thereof, the method comprising administering to the subject an effective amount of the modulator disclosed herein, or a pharmaceutical composition disclosed herein.

In some instances, the modulator is expressed or encapsulated in a viral or plasmid vector, a liposome, or a nanoparticle. In some instances, the administering is performed intratracheally, orally, nasally, intravenously, intraperitoneally, or intramuscularly. In some instances, the administering is a targeted delivery to a lung tissue of the subject. In some instances, the administering is in a form of aerosol.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings (also “Figure” and “FIG.” herein), of which:

FIG. 1A-FIG. 1B illustrate diagrams of discovery pipelines. FIG. 1A illustrates workflows from data collection to lncRNA candidates filtering. FIG. 1B illustrates pulmonary fibrosis model and possible experiment readouts.

FIG. 2A-FIG. 2C illustrate the genetic characterization of LOC107986083. FIG. 2A shows an annotated characterization of the human genomic region of LOC107986083, illustrating the locations of various CORAL transcripts, nearby genes, and compiled results of genetic and epigenetic characterization of LOC107986083. Relative locations of rs17713054 and ASO target sequences are also shown here. FIG. 2B shows a close-up view of annotated characterization of the human genomic region near parts of the lncRNA (CORAL transcripts), rs17713054, the location of target sequence for ASO-1, and the 3′ end of LZTFL1. Also shown here are compiled results of genetic characterization of LOC107986083, including assay for DNAse-seq, chromatin immunoprecipitation (ChIP)-seq with H3K27ac of regions in the downstream of a protein coding gene LZTFL, ATAC-Seq, snATAC-Seq, and deep RNA-Seq. FIG. 2C shows tissue-specific expression of LOC107986083 and LZTFL1.

FIG. 3A-FIG. 3S illustrate an establishment of an in-vitro model for pulmonary fibrosis. FIG. 3A shows a diagram of procedures. FIG. 3B shows microscopic images of the morphologies of myofibroblast transformation in the in-vitro model. FIG. 3C shows results of PRO-seq and CUT&RUN with various markers for fibronectin 1 (FN1) in response to serum starvation and TGFβ treatment. FIG. 3D shows graphs of CORAL expression in fibroblasts (FB) and myofibroblasts (MyoFB) from various tissues (HLF: human lung fibroblast; HCF: human cardiac fibroblast; HDF: human dermal fibroblast). FIG. 3E shows transcription level of a lncRNA from LOC107986083 (labeled here as CORAL) in response to serum starvation and TGFβ treatment. FIG. 3F shows transcription level of aSMA in response to serum starvation and TGFβ treatment. FIG. 3G shows transcription level of CollA1 in response to serum starvation and TGFβ treatment. FIG. 3H shows transcription level of Col3A1 in response to serum starvation and TGFβ treatment. FIG. 3I shows transcription level of periostin in response to serum starvation and TGFβ treatment. FIG. 3J shows transcription level of fibronectin in response to serum starvation and TGFβ treatment. FIG. 3K shows a graph of aSMA protein expression over 24-96 hr in lung FB and lung MyoFB. FIG. 3L illustrates the subcellular localization of the transcript of LOC107986083 in response to serum starvation and TGFβ treatment. FIG. 3M shows a diagram of a procedure to induce lung myofibrosis in vitro. FIG. 3N shows single-cell sequencing of cells with and without pirfenidone, with and without serum starvation and TGFβ treatment, for different durations. FIG. 3O shows ACTA2, COL3A1, and FAP RNA expression in negative control cells (48 hr, drug(−), TGFβ(−)), TGFβ treated cells (48 hr, drug(−), TGF(+)), and positive control cells (48 hr, drug(+), TGFβ(+)). FIG. 3P shows grouping of different cell subpopulations based on expression in a panel of genes. FIG. 3Q shows Kaminski et al. labels transferred to the internal single cell atlas. FIG. 3R shows Banovich et al. labels transferred to the internal single cell atlas. FIG. 3S shows RNA expression of ACTA2, COL3A1, POSTN, FAP, PDGFRB, and SMAD3 among three different cell types (myofibroblasts, proliferating epithelial cells, and proliferating macrophages).

FIG. 4A-FIG. 4AG illustrate validation of ASOs against the transcript of human LOC107986083. FIG. 4A shows a diagram of experiment procedure of the validation. FIG. 4B shows the successful knock-down of the elevated transcript of human LOC107986083 in response to serum starvation and TGFβ treatment with an ASO with the sequence of SEQ ID NO: 1 as assayed by qPCR. FIG. 4C shows the successful knock-down of the elevated expression of ACTA2 in response to serum starvation and TGFβ treatment with an ASO with the sequence of SEQ ID NO: 1 as assayed by qPCR. FIG. 4D shows the successful knock-down of the elevated expression of CollA1 in response to serum starvation and TGFβ treatment with an ASO with the sequence of SEQ ID NO: 1 as assayed by qPCR. FIG. 4E shows the successful knock-down of the elevated expression of Col3A1 in response to serum starvation and TGFβ treatment with an ASO with the sequence of SEQ ID NO: 1 as assayed by qPCR. FIG. 4F shows the modulation of the elevated expression of FAP in response to serum starvation and TGFβ treatment with an ASO with the sequence of SEQ ID NO: 1 as assayed by qPCR. FIG. 4G shows the successful knock-down of the elevated expression of Fibronectin 1 in response to serum starvation and TGFβ treatment with an ASO with the sequence of SEQ ID NO: 1 as assayed by qPCR. FIG. 4H shows the successful knock-down of the elevated expression of POSTN in response to serum starvation and TGFβ treatment with an ASO with the sequence of SEQ ID NO: 1 as assayed by qPCR. FIG. 4I shows significant effects of ASO-1 treatment to reduce expression of selected markers of fibrosis (ACTA2, COL1A1, COL3A1, FAP, FN1, and POSTN) by RNA-Seq analysis in response to serum starvation and TGFβ treatment in the in-vitro model for pulmonary fibrosis used in this example. FIG. 4J shows the results from Uniform Manifold Approximation and Projection (UMAP) analysis demonstrating that FB cells separate from MyoFB cells and MyoFB-ASO_Scr-treated cells, confirming a difference in cell states between FB and MyoFB and results from cell cycle analysis between cell states. FIG. 4K shows results from UMAP of MyoFB-ASO_Scr-treated cells compared to MyoFB-ASO-1-treated cells demonstrating that ASO-1 treatment induces a distinction detection in cell classification and produces a slight increase in the proportion of proliferating to quiescent cells. FIG. 4L shows graphs examining expression of select fibrosis-related genes in FB cells relative to MyoFB cells in the upper row and MyoFB-ASO_Scr-treated cells relative to MyoFB-ASO-1-treated cells in the lower row. FIG. 4M shows the results of UMAP MyoFB-ASO_Scr-treated cells compared to MyoFB-ASO-1-treated cells for select fibrosis markers. FIG. 4N shows the results of a Gene Set Enrichment Analysis (GSEA) study examining changes in expression of MyoFB-ASO_Scr-treated cells to MyoFB-ASO-1-treated cells using snRNA-Seq and demonstrates downregulation of 398 genes and upregulation of 266 genes in MyoFB-ASO-1-treated cells. Gene ontology terms for genes going up after transfection of an ASO with the sequence of SEQ ID NO: 1 are shown and gene ontology terms for genes going down after transfection of an ASO with the sequence of SEQ ID NO: 1 are shown. FIG. 4O shows the effects of various ASOs on modulating elevated transcription of human LOC107986083 in response to serum starvation and TGFβ treatment. MyoFB GI (ASO-2) is an ASO corresponding to SEQ ID NO: 2. MyoFB G2 (ASO-1) is an ASO corresponding to SEQ ID NO: 1. MyoFB G4 (ASO-3) is an ASO corresponding to SEQ ID NO: 3. MyoFB G5 (ASO-4) is an ASO corresponding to SEQ ID NO: 4. ASO-1 significantly knocked down a level of expression of human LOC107986083 in response to serum starvation and TGFβ treatment. ASO 2-4 were not demonstrated to significantly knockdown a level of expression of human LOC107986083 in response to serum starvation and TGFβ treatment. FIG. 4P shows the modulation of the elevated expression of human COL1A1 in response to serum starvation and TGFβ treatment with ASO1-ASO4 in comparison to a negative control scrambled ASO (MyoFB ASO-Scr). FIG. 4Q shows the modulation of the elevated expression of human FAP in response to serum starvation and TGFβ treatment with ASO1-ASO4 in comparison to a negative control scrambled ASO (MyoFB ASO-Scr). FIG. 4R shows the modulation of the elevated expression of human FN1 in response to serum starvation and TGFβ treatment with ASO1-AS04 in comparison to a negative control scrambled ASO (MyoFB ASO-Scr). FIG. 4S shows the modulation of the elevated expression of human POSTN in response to serum starvation and TGFβ treatment with ASO1-ASO4 in comparison to a negative control scrambled ASO (MyoFB ASO-Scr). FIG. 4T shows the modulation of the elevated expression of human LTBP2 in response to serum starvation and TGFβ treatment with ASO1-ASO4 in comparison to a negative control scrambled ASO (MyoFB ASO-Scr). FIG. 4U shows the modulation of the elevated expression of human THBS2 in response to serum starvation and TGFβ treatment with ASO1-ASO4 in comparison to a negative control scrambled ASO (MyoFB ASO-Scr). FIG. 4V shows the modulation of the elevated expression of human CTHRC1 in response to serum starvation and TGFβ treatment with ASO1-ASO4 in comparison to a negative control scrambled ASO (MyoFB ASO-Scr). FIG. 4W shows the results of ASO-1 treatment in significantly modulating transcription state of a human IPF gene signature in ASO-1-treated cells compared to ASO_Scr-treated cells. FIG. 4X shows results of a dose-response experiment testing hsCORAL ASO-1 responses by qPCR against human CORAL and against select markers of fibrosis. FIG. 4Y shows dose-responsive fibrotic gene marker expression profiled by RNA-Seq to ASO-1 treatment in the in-vitro model for pulmonary fibrosis used in this example. FIG. 4Z shows a listing of cellular pathways and biological mechanisms in a calculation for an extent of differential gene expression observed by RNA-Seq modulation for genes grouped under each pathway or mechanism in response to ASO-1 treatment in the in-vitro model for pulmonary fibrosis used in this example. Results of GSEA performed on primary NHLF cells with induced fibrosis showing groupings of genes according to gene ontology (GO) terms that were modulated following ASO-1 treatment that were graphed in a latent space and subjected to f cluster analysis of differential gene expression due to ASO treatment. Immune-related GO terms are indicated with (I) and fibrosis-related GO terms are indicated with (F). FIG. 4AA shows the results of clustering analysis of gene expression indicating that ASO-1 treatment in MyoFB downregulates genes categorized in seven distinct clusters. FIG. 4AB to FIG. 4AC show graphs of further gene expression analysis of a short list of immune-related genes identified as being downregulated following ASO-1 treatment. FIG. 4AD to FIG. 4AF show graphs of dose response calculations for a short list of immune-related genes identified as being downregulated following ASO-1 treatment at various concentrations and calculated RC₅₀ values compared to ASO-Scr-treatment. FIG. 4AG shows bright field microscopic images of fibroblasts (FB) and myofibroblasts (MyoFB) growing in vitro and images of serum starved and TGFβ-treated MyoFB that have been treated with ASO-1, ASO-2, ASO-3, ASO-4, or ASO-Scr at 0 hr, 24 hr, and 48 hr to demonstrate no obvious signs of toxicity following ASO treatment.

FIG. 5A-FIG. 5U illustrate the validation of ASOs against the transcript of the mouse homolog of LOC107986083. FIG. 5A shows whole-lung RNA-Seq data using 1-year old mouse lung tissues. FIG. 5B shows the regulation of the expression of mouse homolog of LOC107986083 in response to serum starvation and TGFβ treatment with two ASOs targeting the mouse homolog of LOC107986083 in an immortalized mouse lung fibroblast cell line as assayed by qPCR. FIG. 5C shows the regulation of the expression of Acta2 in response to serum starvation and TGFβ treatment with two ASOs targeting the mouse homolog of LOC107986083 in an immortalized mouse lung fibroblast cell line as assayed by qPCR. FIG. 5D shows the regulation of the expression of Collal in response to serum starvation and TGFβ treatment with two ASOs targeting the mouse homolog of LOC107986083 in an immortalized mouse lung fibroblast cell line as assayed by qPCR. FIG. 5E shows the regulation of the expression of Col3al in response to serum starvation and TGFβ treatment with two ASOs targeting the mouse homolog of LOC107986083 in an immortalized mouse lung fibroblast cell line as assayed by qPCR. FIG. 5F shows the regulation of the expression of Fn1 in response to serum starvation and TGFβ treatment with two ASOs targeting the mouse homolog of LOC107986083 in an immortalized mouse lung fibroblast cell line as assayed by qPCR. FIG. 5G shows the regulation of the expression of Postn in response to serum starvation and TGFβ treatment with two ASOs targeting the mouse homolog of LOC107986083 in an immortalized mouse lung fibroblast cell line as assayed by qPCR. FIG. 5H shows the regulation of the expression of Acta2 in response to serum starvation and TGFβ treatment with two ASOs targeting the mouse homolog of LOC107986083 in primary mouse lung fibroblasts isolated directly from 5 month old mice as assayed by qPCR. FIG. 5I shows the regulation of the expression of Col3al in response to serum starvation and TGFβ treatment with two ASOs targeting the mouse homolog of LOC107986083 in primary mouse lung fibroblasts isolated directly from 5 month old mice as assayed by qPCR. FIG. 5J shows the regulation of the expression of Fn1 in response to serum starvation and TGFβ treatment with two ASOs targeting the mouse homolog of LOC107986083 in primary mouse lung fibroblasts isolated directly from 5 month old mice as assayed by qPCR. FIG. 5K shows the regulation of the expression of the mouse homolog of LOC107986083 in response to serum starvation and TGFβ treatment with two ASOs targeting the mouse homolog of LOC107986083 in primary mouse lung fibroblasts isolated directly from 24 month old mice as assayed by qPCR. FIG. 5L shows the regulation of the expression of Acta2 in response to serum starvation and TGFβ treatment with two ASOs targeting the mouse homolog of LOC107986083 in primary mouse lung fibroblasts isolated directly from 24 month old mice as assayed by qPCR. FIG. 5M shows the regulation of the expression of Col3al in response to serum starvation and TGFβ treatment with two ASOs targeting the mouse homolog of LOC107986083 in primary mouse lung fibroblasts isolated directly from 24 month old mice as assayed by qPCR. FIG. 5N shows the regulation of the expression of Fn1 in response to serum starvation and TGFβ treatment with two ASOs targeting the mouse homolog of LOC107986083 in primary mouse lung fibroblasts isolated directly from 24 month old mice as assayed by qPCR. FIG. 5O shows graphs demonstrating regulation of the expression of fibrosis markers by mmASO-2 targeting the mouse homolog of LOC107986083 in primary lung fibroblast cells from young (5 month old) mice treated by serum starvation and TGFβ treatment as assessed by RNA-Seq. FIG. 5P shows graphs demonstrating regulation of the expression of fibrosis markers by mmASO-2 targeting the mouse homolog of LOC107986083 in primary lung fibroblast cells from old (24 month old) mice treated by serum starvation and TGFβ treatment as assessed by RNA-Seq. FIG. 5Q shows graphs demonstrating regulation of the expression gene from mouse IPF gene signature markers by mmASO-2 targeting the mouse homolog of LOC107986083 in primary lung fibroblast cells. FIG. 5R shows a listing of cellular pathways and biological mechanisms listed by GO term in a calculation for an extent of differential gene expression modulation for genes grouped under each pathway or mechanism in response to mmASO-2 treatment in mouse pulmonary fibroblasts (from 24 month old mice) treated by serum starvation and TGFβ treatment. GSEA terms downregulated by mmASO-2 are shown here and include ECM-related terms. FIG. 5S shows a selected listing of leukocyte migration related GO terms in a calculation for an extent of differential gene expression modulation for genes grouped under each pathway or mechanism in response to mmASO-2 treatment in mouse pulmonary fibroblasts (from 24 month old mice) treated by serum starvation and TGFβ treatment. GSEA terms downregulated by mmASO-2 are shown here and include leukocyte migration-related terms. FIG. 5T shows graphs demonstrating regulation of the expression of immune-related genes by mmASO-2 targeting the mouse homolog of LOC107986083 in primary lung fibroblast cells (from 24 month old mice) treated by serum starvation and TGFβ treatment. FIG. 5U shows graphs demonstrating regulation of the expression of immune-related genes by mmASO-2 targeting the mouse homolog of LOC107986083 in mouse primary lung fibroblast cells treated by serum starvation and TGFβ treatment.

FIG. 6 illustrates a diagram of the experiment procedures of validating ASOs targeting the mouse homolog of LOC107986083 in a pulmonary fibrosis mouse model.

FIG. 7A-FIG. 7I demonstrate the results of treatment in a bleomycin (Bleo)-induced model of pulmonary inflammation and fibrosis using administration of an ASO directed against the mouse homolog of LOC107986083. FIG. 7A shows graphs demonstrating immune cell numbers following bronchial lavage in the bleomycin-treated mouse model and the effects of mmASO-2 treatment on immune cell number. FIG. 7B shows graphs charting mouse weight at various days following treatment and lung-to-body weight ratio in following treatment according to Example 6. Prophylactic mmASO-2 treatment in the mouse model of bleomycin treatment induced significant weight loss and an increase in lung-to-body weight ratio. FIG. 7C shows the results of GSEA on mmASO-2 downregulated genes following RNA-Seq analysis of bulk lung tissue RNA from mmASO-2-treated mice compared to ASO_Scr-treated mice. Analysis indicates an enrichment of GO terms relating to leukocyte migration. FIG. 7D shows select GSEA results on mmASO-2 downregulated genes following RNA-Seq analysis of bulk lung tissue RNA from mmASO-2-treated mice compared to ASO_Scr-treated mice focusing on selected ECM-related GO terms representing groups of genes having differential expression between test groups. FIG. 7E shows graphs charting expression of select immune-related genes (Clu, Dock2, Fer, Gab2, and Lgals9) in the bleomycin-treated mouse model described in Example 6 and changes following mmASO-2 treatment. FIG. 7F shows charts demonstrating the effects of ASO treatment in a human in vitro pulmonary cell assay of human primary lung fibroblasts in which MyoFB state was induced by serum starvation and TGFβ treatment and a mouse in vivo pulmonary cell assay in which test mice were treated with bleomycin in expression of DOCK2/Dock2 and LGALS9/Lgals9. FIG. 7G shows charts demonstrating cytokines that are significantly upregulated or downregulated in response to mmASO-2 treatment in the mouse bleomycin model described in Example 6. FIG. 7H shows charts demonstrating scoring of pulmonary fibrosis [Average Ashcroft Score (H&E) and Increased Collagen Score (MT)] and immune cell infiltration in the lung [Infiltrate, mixed cells (H&E)] and effect of mmASO-2 treatment in the mouse bleomycin model described in Example 6. FIG. 7I shows histopathological representative micrographs of H&E stained lung tissue of four treatment groups (Group 1 to Group 4) in the mouse bleomycin model described in Example 6.

FIG. 8A to FIG. 8E show the results of constructing, validating and further use of a gene signature for idiopathic pulmonary fibrosis (IPF) pathology and severity. FIG. 8A shows a flow chart depicting sources and format of gene expression input data, type of data analysis, and consolidation into knowledge database of the total results of a method of meta-analysis to build a gene signature for idiopathic pulmonary fibrosis (IPF). FIG. 8B shows a compilation of correlation charts of log² fold change in gene expression in assayed genes via bulk RNA-Seq for various databases (derived from cells maintained in vitro and cells obtained from in vivo sources) relating to pulmonary cells and various treatments therein. FIG. 8C shows output of the meta-analysis compiling differentially expressed genes (DEG) data from various RNA-Seq experiments in pulmonary cells under various treatment conditions in a graph of Log² fold change consistency to identify genes downregulated in pulmonary fibroblasts and also identify genes upregulated in myofibroblasts to create an IPF gene signature that is consistent in vitro and in vivo. FIG. 8D shows a graph of the results from testing ASO-1 treatment on the effect of expression of an unbiased IPF gene set derived from the human IPF gene signature. FIG. 8E shows graphs of human IPF gene signature data derived from four public RNA-Seq datasets and three in house datasets produced under various test conditions.

### DETAILED DESCRIPTION

The present disclosure includes that long noncoding transcripts (e.g., lncRNA) that are associated with onset, development or prognosis of acute respiratory distress syndrome (ARDS) are identified, and that differential expression or transcriptional regulation of the long noncoding transcripts are associated with symptoms of with onset, development or prognosis of ARDS. In some aspects, the long noncoding transcripts are associated with onset or development of pulmonary fibrosis that is associated with or induced by ARDS or a symptom of ARDS. Such long noncoding transcripts could be a druggable target to prevent, alleviate, or treat ARDS or pulmonary fibrosis associated with onset, development or prognosis of, or induced by. As such, in one aspect, provided herein, are modulators of a long noncoding transcript associated with onset, development or prognosis of ARDS, or associated with onset or development of pulmonary fibrosis associated with or induced by ARDS or a symptom of ARDS. Also provided herein includes pharmaceutical compositions comprising the modulator, and kits comprising the modulator. Also provided herein are methods of modulating a long noncoding transcript associated with onset, development or prognosis of ARDS or associated with onset or development of pulmonary fibrosis that is associated with or induced by ARDS or a symptom of ARDS in a subject in need thereof. Further provided herein are methods of preventing, alleviating, or treating ARDS or preventing, alleviating, or treating pulmonary fibrosis or inflammation in a subject affected by ARDS.

Long noncoding transcripts (lncRNAs) Associated with ARDS Induced Pulmonary Fibrosis

ARDS, which occurs when liquid builds up in lungs, is caused by or associated with viral infection, for example, infection of pulmonary disease-associated virus (e.g., COVID-19) or human immunodeficiency virus (HIV), and/or caused by or associated with increased immune response (e.g., autoimmune disease). Particularly, it has been reported that ARDS and lung failure are the main lung diseases in COVID-19 patients, and they are proportional to the severity of COVID-19 (see, e.g., Aslan et al., Pneumonia volume 13, Article number: 14, 2021). The progression of the ARDS is generally shown in three overlapping stages: exudative stage, proliferative stage, and fibrotic stage. Exudative stage is represented by various inflammatory symptoms, including cytokine releases and influx of immune cells (e.g., neutrophils). Proliferative stage is characterized by early fibrotic changes, which often progresses to fibrotic stage. Fibrotic stage, which is an advanced stage of the ARDS, is characterized by intra-alveolar and interstitial fibrosis, increased collagen deposition, a prolonged period of ventilation-perfusion mismatching, and diminished compliance of the lung. (see, e.g., Walkey et al., Clin. Epidemiol., 2012; 4: 159-19). A significant portion of ARDS patients advances to proliferative stage and even to fibrotic stage, and the hallmarks of such stages, intra-alveolar and interstitial fibrosis, have been associated with a poor prognosis with high mortality and/or prolonged ventilator dependence.

Long non-coding transcripts (or long non-coding RNAs (lncRNAs)) are RNA segments that lack protein-coding capacity, yet mediate various regulatory mechanisms in cell cycle or cell metabolism by regulating transcription and/or post-transcriptional modification of various genes. As such dysregulation of certain long non-coding transcripts can be associated with an onset, development, or prognosis of a disease or a symptom of a disease. Alternatively and/or additionally, dysregulation of certain long non-coding transcripts can be a signature or indication of an onset, development, or prognosis of a disease or a symptom of a disease.

Some long non-coding transcripts affect development of certain types of fibrosis by promoting extracellular matrix (ECM) synthesis by affecting fibroblast cells in the tissue(s). In some aspects, as provided herein, certain long non-coding transcript is associated with onset, development, and/or prognosis of ARDS. In some aspects, certain long non-coding transcript is associated with onset, development, and/or prognosis of pulmonary fibrosis associated with ARDS. In some instances, the pulmonary fibrosis is induced by ARDS. In some instances, the pulmonary fibrosis is resulted from one or more symptoms or pathophysiology of ARDS.

In some aspects, the expression of the long noncoding transcripts disclosed herein is elevated in a subject affected by fibrosis (e.g., pulmonary fibrosis) associated with ARDS. In some aspects, the expression of the long noncoding transcripts disclosed herein is elevated in a subject affected by ARDS. In some aspects, the expression of the long noncoding transcripts disclosed herein is elevated in a subject affected by ARDS related to or resulted from COVID-19 infection. In some aspects, the expression of the long noncoding transcripts disclosed herein is elevated in a subject affected by COVID-19 infection. In some aspects, the expression of the long noncoding transcripts disclosed herein is elevated in a subject having an inflammation or increased immune response associated with onset or development of ARDS. In some aspects, the inflammation or increased immune response is represented or shown by increased infiltration of immune cells to the tissue (e.g., lung tissue), activation of immune cells in the lung tissue or to the lung tissue (including lung fibroblast), increased secretion or accumulation of inflammatory cytokines or chemokines in the lung tissue. In some specific aspects, the elevation is at least by 1%, 2%, 3%, 4%, 5%, 10%, 20%, 30%, 40%, 50%, 60%, 70%, 80%, 90%, 100%, 200%, 300%, 400%, 500%, 600%, 700%, 800%, 900%, or 1000%. In some aspects, the activity of the long noncoding transcripts disclosed herein is elevated in a subject affected by fibrosis associated with ARDS. In some specific aspects, the elevation is at least by 1%, 2%, 3%, 4%, 5%, 10%, 20%, 30%, 40%, 50%, 60%, 70%, 80%, 90%, 100%, 200%, 300%, 400%, 500%, 600%, 700%, 800%, 900%, or 1000%.

In some aspects, the elevated expression and/or activity of the long-noncoding transcript disclosed herein is associated with severity of an ARDS symptom in the subject. In some specific aspects, the elevated expression is associated with severity of labored and rapid breathing. In some aspects, the elevated expression is associated with severity of shortness of breath. In some aspects, the elevated expression is associated with severity of low blood pressure. In some aspects, the elevated expression is associated with severity of extreme tiredness.

In some aspects, the elevated expression and/or activity of the long-noncoding transcript disclosed herein is detected in the pulmonary myofibroblasts compared to fibroblasts. In some aspects, the elevated expression and/or activity of the long-noncoding transcript disclosed herein is detected in the induced pulmonary myofibroblasts compared to fibroblasts. In some instances, the expression level of the long-noncoding transcript is increased in the pulmonary myofibroblasts or induced pulmonary myofibroblasts at least 10%, 20%, 30%, 40%, 50%, 60%, 70%, 80%, 90%, 100%, 200%, 300%, 400%, 500% compared to the fibroblasts.

In some aspects, the long-noncoding transcript disclosed herein is associated with or modulates expression or activity of one or more fibrosis marker genes. In some instances, the fibrosis-related markers comprise smooth muscle a actin (ACTA2), alpha 1 chain of collagen type I (COL1A1), alpha 1 chain of collagen type 3 (COL3A1), fibroblast activation protein (FAP), fibronectin 1 (FN1), periostin (POSTN), or a combination thereof. In some instances, the increased expression of noncoding transcript disclosed herein is proportional to the increased expression of one or more fibrosis marker genes.

As such, in some aspects, inhibition of expression or activity of the long-noncoding transcript modulates expression of one or more fibrosis marker genes (e.g., ACTA2, COL1A1, COL3A1, FAP, FN1, POSTN, etc.). In some instances, inhibition of expression or activity of the long-noncoding transcript decreases expression of one or more fibrosis marker genes (e.g., ACTA2, COL1A1, COL3A1, FAP, FN1, POSTN, etc.). In some instances, inhibition of expression or activity of the long-noncoding transcript decreases expression of one or more fibrosis marker genes (e.g., ACTA2, COL1A1, COL3A1, FAP, FN1, POSTN, etc.) at least 5%, 10%, 15%, 20%, 30%, 40%, 50%, 60%, 70%, 80%, or 90% or more. In some instances, inhibition of expression or activity of the long-noncoding transcript decreases expression of one or more fibrosis marker genes (e.g., ACTA2, COL1A1, COL3A1, FAP, FN1, POSTN, etc.) at least 5%, 1, 1⁵%, 20%, 30%, 40%, 50%, 60%, 70%, 80%, or 90% or more from the increased expression level of the one or more fibrosis marker genes in the myofibroblast, induced myofibroblast, a cell affected by pulmonary fibrosis associated with ARDS, a cell affected by ARDS, a cell affected by COVID-19 related ARDS, a tissue affected by pulmonary fibrosis associated with ARDS, a tissue affected by ARDS, or a tissue affected by COVID-19 related ARDS. In some instances, inhibition of expression or activity of the long-noncoding transcript modulates a fibrosis status of a tissue affected by pulmonary fibrosis associated with ARDS, a tissue affected by ARDS, or a tissue affected by COVID-19 related ARDS. In some instances, inhibition of expression or activity of the long-noncoding transcript reduces or prevents progress of a fibrosis status of a tissue affected by pulmonary fibrosis associated with ARDS, a tissue affected by ARDS, or a tissue affected by COVID-19 related ARDS. In some instances, inhibition of expression or activity of the long-noncoding transcript reverses progress of a fibrosis status of a tissue affected by pulmonary fibrosis associated with ARDS, a tissue affected by ARDS, or a tissue affected by COVID-19 related ARDS.

In some aspects, the long noncoding transcript disclosed herein is transcribed from a genomic region located at the same chromosome with one or more fibrosis marker genes. In some aspects, the long-noncoding transcript disclosed herein is transcribed from a genomic region where one or more single nucleotide polymorphism (SNP) associated with COVID-19 risk variants are located. In some aspects, the long-noncoding transcript disclosed herein is transcribed from a genomic allele where one or more single nucleotide polymorphism (SNP) associated with COVID-19 risk variants are located. As used herein, COVID-19 risk variant is any genetic or epigenetic variation, mutation, or modification that contributes to COVID-19 susceptibility, severity, and/or mortality. In some embodiments, a certain aspect of COVID-19 susceptibility, severity, and/or mortality relates to respiratory failure. In some embodiments, a certain aspect of COVID-19 susceptibility, severity, and/or mortality relates to an increased risk of respiratory failure. In some embodiments, respiratory failure is defined as a condition of a subject in which a medical treatment comprising the use of oxygen supplementation or mechanical ventilation is administered to the subject as an indicated medical intervention. In some embodiments, a severity of respiratory failure is graded according to a maximum level of respiratory support received by a subject at any point during hospitalization (e.g., in order of increasing severity of respiratory support: supplemental oxygen therapy only, noninvasive ventilatory support, invasive ventilatory support, extracorporeal membrane oxygenation). In some embodiments, a certain aspect of COVID-19 susceptibility, severity, and/or mortality relates to an increased risk of respiratory failure of at least two-fold. In some embodiments, a certain aspect of COVID-19 susceptibility, severity, and/or mortality relates to interstitial pneumonia. In some embodiments, a certain aspect of COVID-19 susceptibility, severity, and/or mortality relates to bilateral interstitial pneumonia. n some embodiments, a certain aspect of COVID-19 susceptibility, severity, and/or mortality relates to an elevated risk of bilateral interstitial pneumonia. In some instances, the COVID-19 risk variant is identified from genome-wide association study (GWAS).

In some aspects, the genomic region in which the long-noncoding transcript (lncRNAs) are transcribed as disclosed herein is located within chromosome 3. In some aspects, the genomic region in which the long-noncoding transcript (lncRNAs) are transcribed as disclosed herein is annotated according to the Human Genome Resources at NCBI. In some embodiments, the genomic region in which the long-noncoding transcript (lncRNAs) are transcribed as disclosed herein is annotated according to the Human Genome Resources at NCBI according to Assembly: GRCh30.p14 (GCF_000001405.40). In some aspects, the genomic region in which the long-noncoding transcript (lncRNAs) are transcribed as disclosed herein is located within chr3:45,806,503 to chr3:45,834,110. In some aspects, the genomic region in which the long-noncoding transcript (lncRNAs) are transcribed as disclosed herein is located within chr3:45,806,503 to chr3:45,831,916. In some aspects, the genomic region in which the long-noncoding transcript (lncRNAs) are transcribed as disclosed herein is located within chr3:45,796,552 to chr3: 45,834,110. In some aspects, the genomic region in which the long-noncoding transcript (lncRNAs) are transcribed as disclosed herein is located within chr3:45,796,552 to chr3: 45,831,916. In some aspects, the genomic region in which the long-noncoding transcript (lncRNAs) are transcribed as disclosed herein is located within chr3:45,796,552 to chr3: 45,829,603. In certain aspects, the genomic region in which the long-noncoding transcript (lncRNAs) are transcribed as disclosed herein is located within LOC107986083 (chr3: 45,817,379-45,827,511). In some aspects, the long-noncoding transcript disclosed herein is transcribed using Crick Strand of the genomic region as a template.

In some aspects, the long noncoding transcript disclosed herein comprises a nucleic acid sequence of at least a portion of XR_001740681.1 (NCBI). In some aspects, the long noncoding transcript disclosed herein comprises a nucleic acid sequence of at least a portion of locus ENSG00000288720 (Gencode/ENSEMBL). In some aspects, the long noncoding transcript disclosed herein comprises a nucleic acid sequence of at least a portion of transcript ENST00000682011.1 (Gencode/ENSEMBL). In other aspects, the long noncoding transcript disclosed herein comprises a nucleic acid sequence of at least a portion of transcript ENST00000684202.1 (Gencode/ENSEMBL). In other aspects, the long noncoding transcript disclosed herein comprises a nucleic acid sequence of at least a portion of transcript RP11-852E15.3 (Gencode).

In some aspects, the lncRNA associated with ARDS induced pulmonary fibrosis is transcribed from the genomic region LOC107986083 (chr3: 45,817,379-45,827,511). In some aspects, the lncRNA associated with ARDS induced pulmonary fibrosis is transcribed from the Crick strand of the genomic region LOC107986083 (chr3: 45,817,379-45,827,511). In some aspects, the lncRNA associated with ARDS induced pulmonary fibrosis comprises at least a portion of XR_001740681.1 (NCBI). In some aspects, the lncRNA associated with ARDS induced pulmonary fibrosis comprises at least a portion of locus ENSG00000288720 (Gencode/ENSEMBL). In some aspects, the lncRNA associated with ARDS induced pulmonary fibrosis comprises at least a portion of transcript ENST00000682011.1 (Gencode/ENSEMBL). In other aspects, the lncRNA associated with ARDS induced pulmonary fibrosis comprises at least a portion of transcript ENST00000684202.1 (Gencode/ENSEMBL). In other aspects, the lncRNA associated with ARDS induced pulmonary fibrosis comprises at least a portion of transcript RP11-852E15.3 (Gencode). In some aspects, the lncRNA associated with ARDS induced pulmonary fibrosis is also associated with leukocyte infiltration accompanying ARDS. In some aspects, the lncRNA associated with ARDS induced pulmonary fibrosis is also associated with leukocyte infiltration preceding onset of ARDS.

In some aspects, the lncRNA associated with ARDS induced pulmonary fibrosis is transcribed from the human genomic region on chromosome 3 (chr3: 45,796,552-chr3: 45,834,110). In some aspects, the lncRNA associated with ARDS induced pulmonary fibrosis is transcribed from the human genomic region on chromosome 3 (chr3: 45,817,792-chr3: 45,834,110). In some aspects, the lncRNA associated with ARDS induced pulmonary fibrosis is transcribed from the genomic region LOC126806670 chr3:45,817,792-chr3: 45,818,991). In some aspects, In some aspects, the lncRNA associated with ARDS induced pulmonary fibrosis is transcribed from the Crick strand of the human genomic region on chromosome 3. In some embodiments, the lncRNA associated with ARDS induced pulmonary fibrosis is transcribed from the human genomic region on chromosome 3 in which the genome location is annotated according to GRCh37/hgl9. In some aspects, the lncRNA associated with ARDS induced pulmonary fibrosis comprises at least a portion of an enhancer contained within LOC126806670.

In some aspects, the lncRNA associated with ARDS induced pulmonary fibrosis is also associated with leukocyte infiltration accompanying ARDS. In some aspects, the lncRNA associated with ARDS induced pulmonary fibrosis is also associated with leukocyte infiltration preceding onset of ARDS.

In some aspects, the long noncoding transcript disclosed herein comprises a single nucleotide polymorphism (SNP) associated with the ARDS. In some aspects, the long noncoding transcript disclosed herein comprises a single nucleotide polymorphism (SNP) associated with the COVID-19 risk. In some aspects, the long noncoding transcript disclosed herein comprises a single nucleotide polymorphism (SNP) that is an COVID-19 risk variant SNP. In some aspects, the long noncoding transcript disclosed herein is transcribed from a genomic region in which one or more single nucleotide polymorphism (SNP) associated with the COVID-19 risk is located. In some aspects, the long noncoding transcript disclosed herein is transcribed from a genomic region in which one or more single nucleotide polymorphism (SNP) that is an COVID-19 risk variant SNP. In some embodiments, the COVID-19 risk variant SNP has been identified in a GWAS study. In some embodiments, the COVID-19 risk variant SNP is rs1040770, rs1886814, rs72711165, rs10774671, rs77534576, rs1819040, rs74956615, rs2109069, rs13050728, or rs17713054. In some embodiments, the COVID-19 risk variant SNP is rs17713054. In some embodiments, the COVID-19 risk variant SNP is described in (Severe Covid-19 GWAS Group et al. Genomewide Association Study of Severe Covid-19 with Respiratory Failure. N Engl J Med. 2020 Oct. 15; 383(16):1522-1534) which is herein incorporated by reference for techniques of identifying and characterizing COVID-19 risk variant SNPs. In some embodiments, the COVID-19 risk variant SNP is described in (Downes D J et al. Identification of LZTFL1 as a candidate effector gene at a COVID-19 risk locus. Nat Genet. 2021 November; 53(11):1606-1615) which is herein incorporated by reference for techniques of identifying and characterizing COVID-19 risk variant SNPs. In some embodiments, the COVID-19 risk variant SNP is linked to a causative allele affecting a component of COVID-19 risk in a subject. In some embodiments, the COVID-19 risk variant SNP allele is in linkage disequilibrium to a nearby allele of a genetic variant affecting a component of COVID-19 risk in a subject. In some embodiments, a long noncoding transcript (lncRNA) comprises a COVID-19 risk variant SNP. In some embodiments, a long noncoding transcript (lncRNA) does not comprise a COVID-19 risk variant SNP. In some embodiments, a COVID-19 risk variant SNP is in a genomic region in close proximity to a long noncoding transcript (lncRNA)affecting a component of COVID-19 risk in a subject. In some embodiments, COVID-19 risk includes risk of a subject exhibiting one or more symptoms of COVID-19. In some embodiments, COVID-19 risk includes risk of a subject exhibiting a certain degree of severity of one or more symptoms of COVID-19. In some aspects, the long noncoding transcript disclosed herein comprises rs17713054. In some aspects, the long noncoding transcript disclosed herein does not comprise rs17713054. In some aspects, increased CEBPβ binding at an enhancer of the long non-coding transcript disclosed herein due to rs1773054 risk variant facilitates PU.1 binding and transactivation of enhancer and the expression of the long noncoding transcript disclosed herein.

In some instances, the long noncoding transcript is preferentially or highly expressed in pulmonary tissues in a healthy individual. In some instances, the expression level of the long noncoding transcript is at least 10%, 20%, 30%, 40%, 50% higher in pulmonary tissues than other tissues in a healthy individual. In some instances, the long noncoding transcript is ubiquitously expressed in various body tissues in a healthy individual. In some instances, the expression of the long noncoding transcript is preferentially or specifically increased in the pulmonary tissues than other tissues in an individual affected by pulmonary fibrosis associated with or induced by ARDS associated with COVID-19 infection. In some instances, the expression level of long noncoding transcript is at least 10%, 20%, 30%, 40%, 50% higher in the pulmonary tissues than other tissues in an individual affected by pulmonary fibrosis associated with or induced by ARDS associated with COVID-19 infection. In some instances, the expression of the long noncoding transcript is preferentially or specifically increased in the pulmonary tissues than other tissues in an individual affected by COVID-19 infection. In some instances, the expression level of long noncoding transcript is at least 10%, 20%, 30%, 40%, 50% higher in the pulmonary tissues than other tissues in an individual affected by COVID-19 infection. In some embodiments, the expression level of long noncoding transcript in the pulmonary tissue is in pulmonary mesenchymal cells. In some embodiments, the expression level of long noncoding transcript in the pulmonary tissue is in pulmonary fibroblasts. In some embodiments, the pulmonary fibroblasts are interstitial resident fibroblasts (iReFs). In some embodiments, the expression level of long noncoding transcript in the pulmonary tissue is in myofibroblasts. In some embodiments, the expression level of long noncoding transcript in the pulmonary tissue is in matrix fibroblasts. In some embodiments, the expression level of long noncoding transcript in the pulmonary tissue is in lipofibroblasts. In some embodiments, the expression level of long noncoding transcript in the pulmonary tissue is in fibrocytes. In some embodiments, the expression level of long noncoding transcript in the pulmonary tissue is in alveolar niche cells. In some embodiments, the expression level of long noncoding transcript in the pulmonary tissue is in alveolar niche progenitor cells. In some embodiments, the expression level of long noncoding transcript in the pulmonary tissue is in respiratory epithelial cells. In some embodiments, the expression level of long noncoding transcript in the pulmonary tissue is in ciliated pseudostratified columnar epithelium. In some embodiments, the cells types in ciliated pseudostratified columnar epithelium in which the long noncoding transcript is expressed are ciliated cells, goblet cells, basal cells, brush cells, or neuroendocrine cells, or any combination thereof. In some embodiments, the expression level of long noncoding transcript in the pulmonary tissue is in Type I pneumocytes (alveolar type I epithelial cells), Type II pneumocytes (alveolar type II epithelial cells), both Type I pneumocytes and Type II pneumocytes. In some embodiments, the expression level of long noncoding transcript in the pulmonary tissue is in alveolar macrophages, neutrophils, T cells, or any combination thereof. In some embodiments, the expression level of long noncoding transcript in the pulmonary tissue is in endothelial cells.

### Modulation of Long Noncoding Transcript

In some aspects, the modulator disclosed herein modifies the genomic DNA that is transcribed to the long noncoding transcript disclosed herein. In some instances, the modulator disclosed herein modifies a portion of such genomic DNA so that the genomic DNA is mutated.

In some instances, the modulator disclosed herein modifies a portion of such genomic DNA so that the transcription level is suppressed. Accordingly, in some instances, the modulator reduces the amount of the long noncoding transcript. In some instances, the modulator disclosed herein modifies a portion of such genomic DNA so that the transcription level is activated. In some aspects, the modulator disclosed herein modifies the long noncoding transcript disclosed herein.

In some instances, the modulator disclosed herein modifies a portion of such long noncoding transcript so that the long noncoding transcript is degraded. In some instances, the modulator disclosed herein modifies a portion of such long noncoding transcript so that the long noncoding transcript is retained longer. In some instances, the modulator disclosed herein modifies a portion of such long noncoding transcript so that the activity of the long noncoding transcript on downstream reactions or pathways is suppressed. Accordingly, in some instances, the modulator reduces the activity of the long noncoding transcript. In some instances, the modulator disclosed herein modifies a portion of such long noncoding transcript so that the activity of the long noncoding transcript on downstream reactions or pathways is activated.

In some aspects, a modulator of the long noncoding transcript increases or decreases of the expression or activity of the long noncoding transcript. In some instances, a modulator of the long noncoding transcript prevent increases or decreases of the expression or activity of the long noncoding transcript. In some instances, a modulator of the long noncoding transcript reverses the pathological changes of the expression or activity of the long noncoding transcript. In some instances, a modulator of the long noncoding transcript reverses the pathological changes of the expression or activity of the long noncoding transcript at least ±5%, ±10%, ±15%, ±20%, ±25% of the normal expression or activity of the long noncoding transcript before the onset or development of the pathological symptoms or diseases (e.g., baseline level), or at least ±5%, ±10%, ±15%, ±20%, ±25% of the expression or activity of the long noncoding transcript of a healthy individual or healthy tissue (e.g., baseline level). In some aspects, the modulator disclosed herein reduces the elevated amount of long noncoding transcript by at least 30%, at least 40%, at least 50%, at least 60%, at least 70%, at least 80%, at least 90% in a cell or a tissue of the subject, wherein the elevated amount is associated with ARDS. In some aspects, the modulator disclosed herein reduces the elevated activity of long noncoding transcript by at least 30%, at least 40%, at least 50%, at least 60%, at least 70%, at least 80%, at least 90% in a cell or a tissue of the subject, wherein the elevated amount is associated with ARDS.

In some aspects, a long noncoding transcript modulates transcription of one or more downstream gene, which expression is a marker for a disease onset, development, or prognosis.

As such, in some instances, a modulator of the long noncoding transcript affects (e.g., increases or decreases) the expression or activity of the downstream gene or the long noncoding transcript.

In some instances, a modulator of the long noncoding transcript increases the expression or activity of one or more downstream genes at least 10%, 20%, 30%, 40%, 50%, 60%, 70%, 80%, 90%, 100%, or higher. In some instances, a modulator of the long noncoding transcript decreases the expression or activity of one or more downstream genes at least 10%, 20%, 30%, 40%, 50%, 60%, 70%, 80%, 90%, 100%, or higher. In some instances, a modulator of the long noncoding transcript reverses the pathological changes of the expression or activity of one or more downstream genes. In some instances, a modulator of the long noncoding transcript reverses the pathological changes of the expression or activity of one or more downstream genes to the level differing no more than ±5%, ±10%, ±15%, ±20%, ±25% from the normal expression or activity of the long noncoding transcript before the onset or development of the pathological symptoms or diseases. In some instances, a modulator of the long noncoding transcript reverses the pathological changes of the expression or activity of one or more downstream genes to the level differing no more than at least +5%, ±10%, ±15%, ±20%, ±25% from the normal expression or activity of the long noncoding transcript of a healthy individual or healthy tissue. In some embodiments, the downstream gene is a marker gene for pulmonary fibrosis associated with or induced by ARDS, e.g., ARDS associated with COVID-19 infection, e.g., ACTA2, COL1A1, COL3A1, FAP, FN1, POSTN, etc. In some embodiments, the downstream gene is a marker gene for pulmonary immune cell infiltration associated with or induced by ARDS, e.g., ARDS associated with COVID-19 infection. In some embodiments, the downstream gene is a marker gene associated with leukocyte migration. In some embodiments, the downstream gene is a marker gene associated with leukocyte chemotaxis. In some embodiments, the downstream gene is a marker gene associated with leukocyte proliferation. In some embodiments, the downstream gene is a marker gene associated with leukocyte activation. In some embodiments, the one or more downstream genes are included as part of an IPF gene signature. In some embodiments, the IPF gene signature is a human IPF gene signature constructed from expression data derived from human cells. In some embodiments, the IPF gene signature is a mouse IPF gene signature constructed from expression data derived from mouse cells. In some embodiments, the one or more downstream genes included as part of an IPF gene signature are upregulated in pulmonary fibrosis associated with or induced by ARDS, or in ARDS associated with COVID-19 infection. In some embodiments, the one or more upregulated downstream genes included as part of a human IPF gene signature are listed in Table 1. In some embodiments, the one or more downstream genes included as part of a human or mouse IPF gene signature are downregulated in pulmonary fibrosis associated with or induced by ARDS, or in ARDS associated with COVID-19 infection. In some embodiments, the one or more downregulated downstream genes included as part of a human IPF gene signature are listed in Table 2. In some embodiments, the one or more downstream genes included as part of an IPF gene signature comprise one or more upregulated downstream genes and one or more downregulated downstream genes. In some instances, a modulator of the long noncoding transcript decreases the expression or activity of one or more downstream genes listed in Table 1. In some instances, a modulator of the long noncoding transcript increases the expression or activity of one or more downstream genes listed in Table 2. In some instances, a modulator of the long noncoding transcript decreases the expression or activity of one or more downstream genes listed in Table 1 and increases the expression or activity of one or more downstream genes listed in Table 2. In some embodiments, the IPF gene signature is constructed according to Example 7 herein.

TABLE 1
List of Upregulated genes in the human IPF gene signature

| | | | | | | |
|---|---|---|---|---|---|---|
| ABCB4 | CH507-9B2.1 | FAM133A | ITGA9 | MRC2 | PYCR1 | SNRPN |
| ACOX2 | CHD3 | FAM13C | ITGAV | MROH8 | QPCT | SORCS2 |
| ACTA2 | CHN1 | FAM171B | ITGB3 | MSC | RAB30 | SOX4 |
| ACTG2 | CHPF | FAM198B- | ITGB5 | MSC-AS1 | RABL2A | SPARC |
| | | AS1 | | | | |
| ACTN1 | CHRDL2 | FAM227A | ITGBL1 | MT1F | RAMP1 | SPECC1 |
| ACVR1 | CHST6 | FAM227B | ITM2C | MTHFD1L | RAP2B | SPON1 |
| ADAM12 | CHSY1 | FAM229B | JAK3 | MXRA8 | RASA4 | SPSB1 |
| ADAM19 | CILP | FAM66D | JAZF1 | MYH11 | RASA4B | SRGAP3 |
| ADAMTS10 | CILP2 | FAM98A | KCNA2 | MYOCD | RASD2 | SRRM3 |
| ADAMTS16 | CKAP4 | FANK1 | KCND1 | MYOM1 | RASGRP3 | SSC5D |
| ADAMTS6 | CLCA2 | FAP | KCND3 | MYOSLID | RASL11B | SSPN |
| ADCY5 | CLIP3 | FBLIM1 | KCNH1 | NAALADL2 | RBP1 | SSR4 |
| ADGRA2 | CLMP | FBLN1 | KCNN4 | NAP1L3 | RCAN2 | ST8SIA2 |
| ADORA1 | CLSTN2 | FBLN2 | KCTD11 | NBAT1 | RCC2 | STEAP3-AS1 |
| ADSS1 | CLSTN3 | FBN1 | KDELR2 | NBEA | RCN3 | STK38L |
| AEBP1 | CLU | FBXL13 | KDM5B | NDUFA6-DT | RCOR2 | SUFU |
| AGT | CNPY4 | FBXL22 | KIAA1755 | NEK11 | RERG | SUGCT |
| AKNA | COL10A1 | FBXO32 | KIF26B | NFATC4 | RGS2 | SULF2 |
| ALDH18A1 | COL14A1 | FGF14 | KIF3C | NIPSNAP3B | RIMS2 | SYNDIG1 |
| ALDH1L2 | COL15A1 | FGF18 | KIF5A | NLGN2 | RN7SL689P | SYT12 |
| ALKAL1 | COL16A1 | FHL2 | KIF9-AS1 | NNMT | RNF150 | TACR1 |
| ALPK2 | COL1A1 | FILIP1L | KLHL4 | NOTCH3 | ROBO1 | TAGLN |
| ANGPTL2 | COL1A2 | FKBP10 | KSR1 | NPAS2 | ROBO2 | TENM3 |
| ANK2 | COL24A1 | FKBP11 | LCA5L | NR2F1-AS1 | ROR2 | TENM4 |
| ANKH | COL27A1 | FKBP14 | LDB3 | NRP2 | RP1- | TEX9 |
| | | | | | 152L7.5 | |
| ANKRD20A5P | COL3A1 | FKBP7 | LDLRAD4 | NYNRIN | RP1- | TF |
| | | | | | 228H13.5 | |
| ANO4 | COL4A2-AS2 | FLRT2 | LEF1 | OGN | RP1- | TGFB3 |
| | | | | | 37C10.7 | |
| ANTXR1 | COL5A1 | FMO3 | LGMN | OLFM2 | RP11- | TGFBI |
| | | | | | 1151B14.4 | |
| AOPEP | COL5A2 | FMOD | LGR4 | OLFML2B | RP11- | THBS2 |
| | | | | | 119F7.5 | |
| APLP1 | COL6A1 | FN1 | LHPP | OLFML3 | RP11- | THBS3 |
| | | | | | 125O18.1 | |
| ARF4 | COL6A2 | FNDC5 | LINC00475 | OMD | RP11- | TIMP1 |
| | | | | | 145A3.1 | |
| ARHGEF25 | COL6A3 | FRK | LINC00519 | OMG | RP11- | TMED3 |
| | | | | | 167N4.2 | |
| ARMCX2 | COL7A1 | FRMD5 | LINC00535 | OSBPL10 | RP11- | TMEM117 |
| | | | | | 212I21.3 | |
| ARMH4 | COL9A2 | FRMD6 | LINC00578 | OSR2 | RP11- | TMEM119 |
| | | | | | 229O3.1 | |
| ASIC1 | COLQ | FRZB | LINC00632 | P3H1 | RP11- | TMEM132A |
| | | | | | 329B9.4 | |
| ASPN | COMP | FSTL1 | LINC00643 | P3H3 | RP11- | TMEM182 |
| | | | | | 348N5.7 | |
| ASS1 | COPZ2 | FUT8 | LINC01013 | P3H4 | RP11- | TMEM190 |
| | | | | | 403A21.1 | |
| ASTN1 | CPE | FZD7 | LINC01133 | P4HA3 | RP11- | TMEM231 |
| | | | | | 498C9.13 | |
| ASTN2 | CPQ | GABRB3 | LINC01138 | PAK3 | RP11- | TMEM263 |
| | | | | | 54O7.1 | |
| ATAT1 | CPZ | GAL | LINC01503 | PAMR1 | RP11- | TMEM45A |
| | | | | | 54O7.16 | |
| ATG13 | CRACD | GALNT1 | LINC01614 | PAPPA | RP11- | TMEM59L |
| | | | | | 567M16.1 | |
| ATP1A2 | CREB3L2 | GALNT16 | LINC01615 | PAPPA-AS1 | RP11- | TMSB15B |
| | | | | | 624L4.1 | |
| ATP2A3 | CRLF1 | GAN | LINC01711 | PAPPA2 | RP11- | TNC |
| | | | | | 867G23.10 | |
| AZIN2 | CROT | GAP43 | LINC01943 | PARD6G | RP11- | TNFRSF21 |
| | | | | | 893F2.5 | |
| B3GALNT2 | CSGALNACT1 | GAS6-AS1 | LINC02544 | PAX6 | RP11- | TNFSF18 |
| | | | | | 92C4.6 | |
| B4GALT1 | CSMD2 | GAS7 | LINC02593 | PBLD | RP11- | TNFSF4 |
| | | | | | 999E24.3 | |
| B4GALT4 | CST1 | GASK1B | LINC02606 | PBXIP1 | RP3- | TP53 |
| | | | | | 337O18.9 | |
| BACE2 | CTHRC1 | GDF6 | LINC02694 | PCAT6 | RP4- | TP53INP1 |
| | | | | | 565E6.1 | |
| BBOF1 | CTSO | GDNF | LINC02731 | PCDH19 | RP4- | TP53TG1 |
| | | | | | 622L5.7 | |
| BCHE | CUL7 | GFUS | LMO4 | PCDHB12 | RP5- | TPST1 |
| | | | | | 1054A22.4 | |
| BCL9 | CXXC5 | GLI1 | LMOD1 | PCDHB14 | RUNX1 | TRAF5 |
| BCO2 | DACH2 | GLI2 | LNCTAM34A | PCDHB2 | SALL4 | TRAV30 |
| BGN | DACT1 | GLIS3 | LOX | PCDHB7 | SAMD11 | TRIM32 |
| BICC1 | DACT3 | GLT8D1 | LOXL2 | PCOLCE | SCG2 | TRIM62 |
| BLNK | DACT3-AS1 | GLT8D2 | LOXL3 | PCSK1 | SCG5 | TRO |
| BMERB1 | DDIT4 | GOLGA2 | LRIG3 | PCYOX1L | SCPEP1 | TRPS1 |
| BMP4 | DELEC1 | GOLM1 | LRP1 | PDCD4 | SCRG1 | TRPV4 |
| BMPR1B | DENND2B | GPC2 | LRRC15 | PDGFRB | SCUBE3 | TSKU |
| BNC2 | DERL3 | GPR153 | LRRC17 | PDIA3 | SDC3 | TSPAN11 |
| BOC | DGKA | GPR155 | LRRC27 | PDIA4 | SEC23A | TSPAN2 |
| BRINP3 | DGKI | GPR173 | LRRC4C | PDLIM3 | SEC24D | TSPAN6 |
| C16orf71 | DMD | GPR183 | LTBP1 | PDLIM4 | SEC31A | TTC3 |
| C1QTNF3 | DMGDH | GPR78 | LTBP2 | PDLIM7 | SEL1L3 | TTC9 |
| C1QTNF6 | DNAH1 | GPRACR | LTBP3 | PDZRN3 | SELENOM | TTLL1 |
| C1R | DNAJB13 | GPX7 | LUM | PGBD5 | SEMA3C | TTLL11 |
| C1RL | DNAJB5 | GPX8 | LUZP2 | PGM2L1 | SEPTIN6 | TTYH3 |
| C1S | DNAJC12 | GRIA3 | MAGED1 | PGM5 | SERPINE2 | TUB |
| C1orf122 | DNAJC22 | GSEC | MAGED2 | PKP1 | SERPINF1 | TWIST1 |
| C1orf54 | DNM1 | GSN | MAGED4 | PLCB4 | SERPINI1 | TXLNB |
| C20orf85 | DNMT3A | GXYLT2 | MAGED4B | PLEKHA6 | SESN3 | UCHL1 |
| C4A | DPT | HAPLN3 | MAGEL2 | PLN | SFRP4 | UNC5C |
| C4B | DPYSL3 | HEPH | MAP3K4-AS1 | PLOD1 | SGCA | VASH2 |
| C5orf66 | DRC3 | HHAT | MAP3K7CL | PLOD2 | SGCD | VCAM1 |
| C7 | DRP2 | HIC1 | MAP6 | PLP1 | SGPL1 | VCAN |
| CABCOCO1 | DTX1 | HILPDA | MARCKSL1 | PLPP4 | SH3BGR | VCAN-AS1 |
| CACNA1C | DUXAP8 | HMCN1 | MCAM | PLPP5 | SH3PXD2A | VMP1 |
| CALB2 | DUXAP9 | HOXA3 | MDFI | PLTP | SH3PXD2B | VWA1 |
| CALD1 | EAF2 | HS3ST3A1 | MDK | PMEPA1 | SH3RF3 | VWCE |
| CAMK1D | ECM1 | HSP90B1 | MEG3 | PNMA8A | SH3RF3- | WHRN |
| | | | | | AS1 | |
| CAP2 | ECM2 | HSPB7 | MEG8 | PNMA8B | SHISAL1 | WIPI1 |
| CAPS | EFCAB12 | HTR2B | MEGF8 | PODN | SLC16A1 | WNT11 |
| CASC15 | EFCAB6 | HTRA1 | MEIS3 | PODNL1 | SLC16A2 | XBP1 |
| CASTOR3 | EFHB | HTRA3 | MEOX1 | POGLUT2 | SLC18B1 | XXYLT1 |
| CC2D2B | EFHC1 | HYDIN2 | MEX3A | POSTN | SLC1A4 | XXyac- |
| | | | | | | YX65C7_A.2 |
| CCDC144NL- | EFHC2 | ICOSLG | MFAP2 | POU2F2 | SLC22A17 | ZFP69B |
| AS1 | | | | | | |
| CCDC170 | EFNA4 | IDH2 | MFAP4 | PPIB | SLC29A3 | ZFPM2 |
| CCDC180 | EFNB3 | IER5L | MGP | PPIC | SLC2A1 | ZKSCAN7 |
| CCDC40 | ELAPOR1 | IFITM10 | MINAR1 | PPIC-AS1 | SLC2A10 | ZMAT3 |
| CCDC74A | EMILIN1 | IFT27 | MIR100HG | PPP1R12B | SLC35F2 | ZNF154 |
| CCDC8 | ENAH | IFT43 | MIR503HG | PRDM1 | SLC38A4 | ZNF423 |
| CCDC80 | ENTPD1 | IGDCC4 | MIR99AHG | PRDM6 | SLC44A3- | ZNF436 |
| | | | | | AS1 | |
| CCND2 | ENTPD1-AS1 | IGF1 | MLLT11 | PRDX4 | SLC46A3 | ZNF469 |
| CD24 | ENTPD7 | IGFBP3 | MMP10 | PRG4 | SLC7A5 | ZNF521 |
| CDH11 | EPHB2 | IGFBP7 | MMP11 | PRKACB | SLIT3 | ZNF561- |
| | | | | | | AS1 |
| CDH2 | EPHB3 | IL11 | MMP13 | PRUNE2 | SLITRK6 | ZNF711 |
| CDH6 | ERGIC3 | IL17RD | MMP2 | PSD2 | SMIM10L2A | ZNF827 |
| CDHR1 | ETV6 | INSYN1 | MMP21 | PSD3 | SMIM43 | ZNF846 |
| CDKN1A | EXTL1 | INSYN2A | MMP3 | PTCHD4 | SMO | ZNF853 |
| CELSR2 | EYA2 | INTS6L | MORC4 | PTGFRN | SMOX | |
| CERCAM | FAIM2 | IQCK | MPP2 | PTGIS | SNAI2 | |
| CFAP69 | FAM118A | ISLR | MPZL1 | PTHLH | SNCAIP | |
| CFH | FAM131B | ITGA11 | MRAS | PTK7 | SNED1 | |

TABLE 2
List of Downregulated genes in the human IPF gene signature

| | | | | | | |
|---|---|---|---|---|---|---|
| AASS | CCBE1 | EMP2 | IL6R | NTHL1 | RAPGEF4 | SVIP |
| ABCA3 | CCDC68 | ENOSF1 | IMP3 | NTNG1 | RASIP1 | SYNPO2L |
| ABHD17C | CCDC85C | EPDR1 | IMPA1 | NUDCD1 | RGMB | SYTL4 |
| ABHD5 | CCK | ERBB3 | INF2 | NUDT15 | RHBDF1 | TACC2 |
| AC007952.5 | CCND3 | ERRFI1 | INMT | NUDT16L2P | RHOF | TAOK3 |
| AC009237.16 | CD274 | ESM1 | IPO5 | NUP58 | RIPOR1 | TBC1D4 |
| AC009237.17 | CD36 | EVA1A | IRAK3 | OCLN | RNH1 | TBRG4 |
| AC009238.7 | CD47 | FABP5 | ITGA3 | OGDH | ROBO4 | TBX2-AS1 |
| AC009238.8 | CD55 | FAH | ITPK1 | OSGIN1 | RP1- | TCF21 |
| | | | | | 267D11.6 | |
| AC073130.3 | CDAN1 | FAM110A | ITPR3 | OSTF1 | RP11- | TDRD7 |
| | | | | | 2N1.2 | |
| AC124789.1 | CDCA7L | FAM111A | ITPRID2 | P2RY1 | RP11- | TEAD4 |
| | | | | | 432J24.5 | |
| ACADS | CDKL1 | FAM160A1 | KAT2B | P3H2 | RP11- | TEC |
| | | | | | 61J19.5 | |
| ACAT1 | CDKL2 | FAM167A | KHDRBS3 | PAK4 | RP11- | TENT5B |
| | | | | | 63G10.4 | |
| ACER3 | CDKN2AIPNL | FAM234B | KIF17 | PALB2 | RP11- | TFPI |
| | | | | | 867O8.11 | |
| ACKR4 | CDKN2D | FASN | KIFC3 | PALM2AKAP2 | RP11- | TGFBR3 |
| | | | | | 93B14.10 | |
| ACP3 | CELF2 | FEM1C | KLF15 | PAQR5 | RP11- | TJP2 |
| | | | | | 96C23.10 | |
| ACVRL1 | CENPX | FGD4 | KLF6 | PARP12 | RP11- | TLR3 |
| | | | | | 96C23.5 | |
| ADCY8 | CEP72 | FH | KLF9 | PCID2 | RP3- | TM4SF4 |
| | | | | | 331H24.6 | |
| ADI1 | CEP85 | FKBP4 | KNSTRN | PCYT2 | RP3- | TMBIM1 |
| | | | | | 342P20.2 | |
| ADPRH | CERS2 | FLII | LAMA3 | PDE12 | RP3- | TMEM106C |
| | | | | | 403A15.5 | |
| ADRA1B | CFL2 | FLRT3 | LDLR | PDE4DIPP2 | RPS6KA1 | TMEM192 |
| ADRB2 | CGN | FLT1 | LETM2 | PDP2 | RPS6KA2 | TMEM200B |
| AFAP1L1 | CHAC2 | FLVCR2 | LIMD1 | PEAR1 | RTN4 | TMEM245 |
| AFDN | CHPT1 | FN3K | LINC00472 | PECR | RTTN | TMEM53 |
| AGPAT2 | CHRAC1 | FOLR3 | LINC00513 | PEG10 | S100A3 | TMEM62 |
| AGPAT3 | CIT | FZD5 | LINC01224 | PGAM5 | SASH1 | TMPO |
| AHNAK | CITED2 | GALE | LINC01273 | PGAP6 | SCML1 | TMTC1 |
| AKAP1 | CLDN12 | GALK1 | LINC02185 | PHLPP1 | SEMA3E | TNNT1 |
| ALCAM | CLDN4 | GALNT3 | LIPH | PI4K2B | SFTA1P | TOM1L1 |
| ALS2CL | CLEC14A | GATA2 | LPCAT1 | PIEZO1 | SGO1 | TOR4A |
| AMD1 | CLEC3B | GBE1 | LRRN4 | PITPNM2 | SH2D5 | TOX |
| AMOTL2 | CLPP | GCAT | LSM6 | PITRM1 | SH3RF1 | TOX2 |
| ANKRD29 | CNTROB | GCDH | LSS | PKDCC | SHANK2 | TPRN |
| ANKRD33B | CPEB2 | GCNT2 | MACIR | PKN1 | SHMT1 | TPST2 |
| ANXA3 | CPNE3 | GEMIN4 | MAOA | PKN3 | SHROOM1 | TRHDE- |
| | | | | | | AS1 |
| APOL3 | CPNE8 | GIMAP2 | MAP3K6 | PLAAT3 | SIGIRR | TRIM25 |
| ARAP3 | CS | GIT1 | MAP4K2 | PLEKHJ1 | SLC14A1 | TRIM58 |
| ARHGAP29 | CSF2 | GJC2 | MAPK13 | PLEKHM1 | SLC16A12 | TRNP1 |
| ARHGAP6 | CTC- | GPAT3 | MBP | PLIN2 | SLC25A24 | TSPAN4 |
| | 308K20.1 | | | | | |
| ARHGEF26 | CTD- | GPD1L | MCCC1 | PMM1 | SLC25A25 | TTC39A |
| | 2003C8.2 | | | | | |
| ARL6IP6 | CTNNAL1 | GPD2 | MCFD2 | PODXL | SLC25A4 | TTLL12 |
| ATP11A | CTSH | GPER1 | MFSD13A | POLA2 | SLC25A5 | TTN |
| B3GALNT1 | CYB5A | GPN3 | MFSD2A | POLE | SLC44A2 | TUBGCP3 |
| BAIAP2 | CYC1 | GPR160 | MGLL | POPDC3 | SLC51B | TXNRD1 |
| BCAR3 | CYSTM1 | GPRC5A | MGST1 | PPA1 | SLC66A1L | TXNRD2 |
| BCAT2 | DAGLB | GRK5 | MIDEAS | PPARG | SLCO4A1 | UNC13B |
| BCL2L1 | DARS2 | GSAP | MLPH | PPFIBP1 | SLITRK2 | USP1 |
| BLVRB | DCLRE1A | HACD1 | MLX | PPL | SMAGP | USP13 |
| BMP2 | DCXR | HAGH | MME | PPM1F | SMC4 | USP31 |
| BMPER | DDX28 | HAUS4 | MRPL14 | PPP1R15A | SMIM29 | USP53 |
| BNIP3 | DENND3 | HCG27 | MTMR12 | PPP2R5A | SMURF2 | USP54 |
| BRI3 | DERA | HIF3A | MYO1C | PREX1 | SPC24 | UTP18 |
| BTBD6 | DGKE | HIRIP3 | NADK | PRKAR2B | SPC25 | UTRN |
| C10orf95- | DIAPH3 | HK2 | NAGS | PRKCE | SPRY2 | VEPH1 |
| AS1 | | | | | | |
| C13orf46 | DLL4 | HLA-E | NBEAL2 | PRKCQ-AS1 | SPRY4 | VSIG10 |
| C1GALT1 | DNHD1 | HMSD | NCAPH2 | PRKG2 | SPRY4- | VSIR |
| | | | | | AS1 | |
| C1orf115 | DNPEP | HNRNPF | NCKAP5 | PRLR | SPTBN1 | WDR5 |
| C1orf21 | DOCK5 | HOPX | NCKAP5- | PROSER2 | SPTLC3 | WNT3 |
| | | | AS2 | | | |
| C20orf27 | DOCK9 | HPCAL1 | NEDD4 | PRRG4 | SQOR | WWC1 |
| C4orf46 | DOT1L | HSBP1L1 | NEDD4L | PTPRQ | SSTR1 | WWC3 |
| CAMK2D | DSCAM | HSDL2 | NEMP1 | PVR | STAC | ZBTB42 |
| CAPN2 | DSEL | HYAL2 | NFKBIA | PYCARD | STARD3NL | ZC3H12C |
| CARD10 | DUSP6 | IER2 | NHSL1 | QDPR | STARD7 | ZDHHC12 |
| CASP4LP | E2F1 | IFIT3 | NIBAN2 | RAB11FIP1 | STARD8 | ZDHHC14 |
| CASZ1 | EBPL | IFIT5 | NIPA1 | RAB17 | STBD1 | ZDHHC7 |
| CAV1 | ECHDC3 | IGF2BP2 | NOTCH1 | RAB20 | STRADB | ZNF185 |
| CAVIN2 | EEF1AKNMT | IL15RA | NPC1 | RAB32 | STX11 | ZNF726P1 |
| CBR1 | EFL1 | IL17RE | NR3C2 | RAB3D | STX3 | ZNF792 |
| CC2D1B | EME2 | IL18 | NRGN | RAP1GAP2 | SUN2 | ZNF823 |

In some aspects, a long noncoding transcript modulates or expression of a long noncoding transcript is associated with cell phenotypes. As such, in some instances, a modulator of the long noncoding transcript affects cell phenotypes. For example, a modulator of the long noncoding transcript inhibits or prevents the morphological or physiological changes of a fibroblast to protomyofibroblast or to myofibroblast in a tissue affected by pulmonary fibrosis associated with or ARDS, e.g., ARDS associated with COVID-19 infection, or in a tissue affected by COVID-19 infection. In some instances, a modulator of the long noncoding transcript delays the morphological or physiological changes of a fibroblast to protomyofibroblast or to myofibroblast in a tissue affected by pulmonary fibrosis associated with or induced by ARDS, e.g., ARDS associated with COVID-19 infection, or in a tissue affected by COVID-19 infection. In some instances, a modulator of the long noncoding transcript reverses the morphological or physiological changes from protomyofibroblast or myofibroblast to fibroblast, or from fibroblast to protomyofibroblast in a tissue affected by pulmonary fibrosis associated with or induced by ARDS, e.g., ARDS associated with COVID-19 infection, or in a tissue affected by COVID-19 infection.

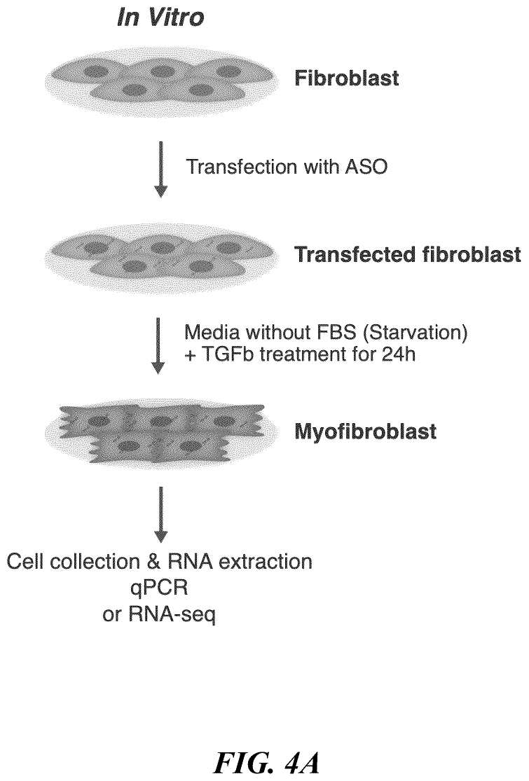

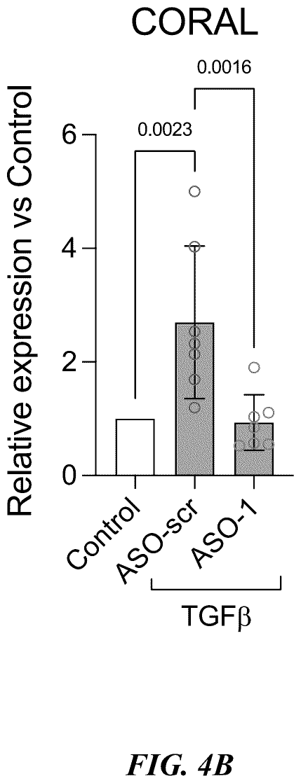

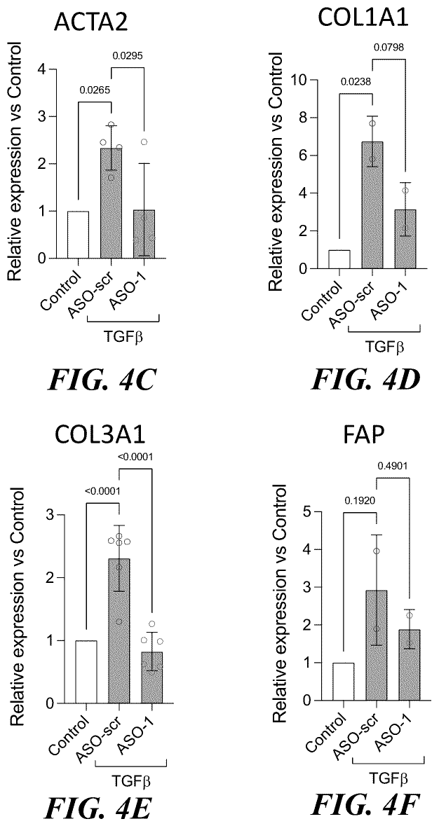

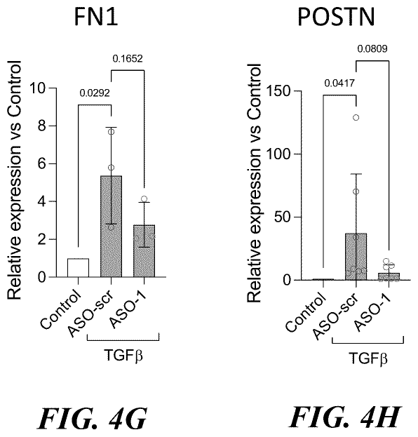

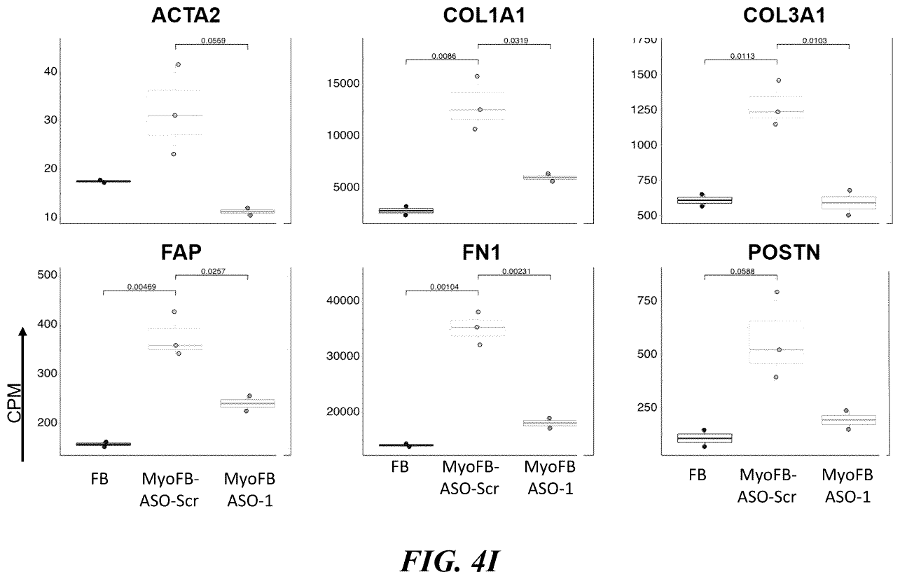

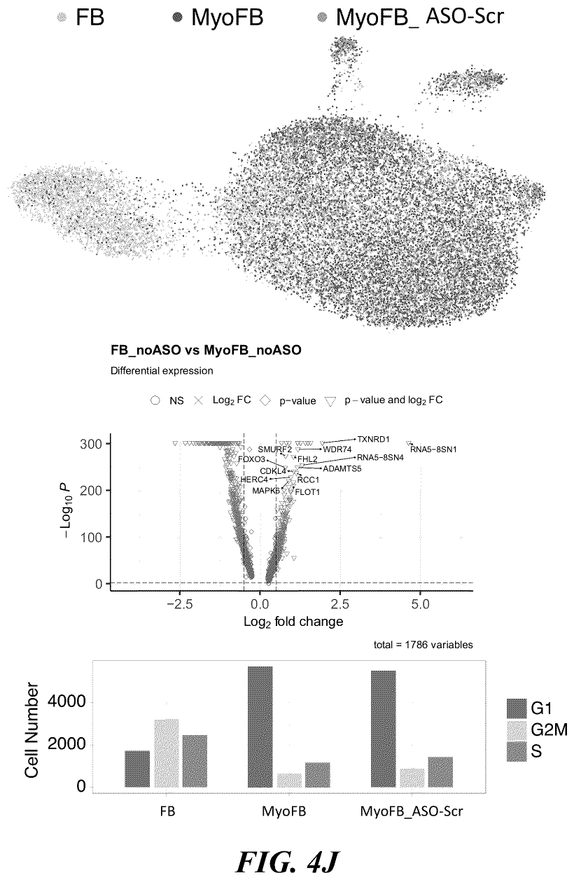

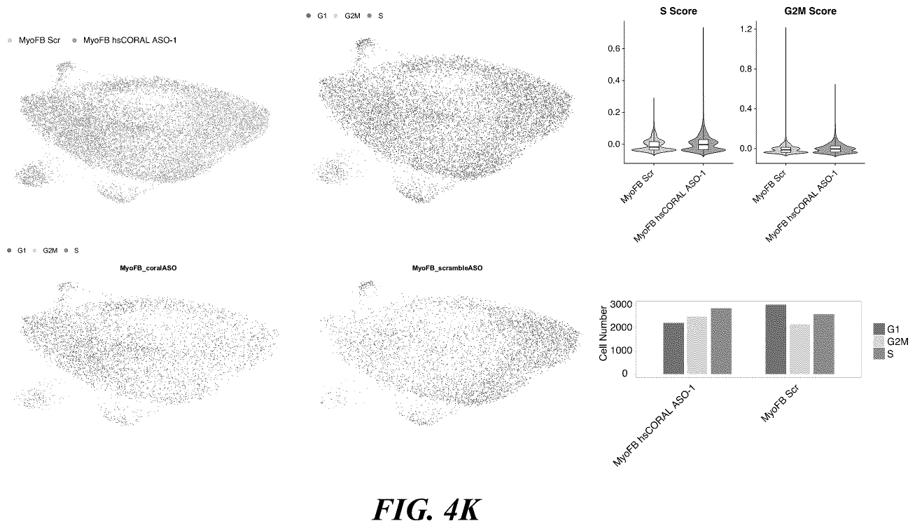

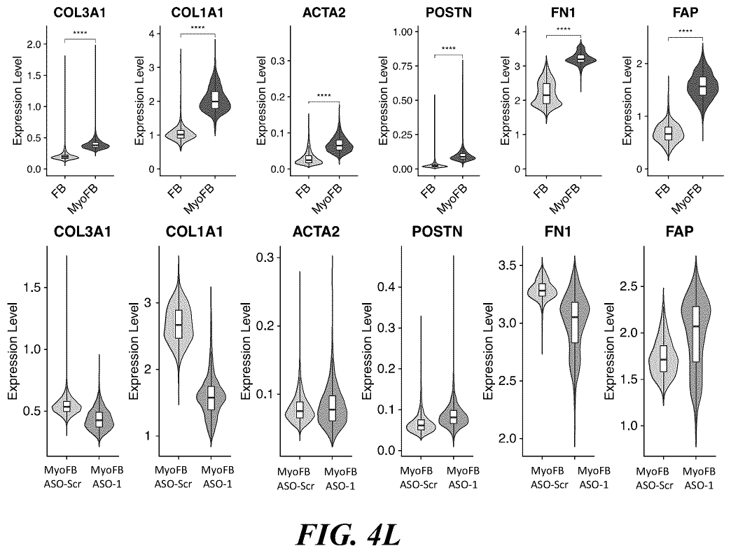

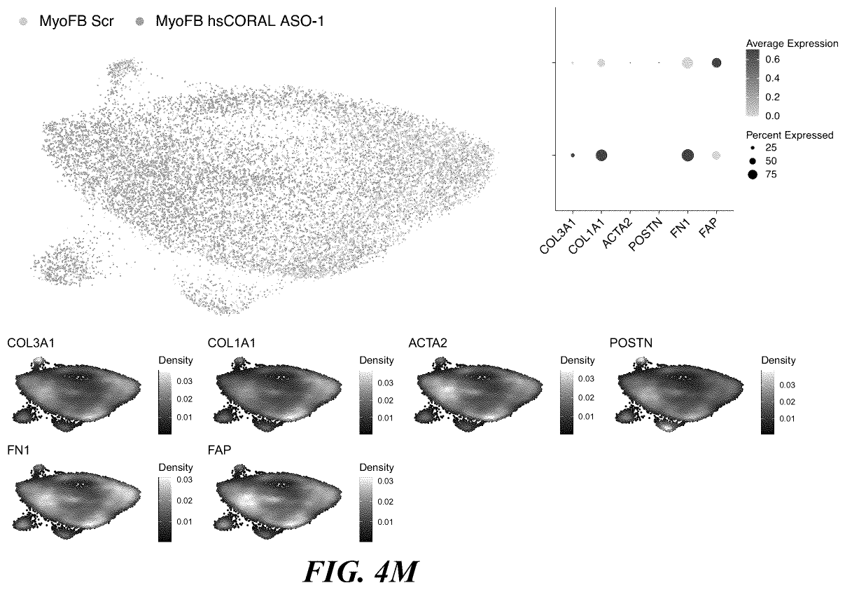

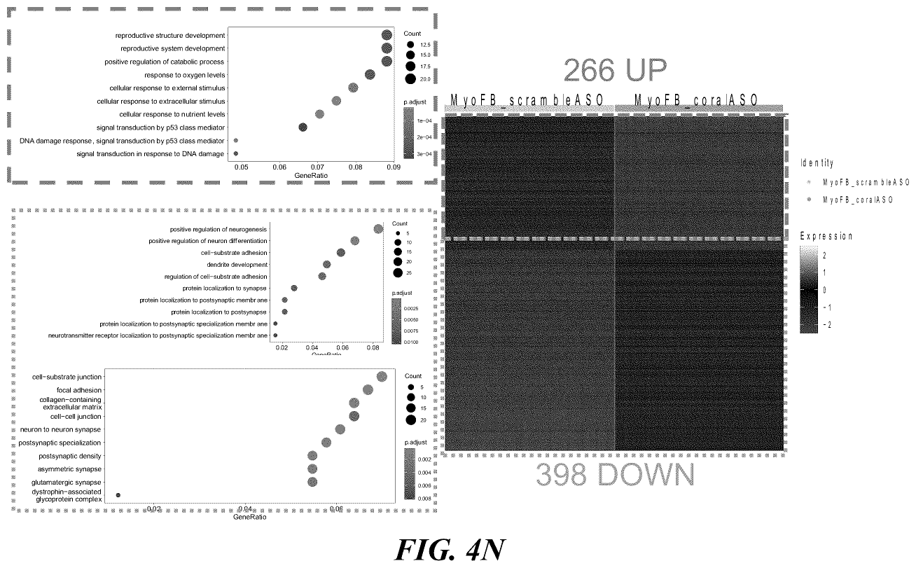

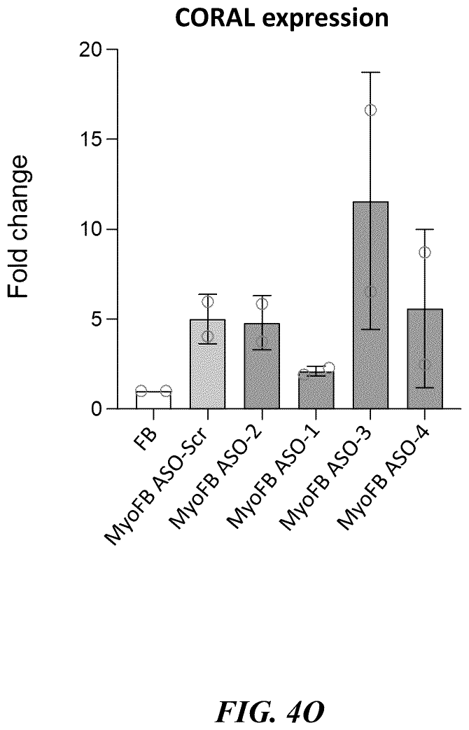

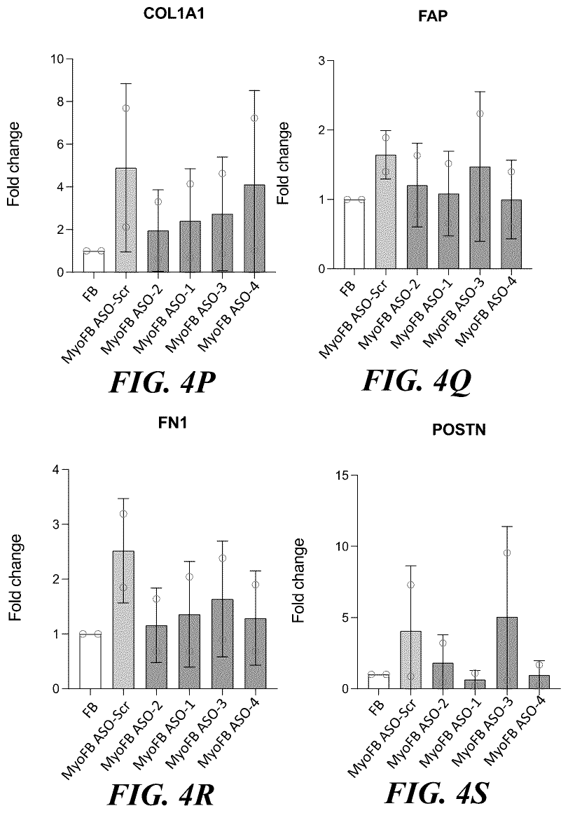

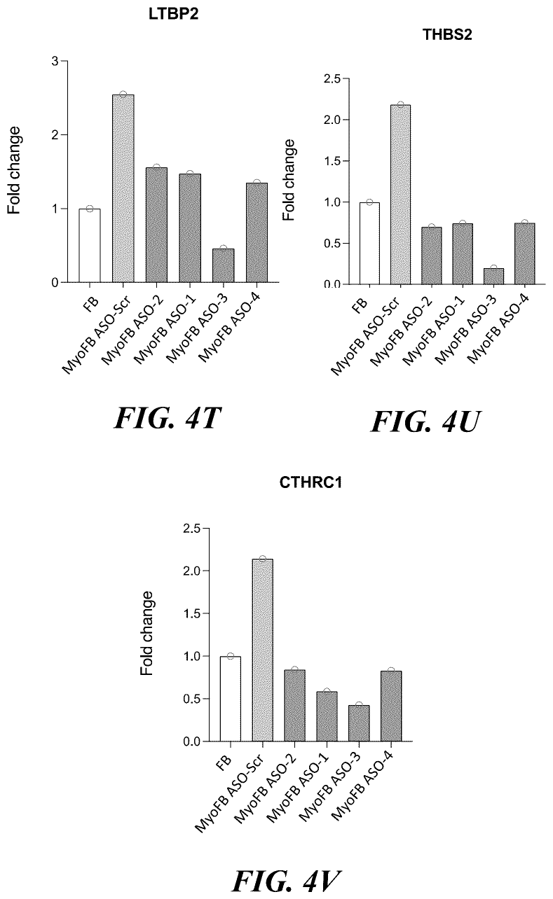

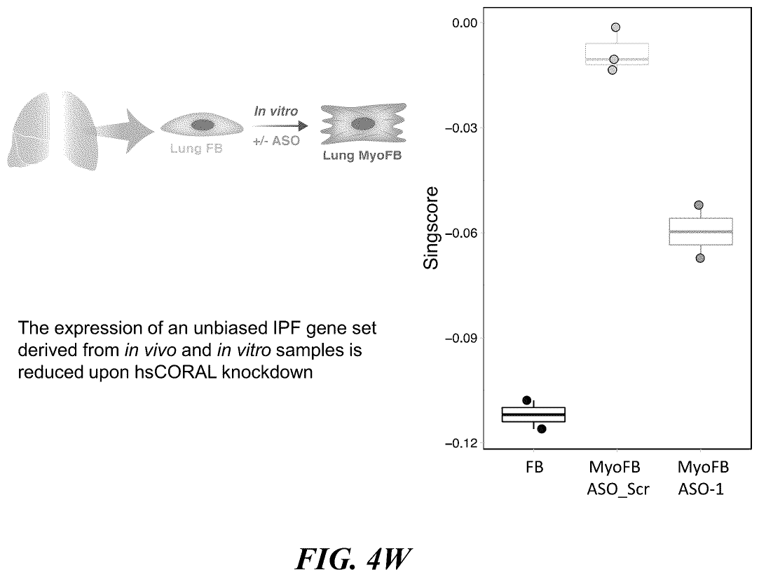

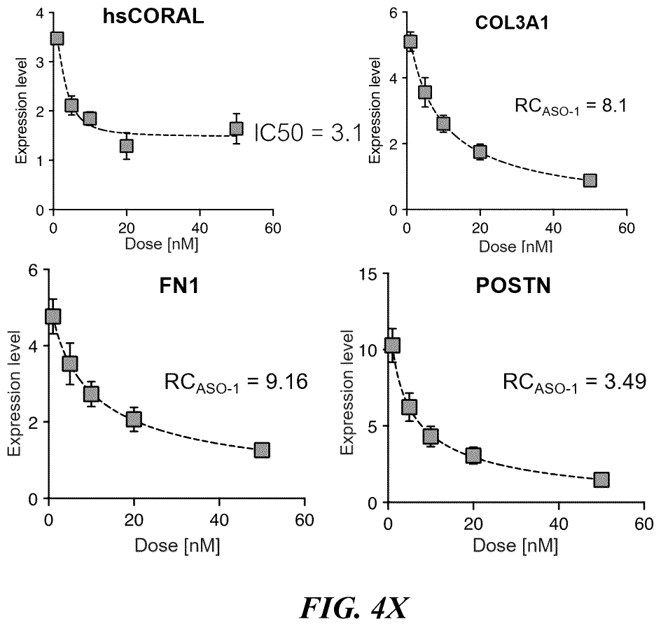

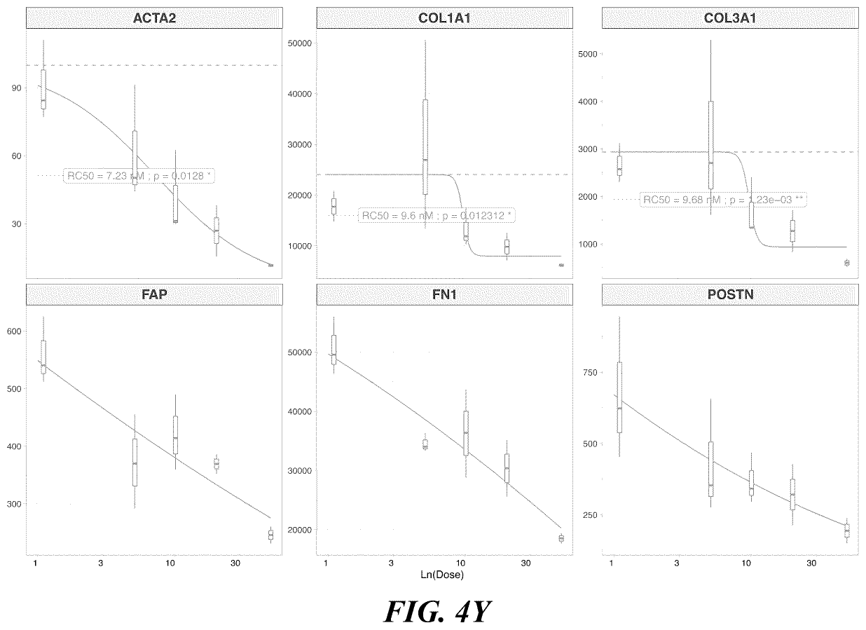

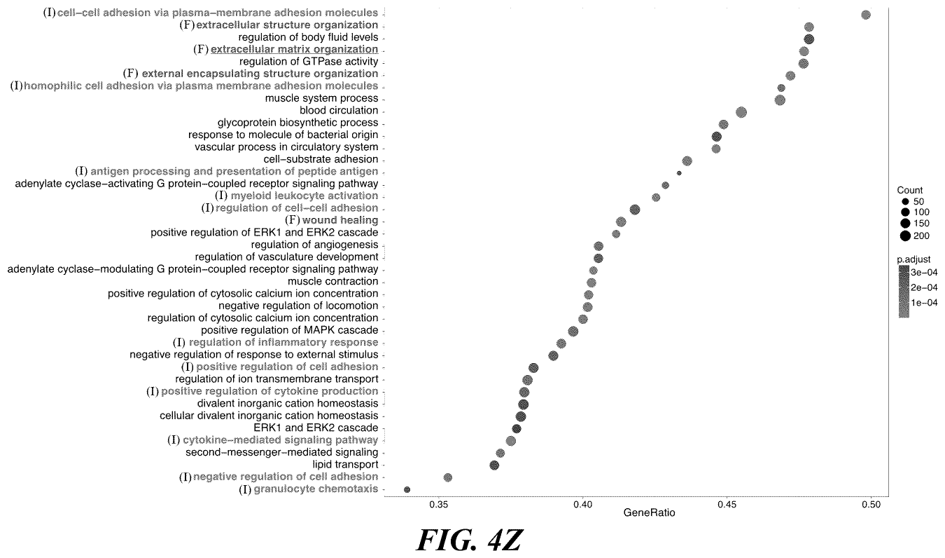

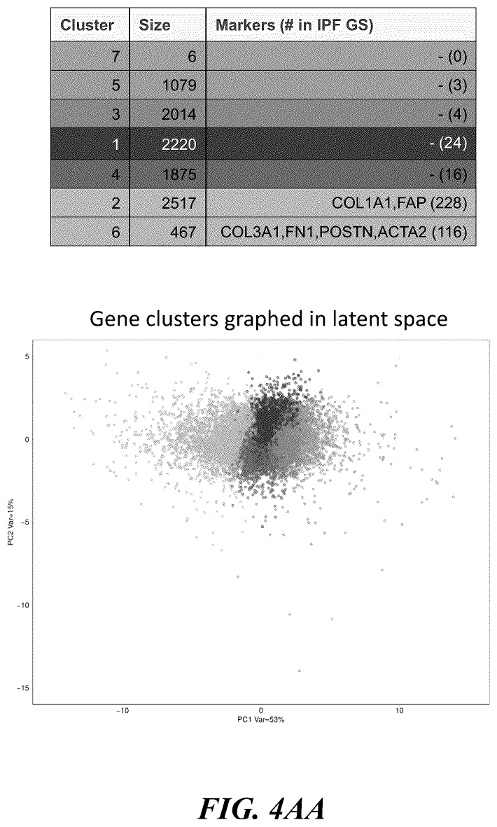

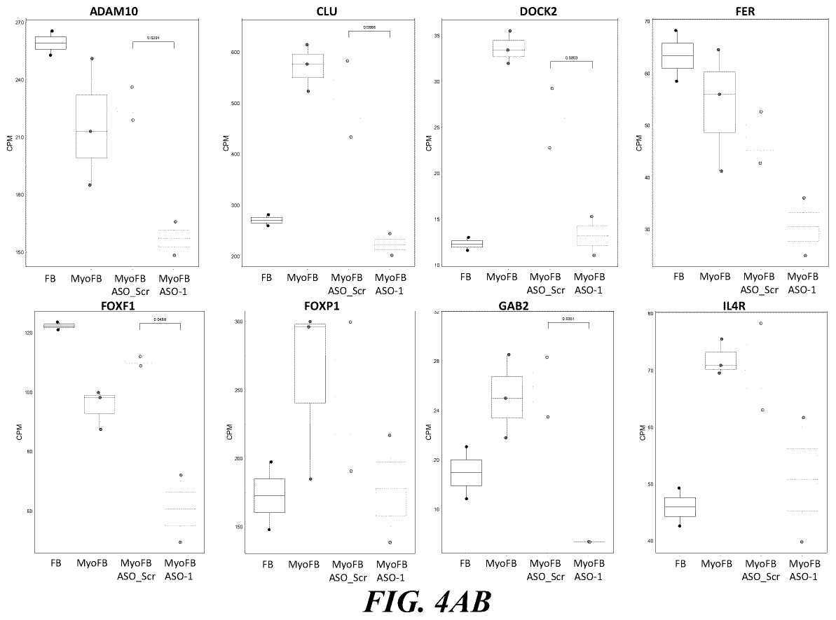

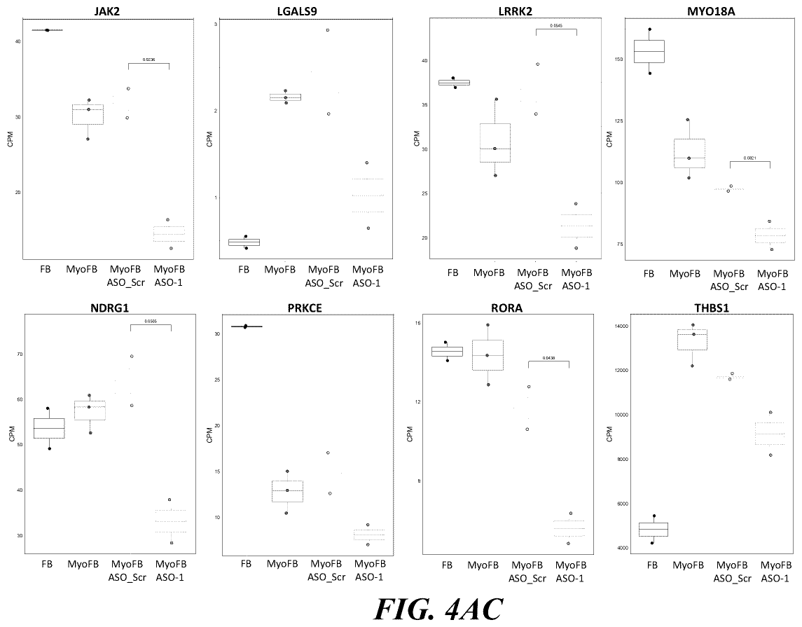

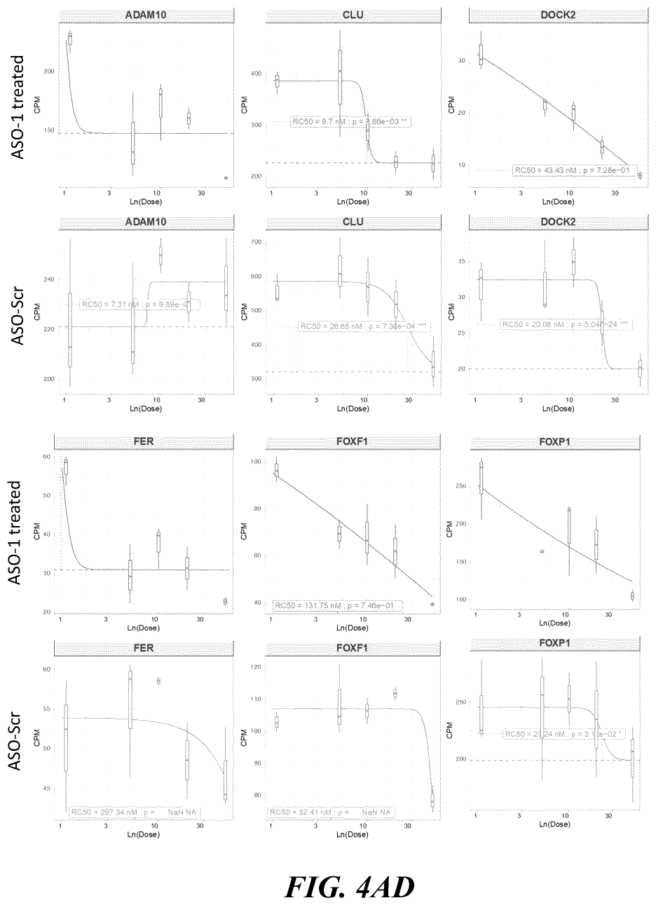

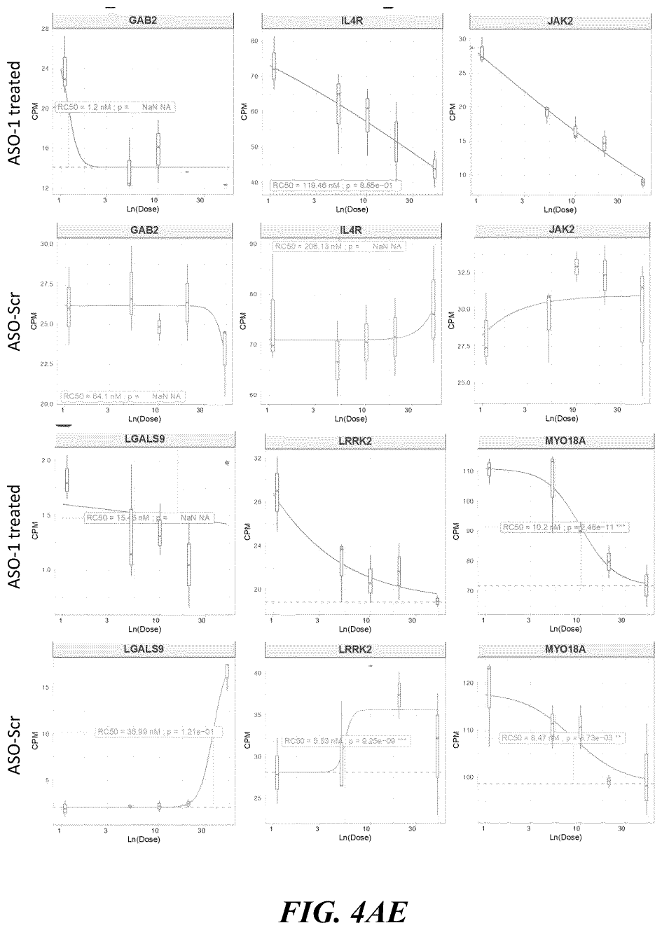

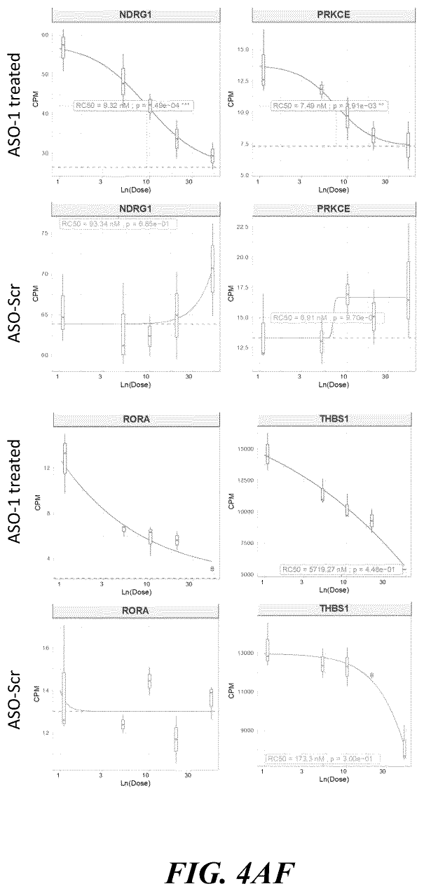

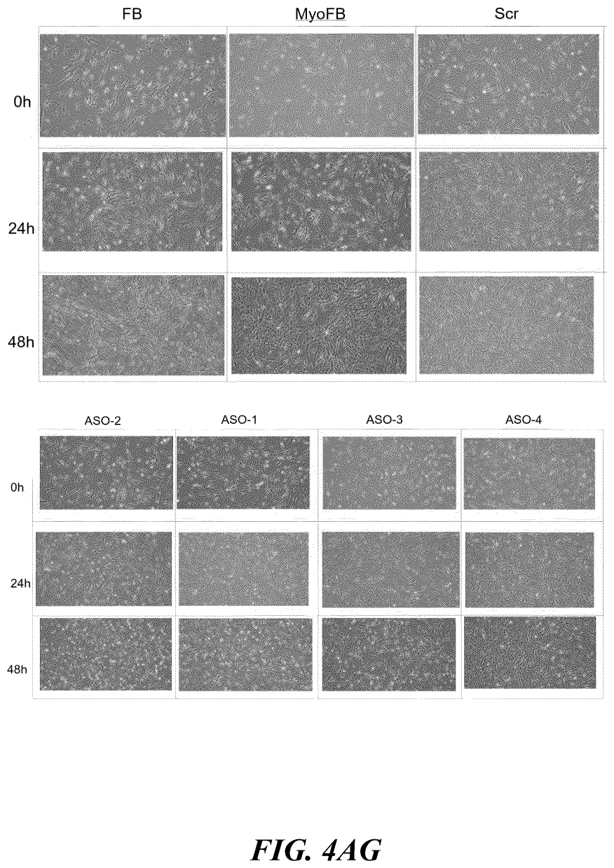

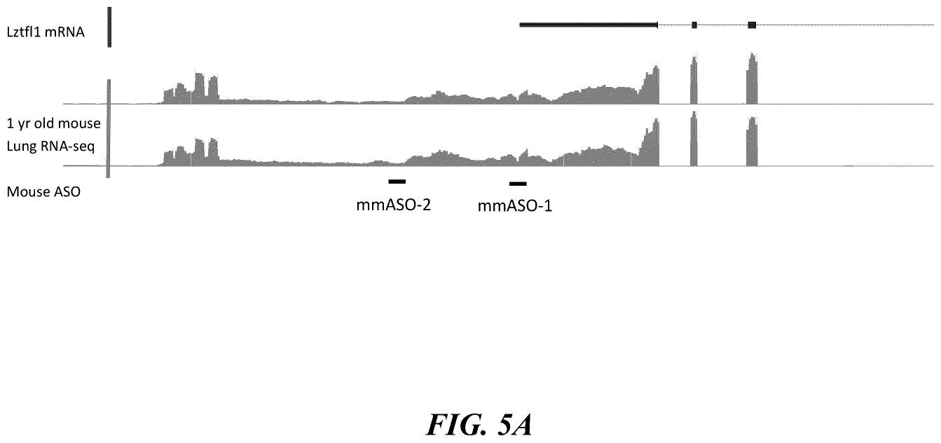

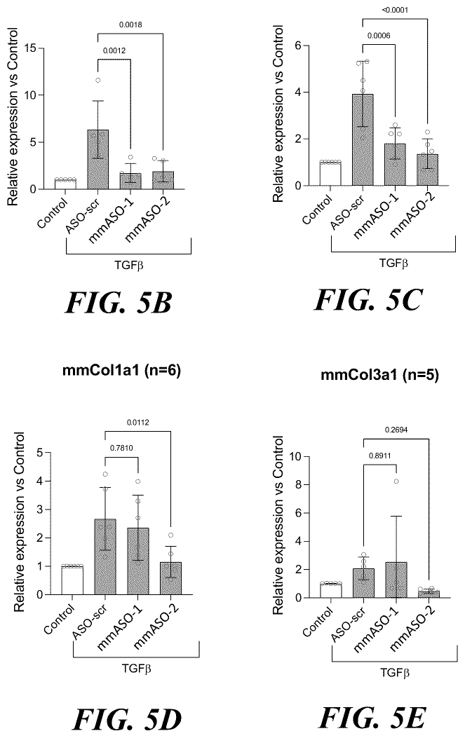

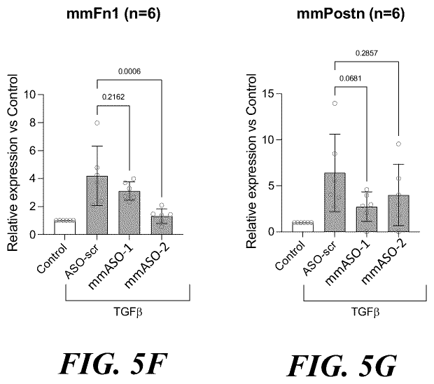

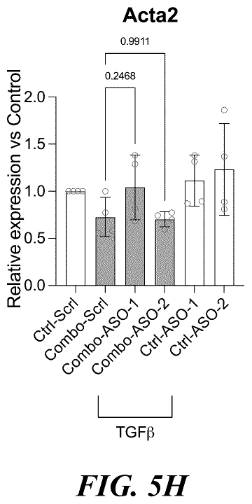

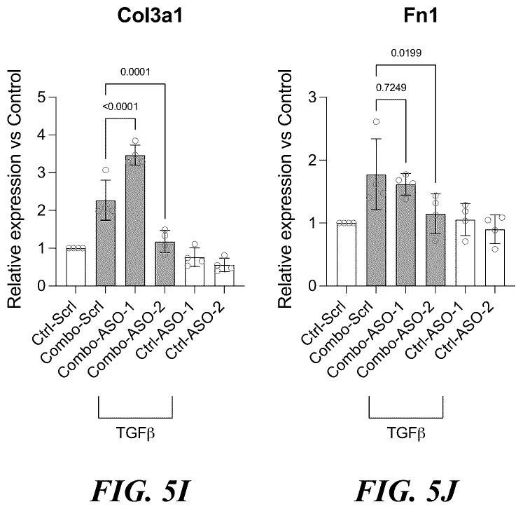

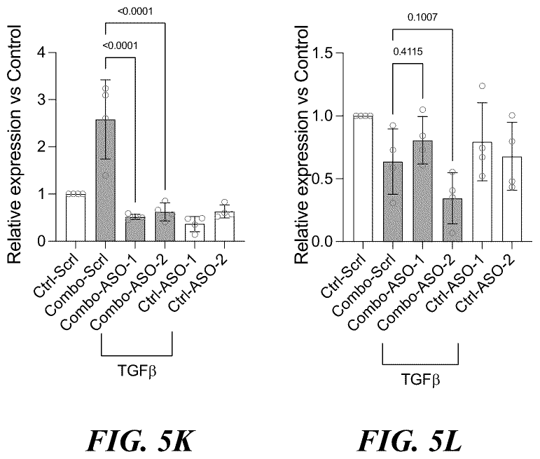

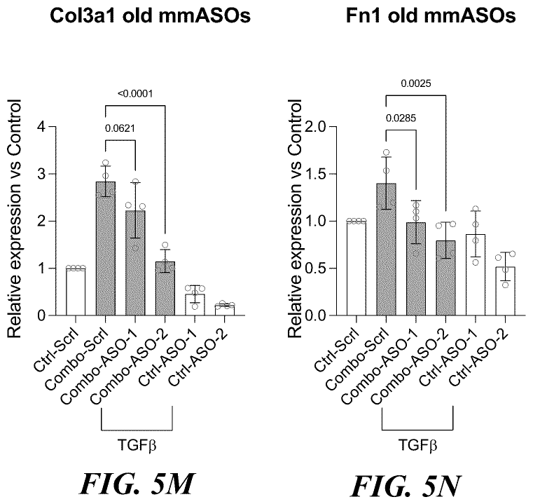

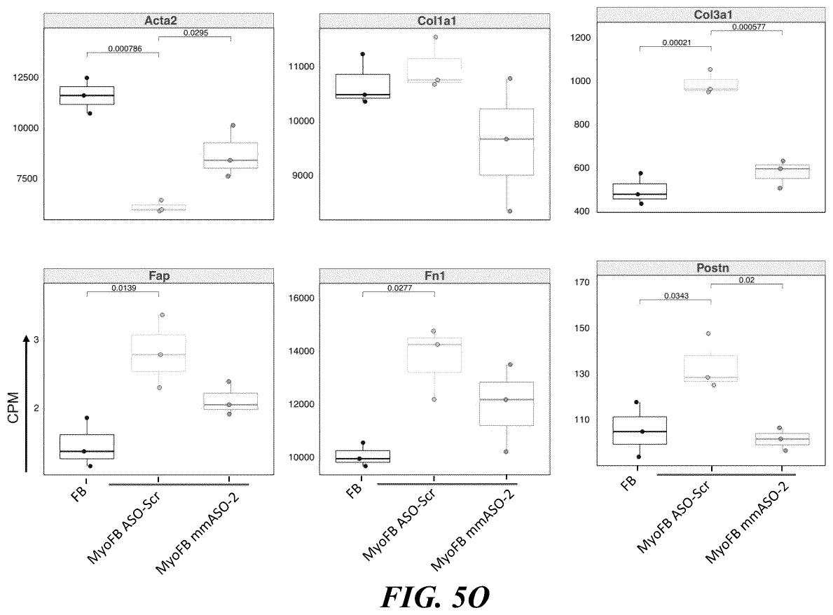

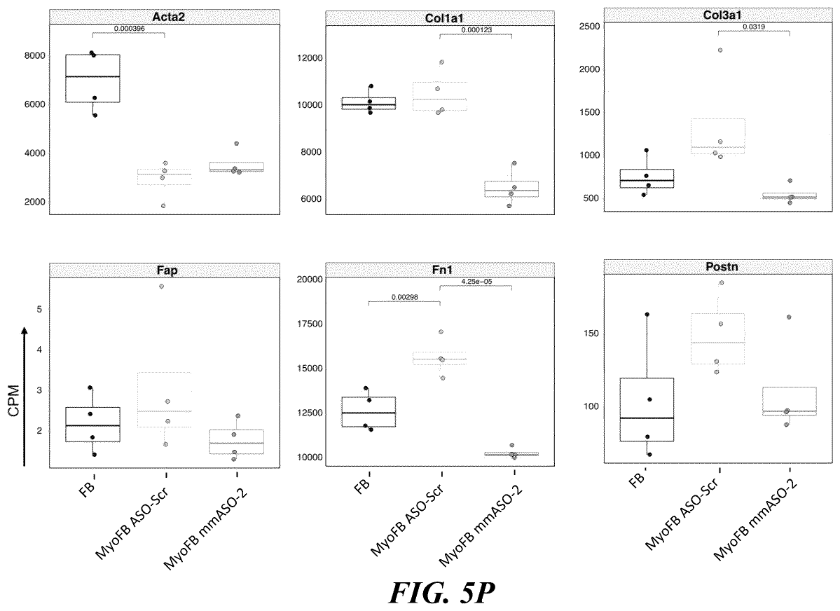

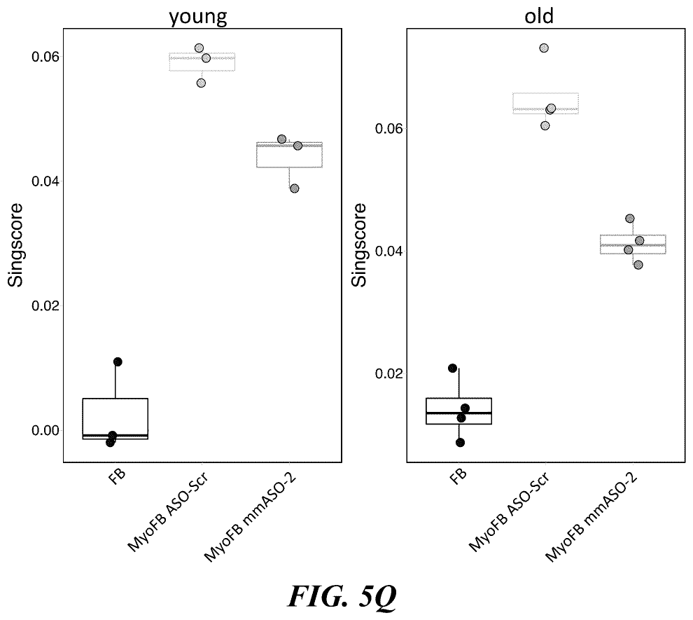

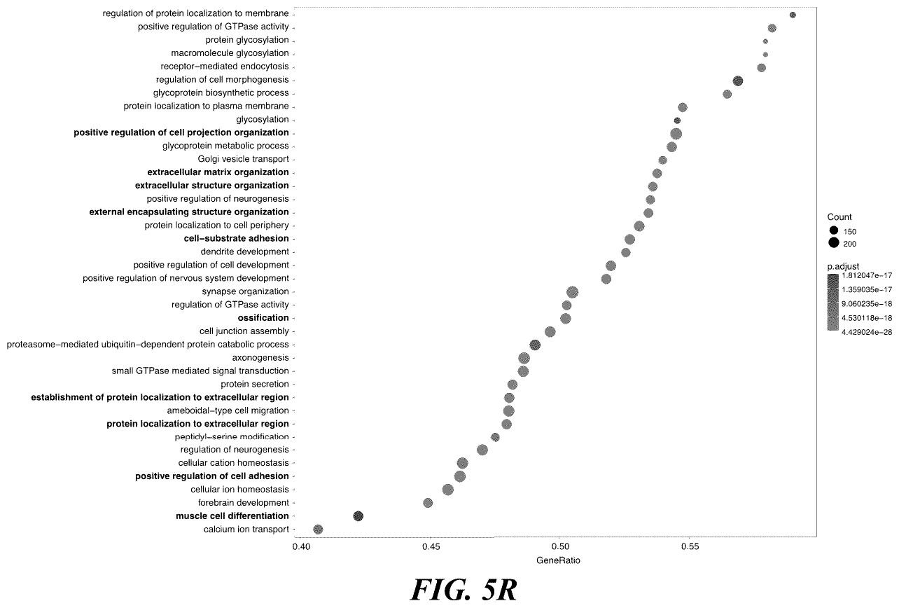

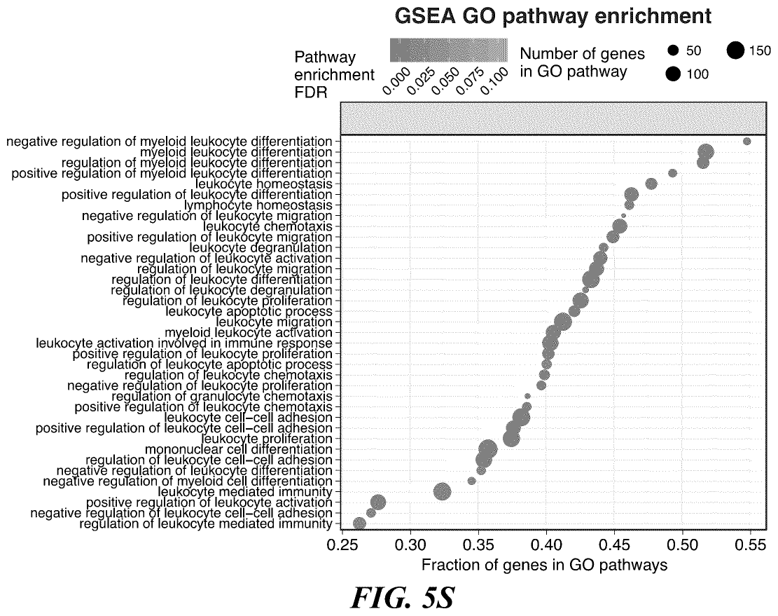

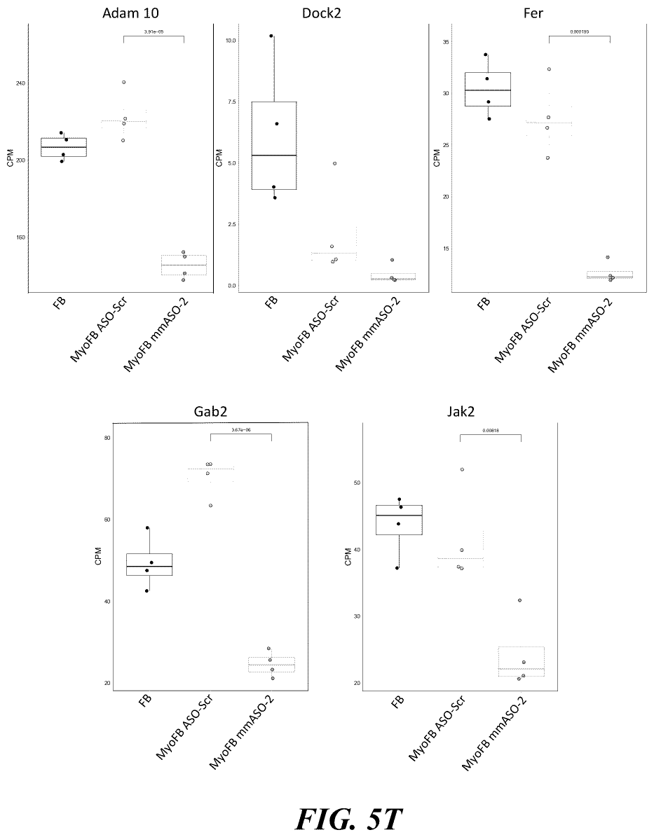

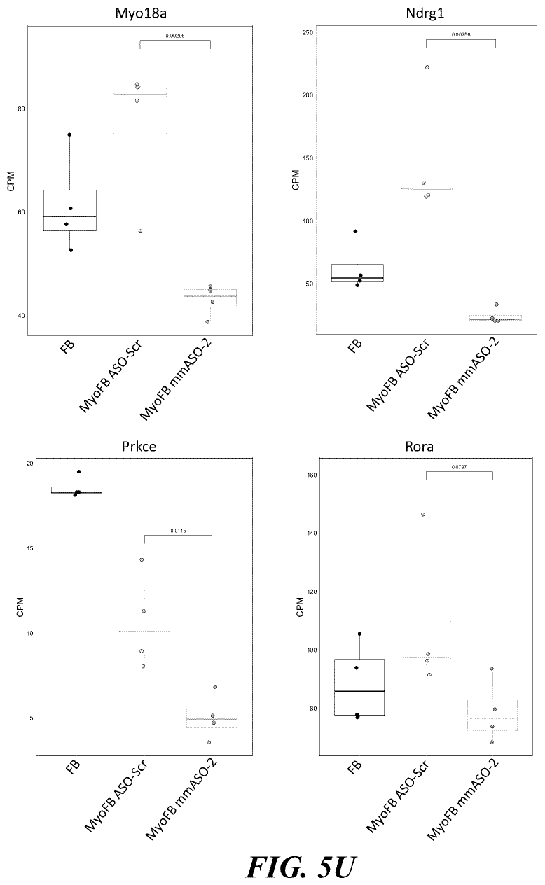

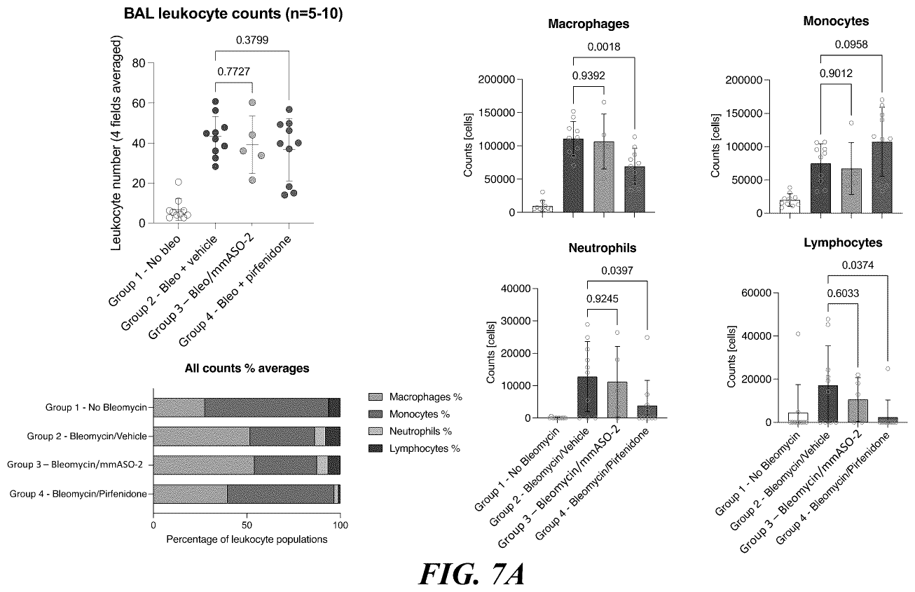

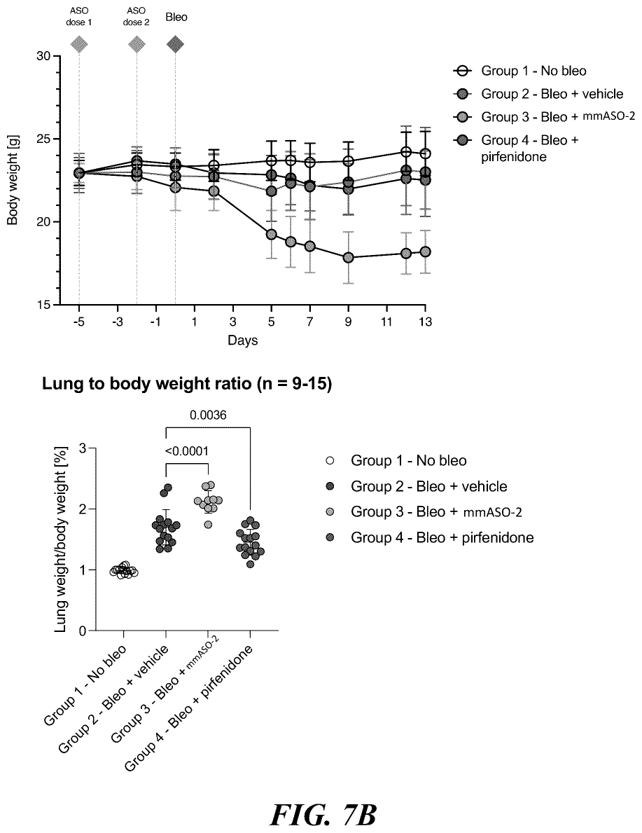

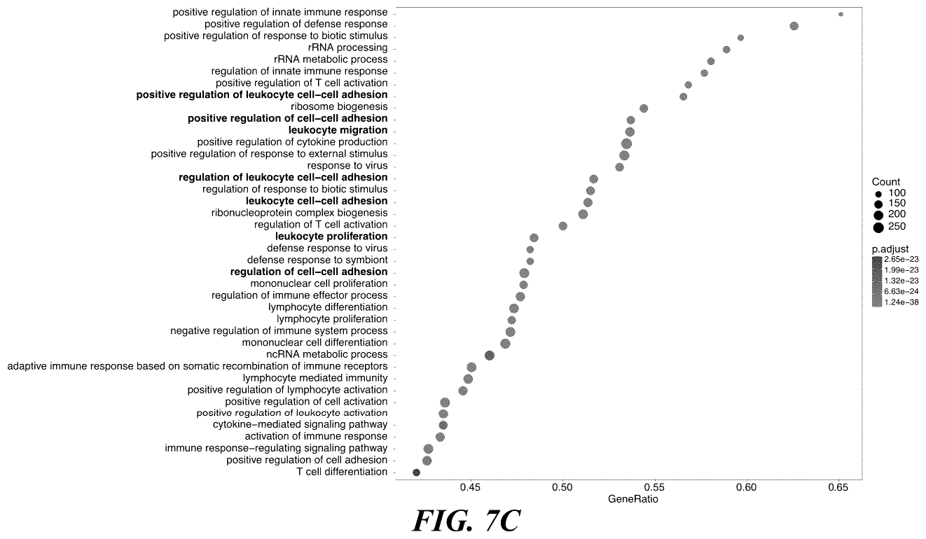

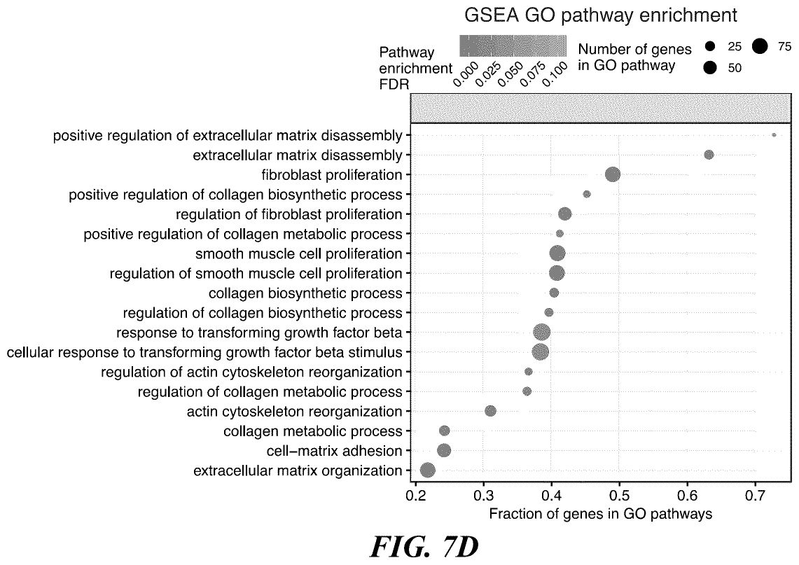

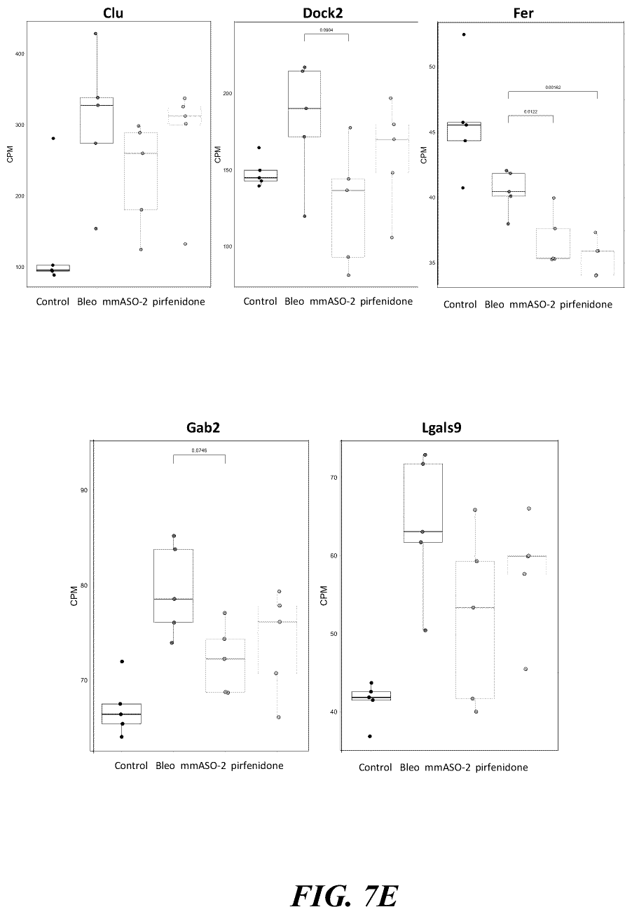

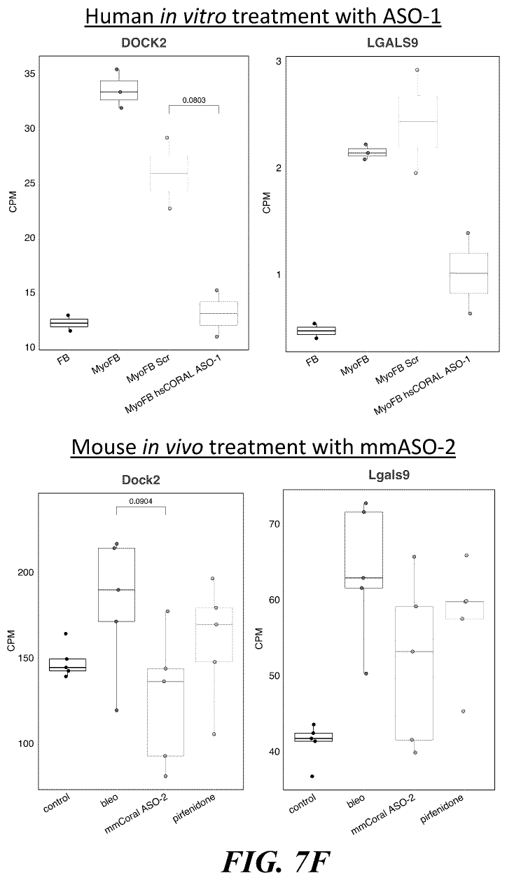

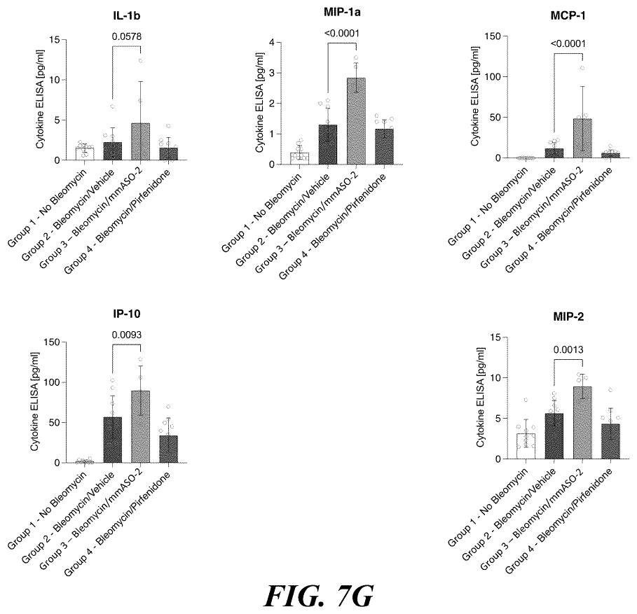

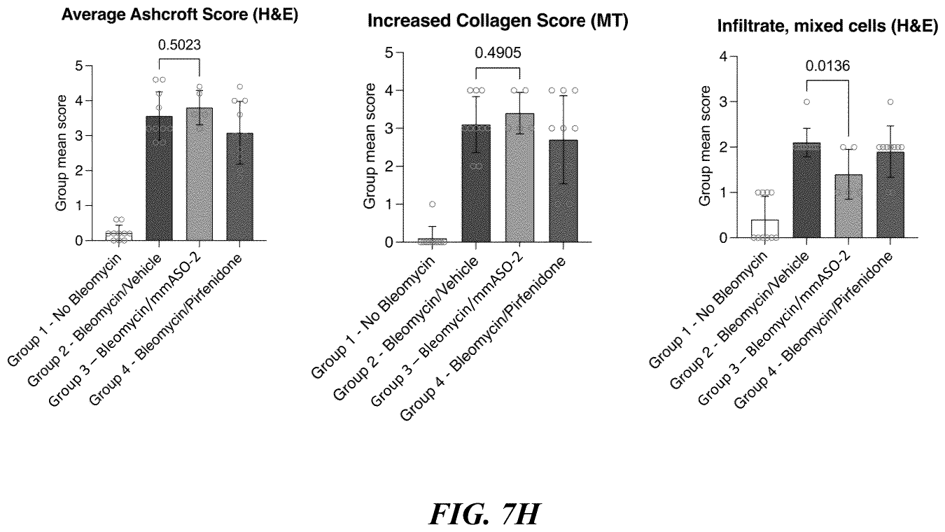

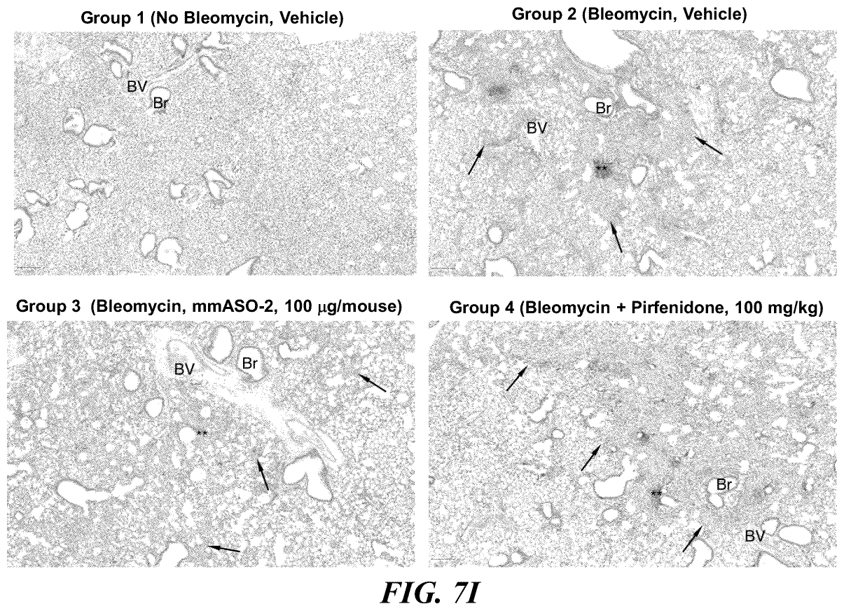

In some aspects, modulation of long noncoding transcript that is associated with onset, development, prognosis of a disease indication can be used to identify the druggable target for treating disease or can be used as a tool for diagnosis of the disease. As such, a modulator of the long noncoding transcript affects onset, development, prognosis of a disease indication. In some instances, a modulator of the long noncoding transcript affects onset, development, prognosis of pulmonary fibrosis associated with or induced by ARDS, e.g., ARDS associated with COVID-19 infection. In some instances, a modulator of the long noncoding transcript reduces symptoms or progress of pulmonary fibrosis associated with or induced by COVID-19 infection. In some instances, a modulator of the long noncoding transcript reduces intensity or severity of symptoms or progress of pulmonary fibrosis associated with or induced by ARDS, e.g., ARDS associated with COVID-19 infection. In some instances, a modulator of the long noncoding transcript reverses symptoms or progress of pulmonary fibrosis associated with or induced by ARDS associated with COVID-19 infection. In some instances, a modulator of the long noncoding transcript reduces ECM synthesis in the tissue affected by pulmonary fibrosis associated with or induced by ARDS, e.g., ARDS associated with COVID-19 infection, or in the tissue affected by COVID-19 infection. In some instances, a modulator of the long noncoding transcript increases or facilitate inflammation, inflammation symptoms, release of inflammation related cytokines or chemokines in the tissue affected by pulmonary fibrosis associated with or induced by ARDS, e.g., ARDS associated with COVID-19 infection, or in the tissue affected by COVID-19 infection. In some instances, a modulator of the long noncoding transcript increases or facilitate immune cell activation and/or immune cell infiltration in the tissue affected by pulmonary fibrosis associated with or induced by ARDS, e.g., ARDS associated with COVID-19 infection, or in the tissue affected by COVID-19 infection. In some instances, a modulator of the long noncoding transcript decreases or inhibits immune cell activation and/or immune cell infiltration in the tissue affected by pulmonary fibrosis associated with or induced by ARDS, e.g., ARDS associated with COVID-19 infection, or in the tissue affected by COVID-19 infection. In some instances, a modulator of the long noncoding transcript decreases or inhibits leukocyte infiltration, migration, and/or chemotaxis in the tissue affected by pulmonary fibrosis associated with or induced by ARDS, e.g., ARDS associated with COVID-19 infection, or in the tissue affected by COVID-19 infection. In some instances, a modulator of the long noncoding transcript decreases or inhibits granulocyte infiltration, migration, and/or chemotaxis in the tissue affected by pulmonary fibrosis associated with or induced by ARDS, e.g., ARDS associated with COVID-19 infection, or in the tissue affected by COVID-19 infection. In some instances, a modulator of the long noncoding transcript decreases or inhibits monocyte infiltration, migration, and/or chemotaxis in the tissue affected by pulmonary fibrosis associated with or induced by ARDS, e.g., ARDS associated with COVID-19 infection, or in the tissue affected by COVID-19 infection. In some instances, a modulator of the long noncoding transcript decreases or inhibits lymphocyte infiltration, migration, and/or chemotaxis in the tissue affected by pulmonary fibrosis associated with or induced by ARDS, e.g., ARDS associated with COVID-19 infection, or in the tissue affected by COVID-19 infection. In some instances, a modulator of the long noncoding transcript decreases or inhibits leukocyte activation in the tissue affected by pulmonary fibrosis associated with or induced by ARDS, e.g., ARDS associated with COVID-19 infection, or in the tissue affected by COVID-19 infection. In some instances, a modulator of the long noncoding transcript decreases or inhibits leukocyte cell-cell adhesion in the tissue affected by pulmonary fibrosis associated with or induced by ARDS, e.g., ARDS associated with COVID-19 infection, or in the tissue affected by COVID-19 infection. In some instances, a modulator of the long noncoding transcript decreases or inhibits leukocyte proliferation in the tissue affected by pulmonary fibrosis associated with or induced by ARDS, e.g., ARDS associated with COVID-19 infection, or in the tissue affected by COVID-19 infection. In some instances, a modulator of the long noncoding transcript decreases or inhibits leukocyte antigen processing and presentation in the tissue affected by pulmonary fibrosis associated with or induced by ARDS, e.g., ARDS associated with COVID-19 infection, or in the tissue affected by COVID-19 infection. In some instances, a modulator of the long noncoding transcript decreases or inhibits a leukocyte function sensitive to modulation by INFγ in the tissue affected by pulmonary fibrosis associated with or induced by ARDS, e.g., ARDS associated with COVID-19 infection, or in the tissue affected by COVID-19 infection. In some instances, a modulator of the long noncoding transcript decreases or inhibits T cell activation in the tissue affected by pulmonary fibrosis associated with or induced by ARDS, e.g., ARDS associated with COVID-19 infection, or in the tissue affected by COVID-19 infection. In some instances, a modulator of the long noncoding transcript decreases or inhibits one or more cellular functions listed by one or more GO terms in FIG. 4AG in the tissue affected by pulmonary fibrosis associated with or induced by ARDS, e.g., ARDS associated with COVID-19 infection, or in the tissue affected by COVID-19 infection. In some instances, the one or more GO terms comprises one or more fibrosis-related GO terms. In some instances, the one or more GO terms comprises one or more immune-related GO terms. In some instances, a modulator of the long noncoding transcript decreases or inhibits one or more cellular functions listed by one or more GO terms in FIG. 5R in the tissue affected by pulmonary fibrosis associated with or induced by ARDS, e.g., ARDS associated with COVID-19 infection, or in the tissue affected by COVID-19 infection. In some instances, the one or more GO terms comprises one or more extracellular matrix-related GO terms. In some instances, a modulator of the long noncoding transcript decreases or inhibits one or more cellular functions listed by one or more GO terms in FIG. 5S in the tissue affected by pulmonary fibrosis associated with or induced by ARDS, e.g., ARDS associated with COVID-19 infection, or in the tissue affected by COVID-19 infection. In some instances, the one or more GO terms comprises one or more leukocyte migration-related GO terms. In some instances, a modulator of the long noncoding transcript decreases or inhibits one or more cellular functions listed by one or more GO terms in FIG. 7C in the tissue affected by pulmonary fibrosis associated with or induced by ARDS, e.g., ARDS associated with COVID-19 infection, or in the tissue affected by COVID-19 infection. In some instances, the one or more GO terms comprises one or more GO terms related to leukocyte migration, leukocyte adhesion, leukocyte infiltration formation, or any combination thereof. In some instances, a modulator of the long noncoding transcript decreases or inhibits one or more cellular functions listed by one or more GO terms in FIG. 7D in the tissue affected by pulmonary fibrosis associated with or induced by ARDS, e.g., ARDS associated with COVID-19 infection, or in the tissue affected by COVID-19 infection. In some instances, the one or more GO terms comprises one or more extracellular matrix-related GO terms.

In some aspects, modulation of long noncoding transcript that is associated with onset, development, prognosis of a disease indication can regulate a transition in cell type. In some embodiments, the regulated transition in cell type is a transition from fibroblast to myofibroblast. In some embodiments, lung fibroblasts exhibit a higher rate or higher proportion of cells undergoing apoptosis. In some embodiments, lung fibroblasts do not exhibit a significant change in rate or proportion of cells undergoing apoptosis. In some embodiments, an extent of lung myofibroblast quiescence influences or correlates with a rate or a proportion of lung fibroblast cells undergoing apoptosis. In some embodiments, a greater extent of lung myofibroblast quiescence influences or correlates with a greater rate or a greater proportion of lung fibroblast cells undergoing apoptosis compared with lung tissue having a lesser extent of lung myofibroblast quiescence. In some embodiments, Annexin V and DAPI staining can be used to determine a rate or a proportion of lung fibroblast cells undergoing apoptosis. In some embodiments, Caspase 3/7 and LDD staining can be used to determine a rate or a proportion of lung fibroblast cells undergoing apoptosis. In some embodiments, treatment comprising administering an ASO targeting a lncRNA described herein does not significantly increase apoptosis in treated cells. In some embodiments, treatment comprising administering ASO-1 targeting hsCORAL does not significantly increase apoptosis in treated cells. In some embodiments, treatment comprising administering ASO-1 targeting hsCORAL does not significantly increase apoptosis in lung fibroblasts. In some embodiments, treatment comprising administering ASO-1 targeting hsCORAL to lung cells does not significantly increase apoptosis in lung fibroblasts compared with a treatment comprising administering a scrambled ASO to lung cells. In some embodiments, treatment comprising administering ASO-1 targeting hsCORAL to a subject does not significantly increase apoptosis in lung fibroblasts compared with a treatment comprising administering a scrambled ASO to a subject.

Types of Modulators

### (a) Gene Editing Moieties

In some aspects, the modulator disclosed herein is a nucleic acid editing or modifying tool. In some instances, the nucleic acid editing or modifying tool targets the genomic region disclosed herein. In some instances, the nucleic acid editing or modifying tool targets the long noncoding transcript. In some instances, the nucleic acid editing or modifying tool targets a premature form of the long noncoding transcript.

In some aspects, the nucleic acid editing or modifying tool is a programmable nucleic acid sequence specific endonuclease. In some instances, the nucleic acid editing or modifying tool is a nucleic acid guided endonuclease. In some instances, the nucleic acid editing or modifying tool is a CRISPR-based tool. In other instances, the nucleic acid editing or modifying tool is a meganuclease-based tool. In other instances, the nucleic acid editing or modifying tool is a zinc finger nuclease (ZFN)-based tool. In other aspects, the nucleic acid editing or modifying tool is a transcription activator-like effector-based nuclease (TALEN)-based tool. In other instances, the nucleic acid editing or modifying tool is an Argonaute system.

In some aspects, the CRISPR-based tool disclosed herein is a Type I, Type II, Type III, Type IV, Type V, or Type VI CRISPR system. CRISPR/Cas systems may be multi-protein systems or single effector protein systems. Multi-protein, or Class 1, CRISPR systems include Type I, Type III, and Type IV systems. In some instances, Class 2 systems include a single effector molecule and include Type II, Type V, and Type VI. In some aspects, the CRISPR-based tool disclosed herein comprises a single or multiple effector proteins. An effector protein may comprise one or multiple nuclease domains. An effector protein may target DNA or RNA, and the DNA or RNA may be single stranded or double stranded. Effector proteins may generate double strand or single strand breaks. Effector proteins may comprise mutations in a nuclease domain thereby generating a nickase protein. Effector proteins may comprise mutations in one or more nuclease domains, thereby generating a catalytically dead nuclease that is able to bind but not cleave a target sequence.

In some aspects, the CRISPR-based tool disclosed comprises a single or multiple guiding RNAs (gRNAs). In some aspects, the gRNA disclosed herein targets a portion of chr3:45,806,503 to chr3:45,834, 110. In some aspects, the gRNA disclosed herein targets a portion of chr3:45,806,503 to chr3:45,831,916. In some aspects, the gRNA disclosed herein targets a portion of chr3:45,796,552 to chr3: 45,834,110. In some aspects, the gRNA disclosed herein targets a portion of chr3:45,796,552 to chr3: 45,831,916. In some aspects, the gRNA disclosed herein targets a portion of chr3:45,796,552 to chr3: 45,829,603. In some aspects, the gRNA disclosed herein targets a portion of LOC107986083 (chr3: 45,817,379-45,827,511). The gRNA may comprise a crRNA. The gRNA may comprise a chimeric RNA with crRNA and tracrRNA sequences. The gRNA may comprise a separate crRNA and tracrRNA. Target nucleic acid sequences may comprise a protospacer adjacent motif (PAM) or a protospacer flanking site (PFS). The PAM or PFS may be 3′ or 5′ of the target or protospacer site. Cleavage of a target sequence may generate blunt ends, 3′ overhangs, or 5′ overhangs.

The gRNA disclosed herein may comprise a spacer sequence. Spacer sequences may be complementary to target sequences or protospacer sequences. Spacer sequences may be 10, 11, 12, 13, 14, 15, 16, 17, 18, 19, 20, 21, 22, 23, 24, 25, 26, 27, 28, 29, 30, 31, 32, 33, 34, 35, or 36 nucleotides in length. In some aspects, the spacer sequence may be less than 10 or more than 36 nucleotides in length.

The gRNA disclosed herein may comprise a repeat sequence. In some aspects, the repeat sequence is part of a double stranded portion of the gRNA. A repeat sequence may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some aspects, the spacer sequence may be less than 10 or more than 50 nucleotides in length.

The gRNA disclosed herein may comprise one or more synthetic nucleotides, non-naturally occurring nucleotides, nucleotides with a modification, deoxyribonucleotide, or any combination thereof. Additionally and/or alternatively, a gRNA may comprise a hairpin, linker region, single stranded region, double stranded region, or any combination thereof. Additionally or alternatively, a gRNA may comprise a signaling or reporter molecule.

The gRNA disclosed herein may be encoded by genetic or episomal DNA. The gRNA disclosed herein may be provided or delivered concomitantly with a CRISPR nuclease or sequentially. The gRNA disclosed herein may be chemically synthesized, in vitro transcribed or otherwise generated using standard RNA generation techniques known in the art.

The CRISPR-based tool disclosed herein can be a Type II CRISPR system, for example a Cas9 system. The Type II nuclease can comprise a single effector protein, which, In some aspects, comprises a RuvC and HNH nuclease domains. In some aspects, a functional Type II nuclease may comprise two or more polypeptides, each of which comprises a nuclease domain or fragment thereof. The target nucleic acid sequences may comprise a 3′ protospacer adjacent motif (PAM). In some aspects, the PAM may be 5′ of the target nucleic acid. Guide RNAs (gRNA) may comprise a single chimeric gRNA, which contains both crRNA and tracrRNA sequences. In some instances, the gRNA may comprise a set of two RNAs, for example a crRNA and a tracrRNA. The Type II nuclease may generate a double strand break, which in some cases creates two blunt ends. In some aspects, the Type II CRISPR nuclease is engineered to be a nickase such that the nuclease only generates a single strand break. In such cases, two distinct nucleic acid sequences may be targeted by gRNAs such that two single strand breaks are generated by the nickase. In some aspects, the two single strand breaks effectively create a double strand break. In some aspects where a Type II nickase is used to generate two single strand breaks, the resulting nucleic acid free ends may either be blunt, have a 3′ overhang, or a 5′ overhang. In some aspects, a Type II nuclease may be catalytically dead such that it binds to a target sequence, but does not cleave. For example, a Type II nuclease may have mutations in both the RuvC and HNH domains, thereby rendering the both nuclease domains non-functional.

A Type II CRISPR system may be one of three sub-types, namely Type II-A, Type II-B, or Type II-C.

The CRISPR-based tool disclosed herein can be a Type V CRISPR system, for example a Cpf1, C2cl, or C2c3 system. The Type V nuclease may comprise a single effector protein, which comprises a single RuvC nuclease domain. In other cases, a function Type V nuclease comprises a RuvC domain split between two or more polypeptides. In such cases, the target nucleic acid sequences may comprise a 5′ PAM or 3′ PAM. Guide RNAs (gRNA) may comprise a single gRNA or single crRNA, such as may be the case with Cpf1. In some aspects, a tracrRNA is not needed. In other examples, such as when C2cl is used, a gRNA may comprise a single chimeric gRNA, which contains both crRNA and tracrRNA sequences or the gRNA may comprise a set of two RNAs, for example a crRNA and a tracrRNA. The Type V CRISPR nuclease may generate a double strand break, which generates a 5′ overhang. In some aspects, the Type V CRISPR nuclease is engineered to be a nickase such that the nuclease only generates a single strand break. In such cases, two distinct nucleic acid sequences may be targeted by gRNAs such that two single strand breaks are generated by the nickase. In some aspects, the two single strand breaks effectively create a double strand break. In some aspects where a Type V nickase is used to generate two single strand breaks, the resulting nucleic acid free ends may either be blunt, have a 3′ overhang, or a 5′ overhang. In some aspects, a Type V nuclease may be catalytically dead such that it binds to a target sequence, but does not cleave. For example, a Type V nuclease may have mutations a RuvC domain, thereby rendering the nuclease domain non-functional.

The CRISPR-based tool disclosed herein may be a Type VI CRISPR system, for example a C2c2 system. A Type VI nuclease may comprise a HEPN domain. In some aspects, the Type VI nuclease comprises two or more polypeptides, each of which comprises a HEPN nuclease domain or fragment thereof. In such cases, the target nucleic acid sequences may by RNA, such as single stranded RNA. When using Type VI CRISPR system, a target nucleic acid may comprise a protospacer flanking site (PFS). The PFS may be 3′ or 5′ or the target or protospacer sequence. Guide RNAs (gRNA) may comprise a single gRNA or single crRNA. In some aspects, a tracrRNA is not needed. In other examples, a gRNA may comprise a single chimeric gRNA, which contains both crRNA and tracrRNA sequences or the gRNA may comprise a set of two RNAs, for example a crRNA and a tracrRNA. In some aspects, a Type VI nuclease may be catalytically dead such that it binds to a target sequence, but does not cleave. For example, a Type VI nuclease may have mutations in a HEPN domain, thereby rendering the nuclease domains non-functional.

Non-limiting examples of suitable nucleases, including nucleic acid-guided nucleases, for use in the present disclosure include C2cl, C2c2, C2c3, Cas1, Cas1B, Cas2, Cas3, Cas4, Cas5, Cas6, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csxl, Csx15, Csf1, Csf2, Csf3, Csf4, homologues thereof, orthologues thereof, or modified versions thereof.

In some aspects, The CRISPR-based tool disclosed herein is an Argonaute (Ago) system. Ago protein may be derived from a prokaryote, eukaryote, or archaea. The target nucleic acid may be RNA or DNA. A DNA target may be single stranded or double stranded. In some aspects, the target nucleic acid does not require a specific target flanking sequence, such as a sequence equivalent to a protospacer adjacent motif or protospacer flanking sequence. The Ago protein may create a double strand break or single strand break. In some aspects, when an Ago protein forms a single strand break, two Ago proteins may be used in combination to generate a double strand break. In some aspects, an Ago protein comprises one, two, or more nuclease domains. In some aspects, an Ago protein comprises one, two, or more catalytic domains. One or more nuclease or catalytic domains may be mutated in the Ago protein, thereby generating a nickase protein capable of generating single strand breaks. In other aspects, mutations in one or more nuclease or catalytic domains of an Ago protein generates a catalytically dead Ago protein that may bind but not cleave a target nucleic acid.

Ago proteins may be targeted to target nucleic acid sequences by a guiding nucleic acid. In some aspects, the guiding nucleic acid is a guide DNA (gDNA). The gDNA may have a 5′ phosphorylated end. The gDNA may be single stranded or double stranded. Single stranded gDNA may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some aspects, the gDNA may be less than 10 nucleotides in length. In some aspects, the gDNA may be more than 50 nucleotides in length.

Argonaute-mediated cleavage may generate blunt end, 5′ overhangs, or 3′ overhangs. In some aspects, one or more nucleotides are removed from the target site during or following cleavage.

In some aspects, the nucleic acid editing or modifying tool is a repressive dCas9 with the aid of a (single) guide RNA targeting the portion of the genomic region that is transcribed to the long noncoding transcript. In some instances, the nucleic acid editing or modifying tool is dCas9-KRAB-MECP2 with the aid of a (single) guide RNA targeting the portion of the genomic region that is transcribed to the long noncoding transcript. In other specific aspects, the nucleic acid editing or modifying tool is dCas9-KRAB-DNMT1 with the aid of a (single) guide RNA targeting the portion of the genomic region that is transcribed to the long noncoding transcript.

In the above-mentioned aspects, the (single) guide RNA targets 5′ side of an enhancer region the genomic region that is transcribed to the long noncoding transcript. In the certain aspects, the (single) guide RNA targets 5′ side of an enhancer region the genomic region that is transcribed to the long noncoding transcript.

### (b) Inhibitory Oligonucleotides

In some aspects, the modulator disclosed herein is a synthetic or artificial oligonucleotide or polynucleotide. In some instances, the oligonucleotide is a single-stranded nucleic acid molecule. In some instances, the single-stranded nucleic acid molecule comprises a nucleic acid sequence at least 9, 10 11, 12, 13, 14, 15 consecutive nucleotides that are complementary to at least a portion of a long noncoding transcript disclosed herein. In some instances, the single-stranded nucleic acid molecule comprises a nucleic acid sequence at least 80%, at least 85%, at least 90%, at least 95% complementary to at least a portion of a long noncoding transcript disclosed herein. In some instances, the oligonucleotide is a double-stranded nucleic acid molecule comprising a sense strand and an antisense strand. In some instances, the sense strand comprises a nucleic acid sequence at least 9, 10 11, 12, 13, 14, 15 consecutive nucleotides that are identical or at least 80%, at least 85%, at least 90%, at least 95% identical to at least a portion of a long noncoding transcript disclosed herein. In some instances, the antisense strand comprises a nucleic acid sequence at least 9, 10 11, 12, 13, 14, 15 consecutive nucleotides that are fully complementary or at least 80%, at least 85%, at least 90%, at least 95% complementary to at least a portion of a long noncoding transcript disclosed herein. In specific aspects, the synthetic or artificial oligonucleotide or polynucleotide is a small interfering RNA (siRNA), a microRNA (miRNA), an inhibitory double stranded RNA (dsRNA), a small or short hairpin RNA (shRNA), an antisense oligonucleotide (ASO), a phosphorodiamidate morpholino oligomer (PMO), a piwi-interacting RNA (piRNA), a heterogeneous nuclear RNA (hnRNA), a small nuclear RNA (snRNA), an enzymatically-prepared siRNA (esiRNA), or the precursors thereof.

In some aspects, the synthetic oligonucleotide disclosed herein is about 10-50 nucleotides long. In some instances, the synthetic oligonucleotide disclosed herein is about 10-40 nucleotides long. In some instances, the synthetic oligonucleotide disclosed herein is about 10-30, 10-28, 14-28, 14-25, 14-20, 15-25, or 18-25 nucleotides long. In other aspects, the synthetic oligonucleotide disclosed herein is at least 10 nucleotides long, at least 11 nucleotides long, at least 12 nucleotides long, at least 13 nucleotides long, at least 14 nucleotides long, at least 15 nucleotides long, or at least 16 nucleotides long. In other aspects, the synthetic oligonucleotide disclosed herein is at most 50 nucleotides long. In other aspects, the synthetic oligonucleotide disclosed herein is at most 40 nucleotides long. In other aspects, the synthetic oligonucleotide disclosed herein is at most 30 nucleotides long. In other aspects, the synthetic oligonucleotide disclosed herein is at most 20 nucleotides long. In other aspects, the synthetic oligonucleotide disclosed herein is about 15 nucleotides long. In other aspects, the synthetic oligonucleotide disclosed herein is about 16 nucleotides long. In other aspects, the synthetic oligonucleotide disclosed herein is about 17 nucleotides long. In other aspects, the synthetic oligonucleotide disclosed herein is 16 nucleotides long.

In some aspects, the synthetic oligonucleotide comprises one or more sugar modifications, one or more phosphate backbone modifications, one or more purine modifications, one or more pyrimidine modifications, or any combination thereof.

In some aspects, the sugar modifications disclosed herein comprises a modification at a 2′ hydroxyl group of the ribose moiety. In some instances, the modification includes an H, OR, R, halo, SH, SR, NH2, NHR, NR2, or CN, wherein R is an alkyl moiety. In some instances, the modification at the 2′ hydroxyl group is a 2′-O-methyl modification or a 2′-O-methoxyethyl (2′-O-MOE) modification, 2′-halo modification, 2′-fluoro modification, 2′-O-aminopropyl modification, locked or bridged ribose modification (e.g., locked nucleic acid (LNA)), ethylene nucleic acids (ENA), tricyclo-DNA, 2′ cyclic ethyl (cET), unlocked nucleic acid (UNA), and conformationally restricted nucleoside (CRN), or any combination thereof. In some aspects, the LNA disclosed herein comprises a beta-D-oxy LNA, an alpha-L-oxy-LNA, a beta-D-amino-LNA, an alpha-L-amino-LNA, a beta-D-thio-LNA, an alpha-L-thio-LNA, a 5′-methyl-LNA, a beta-D-ENA, or an alpha-L-ENA.

In some aspects, the one or more phosphate backbone modifications comprise phosphorothioate linkage, methylphosphonate linkage, guanidinopropyl phosphoramidate linkage, or any combination thereof.

In some aspects, the one or more purine modifications comprise 2,6-diaminopurin, 3-deaza-adenine, 7-deaza-guanine, 8-zaido-adenine, or any combination thereof.

In some aspects, the one or more pyrimidine modifications comprise 2-thio-thymidine, 5-carboxamide-uracil, 5-methyl-cytosine, 5-ethynyl-uracil, or any combination thereof.

In some aspects, the synthetic oligonucleotide comprises a phosphorodiamidate morpholino oligomer (PMO).

In some aspects, the synthetic oligonucleotide disclosed herein is an ASO. In some instances, the ASO comprises a gapmer comprising a central region of consecutive DNA nucleotides flanked by a 5′-wing region and 3′-wing region, wherein at least one of 5′-wing region and 3′-wing region comprises a nucleic acid analogue. In some instances, 5′-wing region comprises or consists of 2 or 3 nucleotides, RNA mimics, nucleic acid analogues, or combination thereof. In some instances, 3′-wing region comprises or consists of 2 or 3 nucleotides, RNA mimics, nucleic acid analogues, or combination thereof. In some instances, 5′-wing region comprises or consists of 3 nucleotides or nucleic acid analogues, or combination thereof, and 3′-wing region comprises or consists of 2 nucleotides or nucleic acid analogues, or combination thereof. In some instances, 5′-wing region comprises or consists of 2 nucleotides or nucleic acid analogues, or combination thereof, and 3′-wing region comprises or consists of 3 nucleotides or nucleic acid analogues, or combination thereof. In some instances, 5′-wing region comprises or consists of 3 nucleotides or nucleic acid analogues, or combination thereof, and 3′-wing region comprises or consists of 3 nucleotides or nucleic acid analogues, or combination thereof. In some instances, 5′-wing region comprises or consists of 3 nucleic acid analogues, and 3′-wing region comprises or consists of 2 nucleic acid analogues.

In some instance, the nucleic acid analogue comprises an LNA. In some aspects, the 5′-wing region of the gapmer disclosed herein comprises at least one LNA. In some aspects, the 5′-wing region of the gapmer disclosed herein comprises at least two LNAs. In some instances, the 5′-wing region of the gapmer disclosed herein comprises two consecutive LNAs. In some aspects, the 5′-wing region of the gapmer disclosed herein comprises at least three LNAs. In some specific aspects, the 5′-wing region of the gapmer disclosed herein comprises three consecutive LNAs. In some aspects, the 5′-wing region of the gapmer disclosed herein comprises at least four LNAs. In some specific aspects, the 5′-wing region of the gapmer disclosed herein comprises four consecutive LNAs.

In some aspects, the 3′-wing region comprises an LNA. In some aspects, the 3′-wing region comprises at least two LNAs. In specific aspects, the 3′-wing region comprises two consecutive LNAs. In some aspects, the 3′-wing region comprises at least three LNAs. In specific aspects, the 3′-wing region comprises three consecutive LNAs.

In some aspects, the synthetic oligonucleotide disclosed herein comprises at least 8, at least 9, at least 10, at least 11, at least 12, at least 13, at least 14, at least 15 consecutive nucleotides from SEQ ID NO: 1 from Table 3. In some instances, the synthetic oligonucleotide disclosed herein comprises at least 8 consecutive nucleotides with no more than 8 mismatches, no more than 7 mismatches, no more than 6 mismatches, no more than 5 mismatches, no more than 4 mismatches, no more than 3 mismatches, no more than 2 mismatches or no more than 1 mismatch from SEQ ID NO: 1 from Table 3. In some instances, the synthetic oligonucleotide disclosed herein comprises at least 9 consecutive nucleotides with no more than 7 mismatches, no more than 6 mismatches, no more than 5 mismatches, no more than 4 mismatches, no more than 3 mismatches, no more than 2 mismatches or no more than 1 mismatch from SEQ ID NO: 1 from Table 3. In some instances, the synthetic oligonucleotide disclosed herein comprises at least 10 consecutive nucleotides with no more than 6 mismatches, no more than 5 mismatches, no more than 4 mismatches, no more than 3 mismatches, no more than 2 mismatches or no more than 1 mismatch from SEQ ID NO: 1 from Table 3. In some instances, the synthetic oligonucleotide disclosed herein comprises at least 11 consecutive nucleotides with no more than 5 mismatches, no more than 4 mismatches, no more than 3 mismatches, no more than 2 mismatches or no more than 1 mismatch from SEQ ID NO: 1 from Table 3. In some instances, the synthetic oligonucleotide disclosed herein comprises at least 12 consecutive nucleotides with no more than 4 mismatches, no more than 3 mismatches, no more than 2 mismatches or no more than 1 mismatch from SEQ ID NO: 1 from Table 3. In some instances, the synthetic oligonucleotide disclosed herein comprises at least 13 consecutive nucleotides with no more than 3 mismatches, no more than 2 mismatches or no more than 1 mismatch from SEQ ID NO: 1 from Table 3. In some instances, the synthetic oligonucleotide disclosed herein comprises at least 14 consecutive nucleotides with no more than 2 mismatches or no more than 1 mismatch from SEQ ID NO: 1 from Table 3. In some instances, the synthetic oligonucleotide disclosed herein comprises 15 consecutive nucleotides with 1 mismatch from SEQ ID NO: 1 from Table 3.

In some aspects, the synthetic oligonucleotide disclosed herein comprises at least 8, at least 9, at least 10, at least 11, at least 12, at least 13, at least 14, at least 15 consecutive nucleotides from SEQ ID NO: 2 from Table 3. In some instances, the synthetic oligonucleotide disclosed herein comprises at least 8 consecutive nucleotides with no more than 8 mismatches, no more than 7 mismatches, no more than 6 mismatches, no more than 5 mismatches, no more than 4 mismatches, no more than 3 mismatches, no more than 2 mismatches or no more than 1 mismatch from SEQ ID NO: 2 from Table 3. In some instances, the synthetic oligonucleotide disclosed herein comprises at least 9 consecutive nucleotides with no more than 7 mismatches, no more than 6 mismatches, no more than 5 mismatches, no more than 4 mismatches, no more than 3 mismatches, no more than 2 mismatches or no more than 1 mismatch from SEQ ID NO: 2 from Table 3. In some instances, the synthetic oligonucleotide disclosed herein comprises at least 10 consecutive nucleotides with no more than 6 mismatches, no more than 5 mismatches, no more than 4 mismatches, no more than 3 mismatches, no more than 2 mismatches or no more than 1 mismatch from SEQ ID NO: 2 from Table 3. In some instances, the synthetic oligonucleotide disclosed herein comprises at least 11 consecutive nucleotides with no more than 5 mismatches, no more than 4 mismatches, no more than 3 mismatches, no more than 2 mismatches or no more than 1 mismatch from SEQ ID NO: 2 from Table 3. In some instances, the synthetic oligonucleotide disclosed herein comprises at least 12 consecutive nucleotides with no more than 4 mismatches, no more than 3 mismatches, no more than 2 mismatches or no more than 1 mismatch from SEQ ID NO: 2 from Table 3. In some instances, the synthetic oligonucleotide disclosed herein comprises at least 13 consecutive nucleotides with no more than 3 mismatches, no more than 2 mismatches or no more than 1 mismatch from SEQ ID NO: 2 from Table 3. In other aspects, the synthetic oligonucleotide disclosed herein comprises at least 14 consecutive nucleotides with no more than 2 mismatches or no more than 1 mismatch from SEQ ID NO: 2 from Table 3. In some instances, the synthetic oligonucleotide disclosed herein comprises 15 consecutive nucleotides with 1 mismatch from SEQ ID NO: 2 from Table 3.

In some instances, the synthetic oligonucleotide disclosed herein comprises at least 8, at least 9, at least 10, at least 11, at least 12, at least 13, at least 14, at least 15 consecutive nucleotides from SEQ ID NO: 3 from Table 3. In some instances, the synthetic oligonucleotide disclosed herein comprises at least 8 consecutive nucleotides with no more than 8 mismatches, no more than 7 mismatches, no more than 6 mismatches, no more than 5 mismatches, no more than 4 mismatches, no more than 3 mismatches, no more than 2 mismatches or no more than 1 mismatch from SEQ ID NO: 3 from Table 3. In some instances, the synthetic oligonucleotide disclosed herein comprises at least 9 consecutive nucleotides with no more than 7 mismatches, no more than 6 mismatches, no more than 5 mismatches, no more than 4 mismatches, no more than 3 mismatches, no more than 2 mismatches or no more than 1 mismatch from SEQ ID NO: 3 from Table 3. In some instances, the synthetic oligonucleotide disclosed herein comprises at least 10 consecutive nucleotides with no more than 6 mismatches, no more than 5 mismatches, no more than 4 mismatches, no more than 3 mismatches, no more than 2 mismatches or no more than 1 mismatch from SEQ ID NO: 3 from Table 3. In some instances, the synthetic oligonucleotide disclosed herein comprises at least 11 consecutive nucleotides with no more than 5 mismatches, no more than 4 mismatches, no more than 3 mismatches, no more than 2 mismatches or no more than 1 mismatch from SEQ ID NO: 3 from Table 3. In some instances, the synthetic oligonucleotide disclosed herein comprises at least 12 consecutive nucleotides with no more than 4 mismatches, no more than 3 mismatches, no more than 2 mismatches or no more than 1 mismatch from SEQ ID NO: 3 from Table 3. In some instances, the synthetic oligonucleotide disclosed herein comprises at least 13 consecutive nucleotides with no more than 3 mismatches, no more than 2 mismatches or no more than 1 mismatch from SEQ ID NO: 3 from Table 3. In some instances, the synthetic oligonucleotide disclosed herein comprises at least 14 consecutive nucleotides with no more than 2 mismatches or no more than 1 mismatch from SEQ ID NO: 3 from Table 3. In some instances, the synthetic oligonucleotide disclosed herein comprises 15 consecutive nucleotides with 1 mismatch from SEQ ID NO: 3 from Table 3.

In some aspects, the synthetic oligonucleotide disclosed herein comprises at least 8, at least 9, at least 10, at least 11, at least 12, at least 13, at least 14, at least 15 consecutive nucleotides from SEQ ID NO: 4 from Table 3. In some instances, the synthetic oligonucleotide disclosed herein comprises at least 8 consecutive nucleotides with no more than 8 mismatches, no more than 7 mismatches, no more than 6 mismatches, no more than 5 mismatches, no more than 4 mismatches, no more than 3 mismatches, no more than 2 mismatches or no more than 1 mismatch from SEQ ID NO: 4 from Table 3. In some instances, the synthetic oligonucleotide disclosed herein comprises at least 9 consecutive nucleotides with no more than 7 mismatches, no more than 6 mismatches, no more than 5 mismatches, no more than 4 mismatches, no more than 3 mismatches, no more than 2 mismatches or no more than 1 mismatch from SEQ ID NO: 4 from Table 3. In some instances, the synthetic oligonucleotide disclosed herein comprises at least 10 consecutive nucleotides with no more than 6 mismatches, no more than 5 mismatches, no more than 4 mismatches, no more than 3 mismatches, no more than 2 mismatches or no more than 1 mismatch from SEQ ID NO: 4 from Table 3. In v, the synthetic oligonucleotide disclosed herein comprises at least 11 consecutive nucleotides with no more than 5 mismatches, no more than 4 mismatches, no more than 3 mismatches, no more than 2 mismatches or no more than 1 mismatch from SEQ ID NO: 4 from Table 3. In some instances, the synthetic oligonucleotide disclosed herein comprises at least 12 consecutive nucleotides with no more than 4 mismatches, no more than 3 mismatches, no more than 2 mismatches or no more than 1 mismatch from SEQ ID NO: 4 from Table 3. In some instances, the synthetic oligonucleotide disclosed herein comprises at least 13 consecutive nucleotides with no more than 3 mismatches, no more than 2 mismatches or no more than 1 mismatch from SEQ ID NO: 4 from Table 3. In some instances, the synthetic oligonucleotide disclosed herein comprises at least 14 consecutive nucleotides with no more than 2 mismatches or no more than 1 mismatch from SEQ ID NO: 4 from Table 3. In some instances, the synthetic oligonucleotide disclosed herein comprises 15 consecutive nucleotides with 1 mismatch from SEQ ID NO: 4 from Table 3.

In some aspects, the synthetic oligonucleotide disclosed herein comprises a nucleic acid sequence at least 80%, at least 85%, at least 90%, at least 95%, at least 96%, at least 97%, at least 98%, at least 99% identical to a sequence selected from SEQ ID NO: 1 from Table 3. In some aspects, the synthetic oligonucleotide disclosed herein comprises a nucleic acid sequence at least 80%, at least 85%, at least 90%, at least 95%, at least 96%, at least 97%, at least 98%, at least 99% identical to a sequence selected from SEQ ID NO: 2 from Table 3. In some aspects, the synthetic oligonucleotide disclosed herein comprises a nucleic acid sequence at least 80%, at least 85%, at least 90%, at least 95%, at least 96%, at least 97%, at least 98%, at least 99% identical to a sequence selected from SEQ ID NO: 3 from Table 3. In some aspects, the synthetic oligonucleotide disclosed herein comprises a nucleic acid sequence at least 80%, at least 85%, at least 90%, at least 95%, at least 96%, at least 97%, at least 98%, at least 99% identical to a sequence selected from SEQ ID NO: 4 from Table 3.

### (c) Pharmaceutical Compositions and Kits

Further provided herein are pharmaceutical compositions comprising the modulator disclosed herein and a pharmaceutically acceptable salt, excipient, or derivative thereof.

The suitable pharmaceutically acceptable salts or derivative thereof include but are not limited to (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, and the like; and (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like.

A pharmaceutical composition described herein can be prepared to include the modulator disclosed herein, into a form suitable for administration to a subject using carriers, excipients, and vehicles. In some instances, excipients include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases.

Other pharmaceutically acceptable vehicles include aqueous solutions, non-toxic excipients, including salts, preservatives, buffers and the like, as described, for instance, in Remington: The Science and Practice of Pharmacy, 21st Ed., Gennaro, Ed., Lippencott Williams & Wilkins (2005), and The United States Pharmacopeia: The National Formulary (USP 36 NF31), published in 2013. The pH and exact concentration of the various components of the pharmaceutical composition are adjusted according to routine skills in the art. See Goodman and Gilman's, The Pharmacological Basis for Therapeutics.

The pharmaceutical compositions described herein may be administered locally or systemically. The therapeutically effective amounts will vary according to factors, such as the degree of infection in a subject, the age, sex, health conditions, and weight of the individual.

Dosage regimes can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, intraorbital, and the like), oral administration, ophthalmic application, inhalation, topical application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition can be sterile and fluid to the extent that easy syringability exists. The composition can be stable under the conditions of manufacture and storage and preserved against the contaminating action of microorganisms, such as bacteria and fungi. The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of certain particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in an appropriate solvent with one or a combination of ingredients enumerated above followed by filtered sterilization. Generally, dispersions are prepared by incorporating the pharmaceutical composition into a sterile vehicle that contains a basic dispersion medium and the other ingredients from those enumerated above.

It is advantageous to formulate parenteral compositions in dosage unit form for ease of administration and uniformity of dosage. Dosage unit form as used herein, refers to physically discrete units suited as unitary dosages for the subject to be treated; each unit containing a predetermined quantity of pharmaceutical composition is calculated to produce the desired therapeutic effect in association with the pharmaceutical vehicle. The specification for the dosage unit forms are related to the characteristics of the pharmaceutical composition and the particular therapeutic effect to be achieve. The principal pharmaceutical composition is compounded for convenient and effective administration in effective amounts with a suitable pharmaceutically acceptable vehicle in an acceptable dosage unit. In the case of compositions containing supplementary active ingredients, the dosages are determined by reference to the usual dose and manner of administration of the ingredients.

The pharmaceutical composition can be orally administered, for example, in a carrier, e.g., in an enteric-coated unit dosage form. The pharmaceutical composition and other ingredients can also be enclosed in a hard or soft-shell gelatin capsule or compressed into tablets.

For oral therapeutic administration, the pharmaceutical composition can be incorporated with excipients and used in the form of ingestible tablets, troches, capsules, pills, wafers, and the like.

Such compositions and preparations may contain at least 1% by weight of active compound. The percentage of the compositions and preparations can, of course, be varied and can conveniently be between about 5% to about 80% of the weight of the unit. The tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Any material used in preparing any dosage unit form can be of pharmaceutically acceptable purity and substantially non-toxic in the amounts employed. In addition, the pharmaceutical composition can be incorporated into sustained-release preparations and formulations.

The pharmaceutical composition described herein may comprise one or more permeation enhancer that facilitates bioavailability of the modulator described herein. WO 2000/67798, Muranishi, 1990, Crit. Rev. Ther. Drug Carrier Systems, 7, 1, Lee et al., 1991, Crit. Rev. Ther. Drug Carrier Systems, 8, 91 are herein incorporated by reference in its entirety. In some aspects, the permeation enhancer is intestinal. In some aspects, the permeation enhancer is transdermal. In some aspects, the permeation enhancer is to facilitate crossing the brain-blood barrier. In some aspects, the permeation enhancer improves the permeability in the oral, nasal, buccal, pulmonary, vaginal, or corneal delivery model. In some aspects, the permeation enhancer is a fatty acid or a derivative thereof. In some aspects, the permeation enhancer is a surfactant or a derivative thereof. In some aspects, the permeation enhancer is a bile salt or a derivative thereof. In some aspects, the permeation enhancer is a chelating agent or a derivative thereof. In some aspects, the permeation enhancer is a non-chelating non-surfactant or a derivative thereof. In some aspects, the permeation enhancer is an ester or a derivative thereof. In some aspects, the permeation enhancer is an ether or a derivative thereof. In some specific aspects, the permeation enhancer is arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof. In one aspect, the permeation enhancer is sodium caprate (C10). In some instances, the permeation enhancer is chenodeoxycholic acid (CDCA), ursodeoxychenodeoxycholic acid (UDCA), cholic acid, dehydrocholic acid, deoxycholic acid, glucholic acid, glycholic acid, glycodeoxycholic acid, taurocholic acid taurodeoxycholic acid, sodium tauro-24,25-dihydro-fusidate or sodium glycodihydrofusidate. In some instances, the permeation enhancer is polyoxyethylene-9-lauryl ether, or polyoxyethylene-20-cetyl ether.

Further provided herein are kits comprising the modulator disclosed herein. Further provided herein are kits comprising the pharmaceutical composition disclosed herein. In some aspects, the kit comprises suitable instructions in order to perform the methods of the kit. The instructions may provide information of performing any of the methods disclosed herein, whether or not the methods may be performed using only the reagents provided in the kit.

For use in the therapeutic applications described herein, kits and articles of manufacture are also described herein. In some aspects, such kits include a carrier, package, or container that is compartmentalized to receive one or more containers such as vials, tubes, and the like, each of the container(s) including one of the separate elements to be used in a method described herein.

Suitable containers include, for example, bottles, vials, syringes, and test tubes. The containers can be formed from a variety of materials such as glass or plastic. The articles of manufacture provided herein contain packaging materials. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. The container(s) optionally have a sterile access port (for example the container is an intravenous solution bag or a vial having a stopper pierceable by a hypodermic injection needle). Such kits optionally comprise a composition with an identifying description or label or instructions relating to its use in the methods described herein.

A kit may include one or more additional containers, each with one or more of various materials (such as reagents, optionally in concentrated form, and/or devices) desirable from a commercial and user standpoint for use of the modulator described herein. Non-limiting examples of such materials include, but not limited to, buffers, diluents, filters, needles, syringes, carrier, package, container, vial and/or tube labels listing contents and/or instructions for use, and package inserts with instructions for use. A set of instructions will also typically be included.

In some aspects, a label is on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain aspects, a pharmaceutical composition comprising the modulators provided herein and optional additional active agent is presented in a pack or dispenser device which can contain one or more unit dosage forms. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert.

Compositions containing the modulators described herein formulated in a compatible pharmaceutical carrier can also be prepared, placed in an appropriate container, and labeled for treatment of an indicated condition.

### Methods of Use

Provided herein comprises methods of modulating the expression or activity of long noncoding transcript disclosed herein by contacting a cell comprising the long noncoding transcript to the modulator disclosed herein, or a pharmaceutical composition disclosed herein. In some instances, the cell is affected by ARDS. In some instances, the ARDS is associated with or resulted from COVID-19 infection. Also provided herein comprises methods of modulating the expression or activity of long noncoding transcript disclosed herein in a subject in need thereof by administering to the subject the modulator disclosed herein, or a pharmaceutical composition disclosed herein. In some instances, the subject has been developing ARDS, affected by ARDS, suffering from one or more symptoms of ARDS, and/or has been infected by viruses (e.g., COVID-19) inducing or causing the onset or development ARDS.

Specifically provided herein are methods of reducing expression or activity of the long noncoding transcript disclosed herein in a subject in need thereof by administering to the subject the modulator disclosed herein, or a pharmaceutical composition disclosed herein. In some instances, the subject has been developing ARDS, affected by ARDS, suffering from one or more symptoms of ARDS, and/or has been infected by viruses (e.g., COVID-19) inducing or causing the onset or development ARDS.

Further provided herein are methods of preventing, alleviating, or treating pulmonary fibrosis or a symptom associated with ARDS in a subject in need thereof, the method comprising administering to the subject an effective amount of the modulator disclosed herein or the pharmaceutical composition provided herein. In some instances, ARDS is associated, induced, caused, or resulted from COVID-19 infection.

Further provided herein are methods of preventing, alleviating, or treating pulmonary fibrosis or a symptom associated with pulmonary fibrosis in a subject in need thereof, the method comprising administering to the subject an effective amount of the modulator disclosed herein or the pharmaceutical composition provided herein. In some instances, the pulmonary fibrosis is associated with ARDS. In some instances, the pulmonary fibrosis is idiopathic pulmonary fibrosis. In some instances, the pulmonary fibrosis is associated with or induced by ARDS that is associated, induced, caused, or resulted from COVID-19 infection.

Further provided herein are methods of preventing, alleviating, or treating inflammation in a subject in need thereof, the method comprising administering to the subject an effective amount of the modulator disclosed herein or the pharmaceutical composition provided herein. In some instances, the inflammation is associated with onset or development of pulmonary fibrosis.

In some instances, the inflammation is associated with onset or development of idiopathic pulmonary fibrosis. In some instances, the inflammation is associated with or induced by onset of ARDS that is associated or resulted from COVID-19 infection.

Further provided herein are methods of preventing, alleviating, or treating myocardial fibrosis, pancreatic fibrosis, adipose tissue fibrosis, intestinal fibrosis, and/or uterine fibrosis or a symptom associated with such fibrosis in a subject in need thereof, the method comprising administering to the subject an effective amount of the modulator disclosed herein of a lncRNA transcript derived from the human genomic location LOC107986083 (chr3: 45,817,379-45,827,511) or the pharmaceutical composition provided herein that modulates a lncRNA transcript derived from the human genomic location LOC107986083 (chr3: 45,817,379-45,827,511). Genotype-Tissue Expression (GTEx) analysis indicated that CORAL transcripts containing sequence derived from the human genomic location LOC107986083 (chr3: 45,817,379-45,827,511) is expressed at a moderate level in some tissues of mesenchymal origin such as lung and instestine. Expression of these hsCORAL transcripts was also detected in pancreas. Detectable, but low levels of expression of these hsCORAL transcripts was found in muscle, uterus, and skin. Expression of these hsCORAL transcripts was very low-to-undetectable in other tissues assayed.

In some aspects, the subject in need of treatment has or is suspected to have ARDS. In some instances, the subject has or is suspected to have ARDS that is caused by or associated with viral infection. In some specific aspects, the subject has or is suspected to have ARDS that is caused by or associated with viral pulmonary disease. In further specific aspects, the subject has or is suspected to have ARDS that is caused by or associated with COVID-19 infection. In some instances, the subject has or is suspected to have ARDS that is caused by or associated with HIV. In some instances, the subject has or is suspected to have ARDS that is caused by or associated with an increased immune response. In some instances, the subject has or is suspected to have ARDS that is caused by or associated with an autoimmune disease.

In some aspects, the effective amount of a modulator as disclosed herein can decrease the amount, expression level, or activity of the long noncoding transcript disclosed herein at least 1%, 2%, 3%, 4%, 5%, 10%, 20%, 30%, 40%, 50%, 60%, 70%, 80%, 90% or more in the cell contacted with the modulator. In some aspects, the effective amount of a modulator as disclosed herein can decrease the amount, expression level, or activity of the long noncoding transcript disclosed herein at least 1%, 2%, 3%, 4%, 5%, 10%, 20%, 30%, 40%, 50%, 60%, 70%, 80%, 90% or more in the cells or tissues of the subject affected by pulmonary fibrosis, e.g., pulmonary fibrosis associated with ARDS, affected by ARDS, and/or affected by COVID-19 infection.

In some aspects, the effective amount of a modulator as disclosed herein can modulate the amount, expression level, or activity of transcripts or mRNAs of genes associated with or markers of pulmonary fibrosis, e.g., pulmonary fibrosis associated with or induced by ARDS in a at least 1%, 2%, 3%, 4%, 5%, 10%, 20%, 30%, 40%, 50%, 60%, 70%, 80%, 90% or more in the cells or tissues of the subject affected by pulmonary fibrosis, e.g., pulmonary fibrosis associated with ARDS, affected by ARDS, and/or affected by COVID-19 infection. In some aspects, the effective amount of a modulator as disclosed herein can increase the amount, expression level, or activity of transcripts or mRNAs of at least one or more genes associated with or markers of pulmonary fibrosis, e.g., pulmonary fibrosis associated with or induced by ARDS in a at least 1%, 2%, 3%, 4%, 5%, 10%, 20%, 30%, 40%, 50%, 60%, 70%, 80%, 90% or more in the cells or tissues of the subject affected by pulmonary fibrosis, e.g., pulmonary fibrosis associated with ARDS, affected by ARDS, and/or affected by COVID-19 infection. In some aspects, the effective amount of a modulator as disclosed herein can decrease the amount, expression level, or activity of transcripts or mRNAs of at least one or more genes associated with or markers of pulmonary fibrosis, e.g., pulmonary fibrosis associated with or induced by ARDS in a at least 1%, 2%, 3%, 4%, 5%, 10%, 20%, 30%, 40%, 50%, 60%, 70%, 80%, 90% or more in the cells or tissues of the subject affected by pulmonary fibrosis, e.g., pulmonary fibrosis associated with ARDS, affected by ARDS, and/or affected by COVID-19 infection.

In some aspects, the effective amount of a modulator as disclosed herein can alleviate or reduce the severity or frequencies of symptoms of pulmonary fibrosis associated with ARDS or severity or frequencies of symptoms of ARDS. In some aspects, the effective amount of a modulator as disclosed herein can reverse severity or frequencies symptoms of pulmonary fibrosis associated with ARDS or severity or symptoms of ARDS, or progress of pulmonary fibrosis associated with ARDS or ARDS.

In some aspects, the modulator is expressed in a viral vector. In other aspects, the modulator is expressed in a plasmid vector. In some instances, the modulator is encapsulated in a liposome. In some instances, the modulator is encapsulated in a nanoparticle. In some instances, the modulator is encapsulated in an extracellular vesicle.

For delivery to the target cell, the modulator described herein can non-covalently bind an excipient to form a complex. The excipient can be used to alter biodistribution after delivery, to enhance uptake, to increase half-life or stability of the strands in the modulator described herein (e.g., improve nuclease resistance), and/or to increase targeting to a particular cell or tissue type.

Exemplary excipients include but are not limited to a condensing agent (e.g., an agent capable of attracting or binding a nucleic acid through ionic or electrostatic interactions); a fusogenic agent (e.g., an agent capable of fusing and/or being transported through a cell membrane); a protein to target a particular cell or tissue type (e.g., thyrotropin, melanotropin, lectin, glycoprotein, surfactant protein A, or any other protein); a lipid; a lipopolysaccharide; a lipid micelle or a liposome (e.g., formed from phospholipids, such as phosphotidylcholine, fatty acids, glycolipids, ceramides, glycerides, cholesterols, or any combination thereof); a nanoparticle (e.g., silica, lipid, carbohydrate, or other pharmaceutically-acceptable polymer nanoparticle); a polyplex formed from cationic polymers and an anionic agent (e.g., a CRO), where exemplary cationic polymers include but are not limited to polyamines (e.g., polylysine, polyarginine, poly amidoamine, and polyethylene imine); cholesterol; a dendrimer (e.g., a polyamidoamine (PAMAM) dendrimer); a serum protein (e.g., human serum albumin (HSA) or low-density lipoprotein (LDL)); a carbohydrate (e.g., dextran, pullulan, chitin, chitosan, inulin, cyclodextrin, or hyaluronic acid); a lipid; a synthetic polymer, (e.g., polylysine (PLL), polyethylenimine, poly-L-aspartic acid, poly-L-glutamic acid, styrene-maleic acid anhydride copolymer, poly(L-lactide-co-glycolic) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacrylic acid), N-isopropylacrylamide polymer, pseudopeptide-polyamine, peptidomimetic polyamine, or polyamine); a cationic moiety (e.g., cationic lipid, cationic porphyrin, quaternary salt of a polyamine, or alpha helical peptide); a multivalent sugar (e.g., multivalent lactose, multivalent galactose, N-acetyl-galactosamine, N-acetyl-glucosamine, multivalent mannose, or multivalent fucose); a vitamin (e.g., vitamin A, vitamin E, vitamin K, vitamin B, folic acid, vitamin B12, riboflavin, biotin, or pyridoxal); a cofactor; or a drug to disrupt cellular cytoskeleton to increase uptake (e.g., taxol, vincristine, vinblastine, cytochalasin, nocodazole, japlakinolide, latrunculin A, phalloidin, swinholide A, indanocine, or myoservin).

In some aspects, the administering is performed intratracheally, orally, nasally, intravenously, intraperitoneally, or intramuscularly. In some aspects, the administering is performed intratracheally.

In some aspects, the administering is a targeted delivery to a lung tissue of the subject. In some instances, the targeted delivery is via a local application. In some instances, the targeted delivery is via one or more specific binding moieties that target the lung tissue.

In some aspects, the administering is in a form of aerosol. In some instances, the aerodynamic diameter of particles of the modulator disclosed herein is less than 10 μm. In some instances, the aerodynamic diameter of particles of the modulator disclosed herein is less than 5 μm. In some instances, the aerodynamic diameter of particles of the modulator disclosed herein is less than 3 μm.

Further provided herein are methods of diagnosing or monitoring pulmonary fibrosis in a subject, the method comprising: (a) obtaining a sample from the subject; (b) detecting an amount and/or an activity of a long noncoding transcript, wherein the long noncoding transcript is transcribed from a genomic region LOC107986083; and (c) diagnosing the subject with pulmonary fibrosis or to have a high/higher chance to contract pulmonary fibrosis if the amount and/or the activity is at least 50%, at least 60%, at least 70%, at least 80%, at least 90% higher when compared to a control. In some aspects, the detecting comprises using Si nuclease protection assay, microarray analysis, polymerase chain reaction (PCR), hybridization technologies, reverse transcriptase polymerase chain reaction (RT-PCR), Northern blot, serial analysis of gene expression (SAGE), immunoassay, and/or mass spectrometry.

Further provided herein are methods of diagnosing or monitoring idiopathic pulmonary fibrosis in a subject, the method comprising: (a) obtaining a sample from the subject; (b) detecting an amount and/or an activity of a long noncoding transcript, wherein the long noncoding transcript is transcribed from a genomic region LOC107986083; and (c) diagnosing the subject with idiopathic pulmonary fibrosis or to have a high/higher chance to contract idiopathic pulmonary fibrosis if the amount and/or the activity is at least 50%, at least 60%, at least 70%, at least 80%, at least 90% higher when compared to a control. In some aspects, the detecting comprises using S1 nuclease protection assay, microarray analysis, polymerase chain reaction (PCR), hybridization technologies, reverse transcriptase polymerase chain reaction (RT-PCR), Northern blot, serial analysis of gene expression (SAGE), immunoassay, and/or mass spectrometry.

In some aspects, the method of treating pulmonary fibrosis in a subject comprises: (a) obtaining a sample from the subject; (b) detecting an amount and/or an activity of a long noncoding transcript, wherein the long noncoding transcript is transcribed from a genomic region LOC107986083; (c) diagnosing the subject with pulmonary fibrosis if the amount and/or the activity is at least 50%, at least 60%, at least 70%, at least 80%, at least 90% higher when compared to a control; and (d) treating the subject with an effective amount of the modulators disclosed herein. In some aspects, the detecting comprises using Si nuclease protection assay, microarray analysis, polymerase chain reaction (PCR), hybridization technologies, reverse transcriptase polymerase chain reaction (RT-PCR), Northern blot, serial analysis of gene expression (SAGE), immunoassay, and/or mass spectrometry.

In some aspects, the method of treating idiopathic pulmonary fibrosis in a subject comprises: (a) obtaining a sample from the subject; (b) detecting an amount and/or an activity of a long noncoding transcript, wherein the long noncoding transcript is transcribed from a genomic region LOC107986083; (c) diagnosing the subject with idiopathic pulmonary fibrosis if the amount and/or the activity is at least 50%, at least 60%, at least 70%, at least 80%, at least 90% higher when compared to a control; and (d) treating the subject with an effective amount of the modulators disclosed herein. In some aspects, the detecting comprises using Si nuclease protection assay, microarray analysis, polymerase chain reaction (PCR), hybridization technologies, reverse transcriptase polymerase chain reaction (RT-PCR), Northern blot, serial analysis of gene expression (SAGE), immunoassay, and/or mass spectrometry.

Further provided herein are methods of monitoring or predicting the clinical development of a SARS-CoV2 infection in a subject having or suspected of having the infection, the method comprising: (a) obtaining a sample from the subject; (b) detecting an amount and/or an activity of a long noncoding transcript, wherein the long noncoding transcript is transcribed from a genomic region LOC107986083; and (c) diagnosing the subject with the infection or determining the subject having a high/higher chance to develop clinically substantive symptoms from the SARS-CoV2 infection if the amount and/or the activity is at least 50%, at least 60%, at least 70%, at least 80%, at least 90% higher when compared to a control. In some aspects, the detecting comprises using S1 nuclease protection assay, microarray analysis, polymerase chain reaction (PCR), hybridization technologies, reverse transcriptase polymerase chain reaction (RT-PCR), Northern blot, serial analysis of gene expression (SAGE), immunoassay, and/or mass spectrometry.

In some aspects, methods of managing a SARS-CoV2 infection in a subject having or suspected of having the infection comprises (a) obtaining a sample from the subject; (b) detecting an amount and/or an activity of a long noncoding transcript, wherein the long noncoding transcript is transcribed from a genomic region LOC107986083; (c) diagnosing the subject with the infection or determining the subject having a high/higher chance to develop clinically substantive symptoms from the SARS-CoV2 infection if the amount and/or the activity is at least 50%, at least 60%, at least 70%, at least 80%, at least 90% higher when compared to a control; and (d) treating the subject with an advanced procedure and/or therapeutics preemptively. In some instances, step (d) comprises use of albumin for resuscitation. In some instances, step (d) comprises use of norepinephrine as a vasopressor. In some specific aspects, step (d) comprises titrating vasoactive agents to target a mean arterial pressure (MAP) of 60 to 65 mm Hg. In some instances, step (d) comprises adding either vasopressin (up to 0.03 units/min) or epinephrine to norepinephrine to raise MAP. In some instances, step (d) comprises adding vasopressin (up to 0.03 units/min) to decrease norepinephrine dosage. In some instances, step (d) comprises a low-dose dopamine for renal protection. In some instances, step (d) comprises dobutamine. In some instances, step (d) comprises corticosteroids. In some instances, step (d) comprises high-flow nasal cannula (HFNC) oxygen. In some instances, step (d) comprises a closely monitored trial of noninvasive ventilation. In some instances, step (d) comprises a trial of awake prone positioning.

In some instances, step (d) comprises intubation. In some instances, step (d) comprises low tidal volume (VT) ventilation (VT 4-8 mL/kg of predicted body weight). In some instances, step (d) comprises using a higher positive end-expiratory pressure. In some instances, step (d) comprises prone ventilation for 12 to 16 hours per day. In some instances, step (d) comprises using intermittent boluses of neuromuscular blocking agents. In some instances, step (d) comprises recruitment maneuvers. In some instances, step (d) comprises using an inhaled pulmonary vasodilator as a rescue therapy. In some instances, step (d) comprises a continuous renal replacement therapy. In some instances, step (d) comprises a use of empiric broad-spectrum antimicrobial therapy. In some instances, step (d) comprises a use of extracorporeal membrane oxygenation.

In some aspects, the clinical development comprises acute respiratory distress syndrome (ARDS). In other aspects, the clinical development comprises asthma, chronic obstructive pulmonary diseases (COPD), and/or Chronic mucus hypersecretion (CMH) pathogenesis. In some aspects, the clinical development comprises idiopathic pulmonary fibrosis (IPF).

In some aspects, the detecting comprises using Si nuclease protection assay, microarray analysis, polymerase chain reaction (PCR), hybridization technologies, reverse transcriptase polymerase chain reaction (RT-PCR), Northern blot, serial analysis of gene expression (SAGE), immunoassay, and/or mass spectrometry.

### Certain Terminology

The term “noncoding RNA” as used herein, refers to RNA species that are not translated into protein.

The term “CORAL” as used herein, refers to a long noncoding transcript that is associated with onset, development, or prognosis of ARDS and/or pulmonary fibrosis associated with, induced by, or resulted from ARDS. CORAL includes, but not limited to, a long noncoding transcript that is associated with onset, development, or prognosis of ARDS developed after, directly or indirectly, COVID-19 infection, and/or pulmonary fibrosis associated with, induced by, or resulted from COVID-19 infection. As such, CORAL includes, but not limited to, a long noncoding transcript transcribed from a genomic region LOC107986083 as described herein.

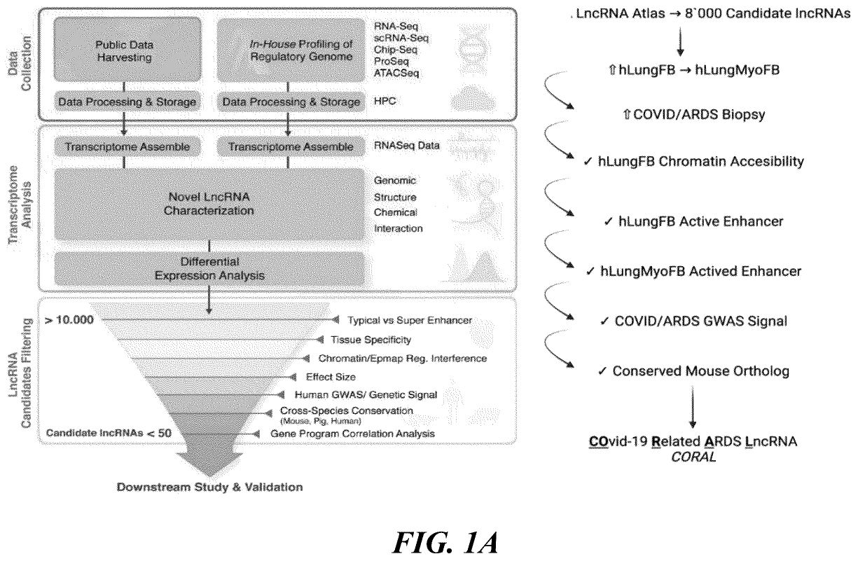

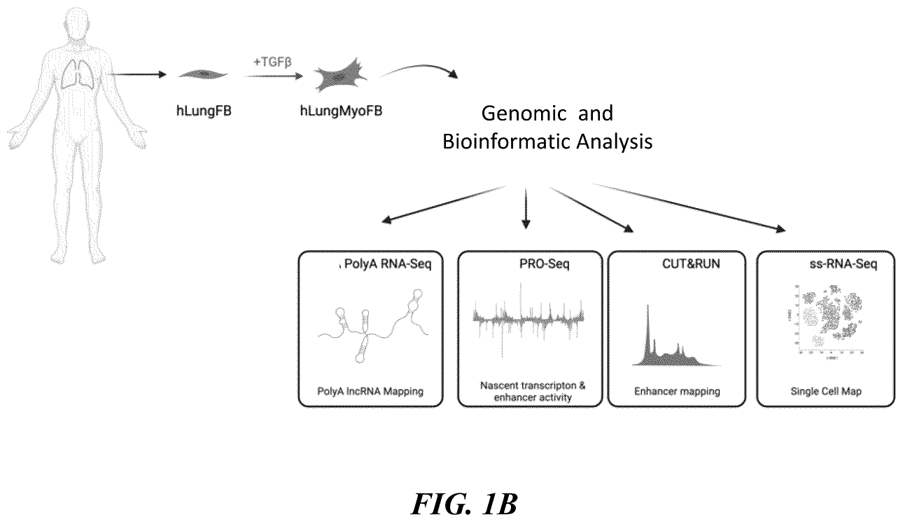

CORAL includes, but not limited to, a long noncoding transcript transcribed from a genomic region LOC126806670. CORAL includes, but not limited to, a long noncoding transcript transcribed from a genomic region that is identified by using the methods described in the flowchart listed in FIG. 1A.

The term “baseline level” of a transcript as used herein, refers to the level of the transcript in healthy cells of the subject or the cells of the healthy subject. In some aspects, it refers to the level of the long noncoding transcripts in healthy cells of a healthy individual. In some aspects, it refers to the level of the long noncoding transcripts in cells of the same subject but before ARDS.

The term “nucleic acid editing or modifying moiety,” as used herein, refers to a moiety that edits or cleaves the target nucleic acid. It can also refer to a moiety that suppresses the transcription of the target nucleic acid.

The term “SARS-CoV2” and “COVID 19” are used sometimes interchangeably, either refer to the virus or the infection caused by the virus.

The term “nucleic acid analogue,” as used herein, refers to compounds which are analogous (structurally similar) to naturally occurring nucleic acid (see, e.g., Freier & Altmann; Nucl. Acid. Res., 1997, 25, 4429-4443 and Uhlmann; Curr. Opinion in Drug Development, 2000, 3(2), 293-213), and examples of suitable nucleic acid analogues are provided by WO2007031091, which are hereby incorporated by reference.

The term “gapmer” is a chimeric nucleic acid that contains a central sequence of DNA nucleotides (“DNA gap”) flanked by sequences of modified RNA residues at either end to protect the DNA gap from nuclease degradation, whereas the central DNA gap region allows RNase-H-mediated cleavage of the target RNA. Gapmer has an internal region having a plurality of nucleosides which is capable of recruiting RNase H activity, such as RNaseH, which region is positioned between external wings at either end, having one or more nucleosides, wherein the nucleosides comprising the internal region are chemically distinct from the nucleoside or nucleosides comprising the external wings.

A “locked nucleic acid” or “LNA” is often referred to as inaccessible RNA, and is a modified RNA nucleobase. The ribose moiety of an LNA nucleobase is modified with an extra bridge connecting the 2′ oxygen and 4′ carbon. An LNA oligonucleotide offers substantially increased affinity for its complementary strand, compared to traditional DNA or RNA oligonucleotides.

The terms “microRNA,” “miRNA,” and MiR” are interchangeable and refer to endogenous or artificial non-coding RNAs that are capable of regulating gene expression. It is believed that miRNAs function via RNA interference. The terms “siRNA” and “short interfering RNA” are interchangeable and refer to single-stranded or double-stranded RNA molecules that are capable of inducing RNA interference. In some aspects, siRNA molecules typically have a duplex region that is between 18 and 30 base pairs in length.

The terms “piRNA” and “Piwi-interacting RNA” are interchangeable and refer to a class of small RNAs involved in gene silencing. piRNA molecules typically are between 26 and 31 nucleotides in length.

The terms “snRNA” and “small nuclear RNA” are interchangeable and refer to a class of small RNAs involved in a variety of processes including RNA splicing and regulation of transcription factors. The subclass of small nucleolar RNAs (snoRNAs) is also included. The term is also intended to include artificial snRNAs, such as antisense derivatives of snRNAs comprising antisense sequences directed against one or more lncRNAs.

The terms “polynucleotide” “oligonucleotide” “polynucleic acid”, “nucleic acid”, and “nucleic acid molecule” are used herein to include a polymeric form of nucleotides of any length, either ribonucleotides, deoxyribonucleotides, modified forms thereof, or hybrid or chimeric forms thereof. This term refers to the primary structure of the molecule. Thus, the term includes triple-, double- and single-stranded DNA, as well as triple-, double- and single-stranded RNA. In some aspects, it also includes modifications, such as by methylation and/or by capping, and unmodified forms of the polynucleotide. More particularly, the terms “polynucleotide” “oligonucleotide” “polynucleic acid”, “nucleic acid”, and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing non-nucleotidic backbones, for example, polyamide (e.g., peptide nucleic acids (PNAs)) and polymorpholino (commercially available from the Anti-Vials, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide” “oligonucleotide” “polynucleic acid”, “nucleic acid”, and “nucleic acid molecule” and these terms will be used interchangeably. Thus, these terms include, for example, RNA, double- and single-stranded DNA, as well as double- and single-stranded RNA, microRNA, DNA:RNA hybrids, and hybrids between PNAs and DNA or RNA, and also include known types of modifications, for example, labels which are known in the art, methylation, “caps,” substitution of one or more of the naturally occurring nucleotides with an analog (e.g., 2-aminoadenosine, 2′-thiothymidine, inosine, pyrrolo-pyrimidine, 3′-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, 0(6)-methylguanine, and 2′-thiocytidine), internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. The term also includes locked nucleic acids (e.g., comprising a ribonucleotide that has a methylene bridge between the 2′-oxygen atom and the 4′-carbon atom). See, for example, Kurreck et al. (2002) Nucleic Acids Res. 30: 1911-1918.

The term “complementary” and “complementarity” are interchangeable and refer to the ability of polynucleotides to form base pairs with one another. Base pairs are typically formed by hydrogen bonds between nucleotide units in antiparallel polynucleotide strands or regions.

Complementary polynucleotide strands or regions can base pair in the Watson-Crick manner (e.g., A to T, A to U, C to G). 100% complementary refers to the situation in which each nucleotide unit of one polynucleotide strand or region can hydrogen bond with each nucleotide unit of a second polynucleotide strand or region. Less than perfect complementarity refers to the situation in which some, but not all, nucleotide units of two strands or two regions can hydrogen bond with each other and can be expressed as a percentage.

The term “administering”, as it applies in the present disclosure, refers to contact of an effective amount of a modulator of one or more lncRNAs of the disclosure, to the subject.

Administering a nucleic acid, such as a microRNA, siRNA, piRNA, snRNA, or antisense nucleic acid, to a cell comprises transducing, transfecting, electroporating, translocating, fusing, phagocytosing, shooting or ballistic methods, or any means by which a nucleic acid can be transported across a cell membrane.

The term “pharmaceutically acceptable excipient or carrier” refers to an excipient that may optionally be included in the compositions of the disclosure and that causes no significant adverse toxicological effects to the patient.

The term “pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

An “effective amount” of modulator of one or more lncRNAs of the disclosure (e.g., microRNA, siRNA, piRNA, snRNA, antisense nucleic acid, ribozyme, or small molecule inhibitor, CRISPRs etc.) is an amount sufficient to effect any beneficial or desired results, such as an amount that inhibits the activity of a lncRNA, at any level, for example by interfering with transcription. An effective amount can be administered in one or more administrations, applications, or dosages.

By “therapeutically effective dose or amount” of a modulator of one or more lncRNAs of the disclosure is intended an amount that, when administered as described herein, brings about a positive therapeutic response. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

“Homology” refers to the percent identity between two polynucleotides or two polypeptide moieties. Two nucleic acid sequences, or two polypeptide sequences are “substantially homologous” to each other when the sequences exhibit at least about 50% sequence identity, at least about 75% sequence identity, at least about 80%-85% sequence identity, at least about 90% sequence identity, or about 95%-98% sequence identity over a defined length of the molecules. As used herein, substantially homologous sequences also refer to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Alternatively, homology can be determined by readily available computer programs or by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art.

As used herein, a “sample” refers to a sample of tissue or fluid isolated or obtained from a subject, including but not limited to, for example, urine, blood, plasma, serum, fecal matter, bone marrow, bile, spinal fluid, lymph fluid, samples of the skin, external secretions of the skin, respiratory, intestinal, and genitourinary tracts, tears, saliva, milk, blood cells, organs, biopsies, and also samples containing cells or tissues derived from the subject and grown in culture, and in vitro cell culture constituents, including but not limited to, conditioned media resulting from the growth of cells and tissues in culture, recombinant cells, stem cells, and cell components.

The terms “quantity,” “amount,” and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

“Diagnosis” as used herein generally includes determination as to whether a subject is likely affected by a given disease, disorder or dysfunction of the disclosure. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

“IMR-90” refers to a commonly used immortalized human lung fibroblast cell line available at ATCC.

Whenever the term “at least,” “more than,” or “less than” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

The term “a,” “an” and “the” include plural referents unless the content clearly dictates otherwise. Thus, for example, reference to “a lncRNA” includes a mixture of two or more lncRNAs, and the like.

The term “about,” particularly in reference to a given quantity, is meant to encompass deviations of plus or minus ten percent.

The term “pulmonary fibrosis” as used herein, refers to a set of lung diseases that affect the respiratory system. In some aspects, pulmonary fibrosis refers to thickening or scarring of the lung tissue or a portion thereof. In some aspects, pulmonary fibrosis is idiopathic pulmonary fibrosis.

### Examples

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. Efforts have been made to ensure accuracy with respect to numbers used (e.g., amounts, percentages, etc.), but some experimental errors and deviations should be accounted for.

### Example 1: Genetic profiling—of LOC107986083

Genetic characterization of LOC107986083 was carried out on different levels as depicted in the flow chart of FIG. 1A.

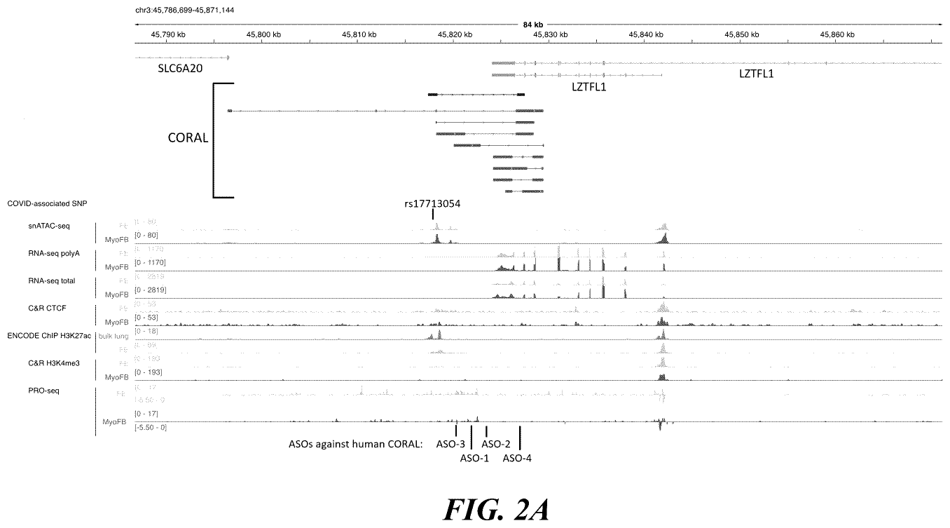

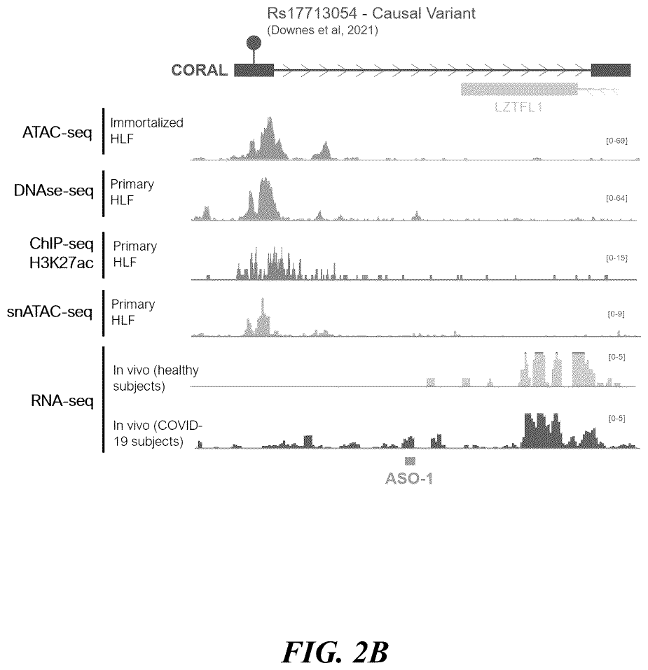

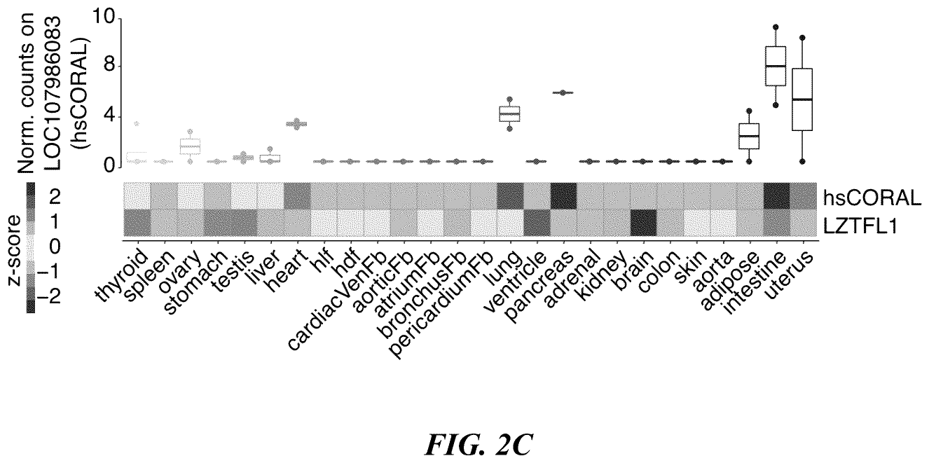

First, to gain an understanding of chromosomal regulation of LOC107986083, ATAC-seq, DNAse-seq, and ChIP-seq with H3K27ac were conducted and analyzed. ATAC-seq was performed on cells from an immortalized human lung fibroblast (HLF) cell line. “HLF” stands for normal primary human lung fibroblasts isolated from healthy donors. DNase-Seq was performed on primary HLF cells. ChIP-Seq H3K27ac was performed on primary HLF cells. snATAC-Seq was performed on primary HLF cells. RNA-Seq was performed on cells from in vivo biopsies taken from healthy individuals, or subjects exhibiting one or more symptoms of COVID-19, or subjects having exhibited one or more symptoms of COVID-19. Briefly, processed bam files for the conditions shown in FIG. 2A were obtained from the ENCODE project, and were loaded into Integrative Genomics Viewer (IGV) for visualization. LOC107986083 presents DNAse-accessible and ATAC-seq accessible sites, and H3K27ac enhancer marker was found to be located in the first exon of an annotation of the LOC107986083 transcript. These results indicate that an enhancer region annotated in FIG. 2A is active in vitro. Furthermore, nascent transcription of LOC107986083 was investigated with PRO-seq. PRO-seq was performed with standardized methods in the field (see, e.g., Mahat et al., Nat Protoc. 2016 Aug; 11(8): 1455-1476). Sequencing was performed as SR75 on an Illumina HiSeq 4000 sequencing system. FASTQ files were cleaned using Trimmomatic 13. Subsequently, the trimmed FASTQ files were aligned to the hg38 reference genome using Bowtie2. The resulting bam files were loaded into IGV for visualization. PRO-seq results are shown in FIG. 2A. Under control conditions, nascent transcription was observed to be paused, while cells undergoing serum starvation and being treated with TGFβ underwent active transcription. Next, deep RNA-Seq was carried out to investigate transcription products of LOC107986083. Specifically, RNA was isolated with the RNeasy RNA extraction kit from Qiagen (ref. 74106), and was reverse transcribed using the Quantitect reverse transcription kit from Qiagen (ref. 205311). Deep RNA-Seq was performed on a NovaSeq 6000 PE150 with ribodepleted Illumina TruSeq® library preparation. Samples were sequenced at a depth of up to 400M reads/sample. Raw sequencing data (FASTQ files) were mapped to the Gencode hg38 reference genome using STAR, with the following parameters: —outFilterMultimapNmax 25—outFilterIntronMotifs RemoveNoncanonical. Gene expression was quantified using the tool featureCounts from the software Subread. Quantification was performed on different reference transcriptomes, notably including Gencode and an in-house human lung fibroblast reconstructed transcriptome. The in-house reconstructed transcriptome was generated from previous experiments, using a transcript assembly pipeline based on StringTie and Cuffmerge tools. hsCORAL isoforms annotated in FIG. 2A were sequenced with capture followed by PacBio IsoSeq. A panel of dsDNA probes was designed targeting the region of interest on human chr3. PacBio Iso-Seq (polyA) was performed after a capture of the RNA of interest and polyadenylated hsCORAL isoforms were reconstructed.

### Probes Design

The chr3:45796893-45863928 (hg38) region was tiled with custom 1× dsDNA probes, subtracting all known exons (GENCODE) from protein-coding gene LZTFL1. Probes were synthetized with Twist Bioscience (ref. 101001).

### Capture, Library, and Sequencing

High Quality Total RNA (RIN>9) was extracted from Human Lung Fibroblasts (FB and MyoFB conditions) using High Pure RNA Isolation Kit (Roche, Cat. N°11828665001) according to the manufacturer protocol. Libraries, enrichment, and sequencing were performed by Lausanne Genomic Technologies Facility (GTF). Briefly, PacBio Isoform Sequence (Iso-Seq) libraries were prepared per manufacturer's instructions. A target enrichment step using Twist reagents, probes panel and protocol was done between the cDNA amplification and End repair steps of the Iso-seq library. A change was applied to target enrichment Twist protocol: Universal Blockers were replaced by custom PacBio compatible blocker. Libraries were sequenced on a Sequel II instrument.

### Analysis Method

Samples were processed following the standard PacBio isoseq3 (1) workflow: PacBio Circular Consensus Sequencing (CCS) reads were demultiplexed and refined (isoseq3 refine) to remove polyA tails and artificial concatemers. Full-length non-concatemer (flnc) reads from replicate runs of the same condition were combined into one file-of-filename (fofn). Fofn reads were then clustered (isoseq3 cluster), mapped with pbmm2 (—preset ISOSEQ—sort) (2) to Gencode's human GRCh38 genome, and collapsed (isoseq3 collapse) into unique isoforms. Collapsed isoforms of the target of interest were extracted from each condition sample and merged into one non-redundant set of isoforms using tama-merge (-a 200-z 200) (3). This set was curated to remove mono-exonic as well as isoforms representing less than 2.5% of the captured locus reads, yielding the final robust set of 8 human CORAL isoforms.

In addition, tissue-specific expression of LOC107986083 was studied, and the results are shown in FIG. 2C. Bulk RNA-Seq bam files were retrieved from the ENCODE project website. Counting on LOC107986083 was performed to assess expression of this locus in the corresponding tissues. Gene expression was quantified as described above with featureCounts, using the UCSC RefSeq hg38 transcriptome as reference. Gene expression normalization was performed with the R package DESeq2, producing normalized counts in output.

A comprehensive characterization of the transcription of LOC107986083 and its regulation mechanisms revealed that LOC107986083 is involved in pulmonary fibrosis. Equally important, LOC107986083 exhibited an elevated transcription level in response to fibrosis associated with acute respiratory distress syndrome (ARDS). Furthermore, rs17713054, a causal variant for COVID-19 respiratory failure, was found to be located in the enhancer region of LOC107986083 (see FIG. 2A-FIG. 2B). Isoforms of LOC107986083 and nearby genomic loci were further characterized as summarized in FIG. 2B. Expression of hsCORAL and LZTFL1 in various human tissue types was analyzed and the results are shown in FIG. 2C.

Various isoforms of hsCORAL transcripts comprise rs17713054. Other distinct isoforms of hsCORAL transcripts do not comprise rs17713054.

Further validation and characterization of LOC107986083 are carried out.

LOC107986083 expression in hLung myofibroblasts of different genotypes and changes of its expression in hLung myofibroblasts harboring risk variants are investigated. Specifically, NHLF cell lines from different donors are purchased from Lonza. gDNA is extracted with the GeneJET Genomic DNA Purification Kit (K0721) and Sanger sequencing is performed on the LOC107986083 locus to assess the presence of the single nucleotide polymorphism (SNP) of interest. All cell lines are subsequently treated with serum starvation and TGFβ as described herein, and qPCR or RNA-Seq is performed to evaluate expression levels of the LOC107986083 locus. For HLF datasets or other public data without gDNA information or SNP calling available, a variant calling pipeline based on the software GATK is employed. The RNA-Seq workflow starts from raw sequencing reads that are first mapped to the reference using STAR aligner (basic 2-pass method) to produce a bam file. Duplicates are marked and removed with Picard's MarkDuplicate. Downstream analysis steps involve a base recalibration performed with BQSR and variant calling performed with HaplotypeCaller. The output comprises a VCF file (one per sample) with the list of variants called.

Enhancer activity and CEBPO binding of LOC107986083 in lung myofibroblasts harboring risk variants are studied. PRO-seq is performed as described herein. Changes observed in nascent RNA after analysis is performed to demonstrate changes in enhancer activity at the LOC107986083 locus. Separately, CEBPO and H3K27Ac CUT&RUN and PRO-Seq in genotyped lung fibroblasts-myofibroblasts with or without serum starvation and TGFβ treatment are carried out as shown in FIG. 1B. CUT&RUN method is performed with CEBPO or H3K27Ac, as described herein. Increased CEBPO binding and enhancer activity in myofibroblasts harboring risk variants are tested, and the purchased cell lines are identified as those with the variants.

Furthermore, LOC107986083 expression in GTEX datasets from lung of different genotypes are examined, and its expression is expected to be increased in GTEX samples harboring risk variants. Raw and processed data were obtained from GTEx after obtaining dbGaP access to repository phs000424. Gene expression data used in downstream analyses are preprocessed using the same procedure as described above. Briefly, FASTQ files are mapped to the reference genome, and expression levels of loci of interest across different samples are quantified. Variants are preselected based on genomic regions of interest (e.g., LOC107986083 genomic locus). The expression of lncRNA loci is quantified using the Subread software package, as described herein. The association between expression levels and SNP presence is established. Simple multivariate linear or logistic regression models are employed to perform a statistical analysis since only the association between relatively few SNPs and gene expression is of interest. Multiple covariates can be introduced in the model, e.g., to account for sample features such as tissue of origin or donor sex/ethnicity. Samples are thus stratified by tissue type and presence or absence of SNPs of interest.

### Example 2: Establishing an in-vitro model for pulmonary fibrosis

An in-vitro model was established and was validated to be a faithful model for pulmonary fibrosis.

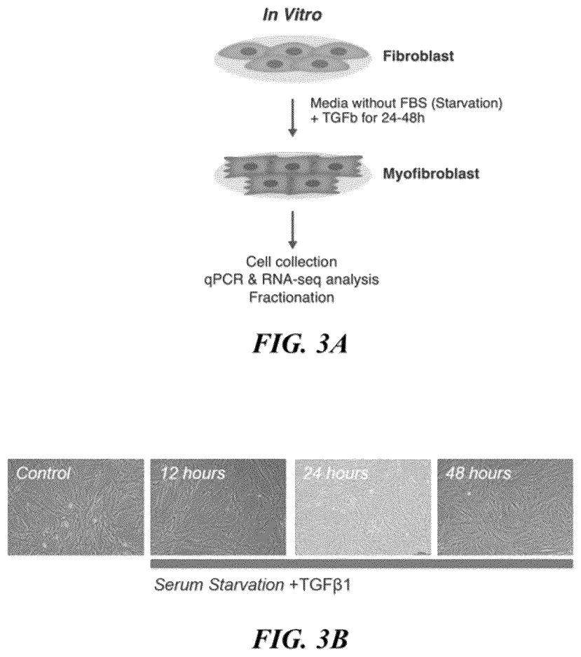

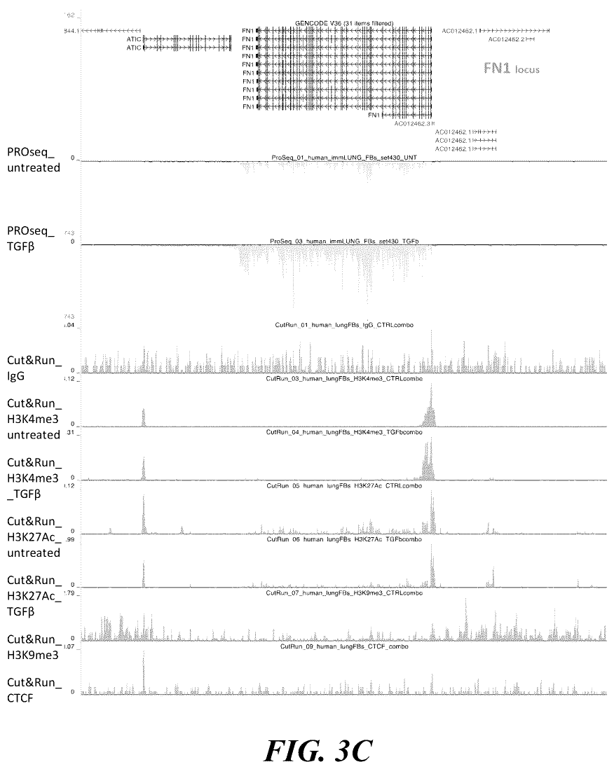

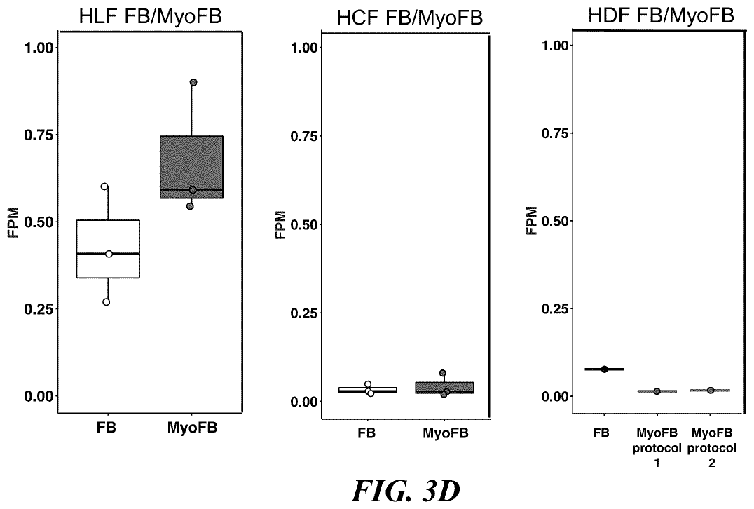

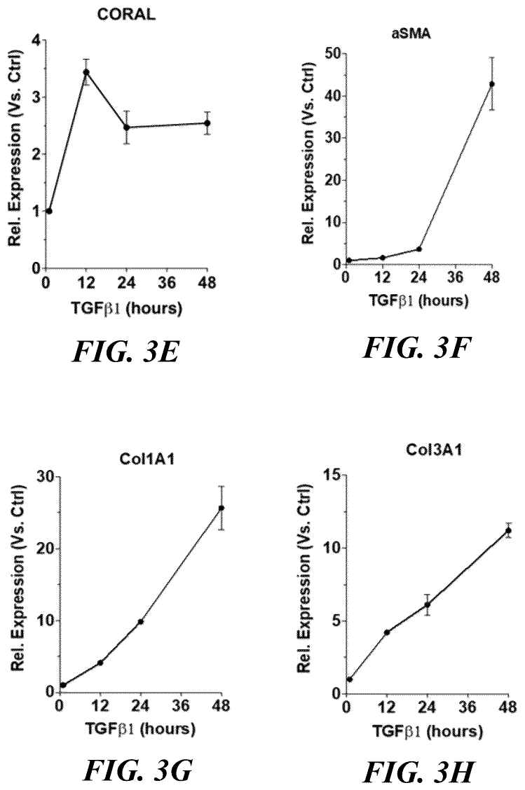

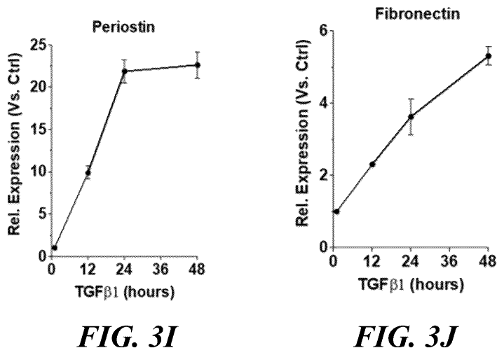

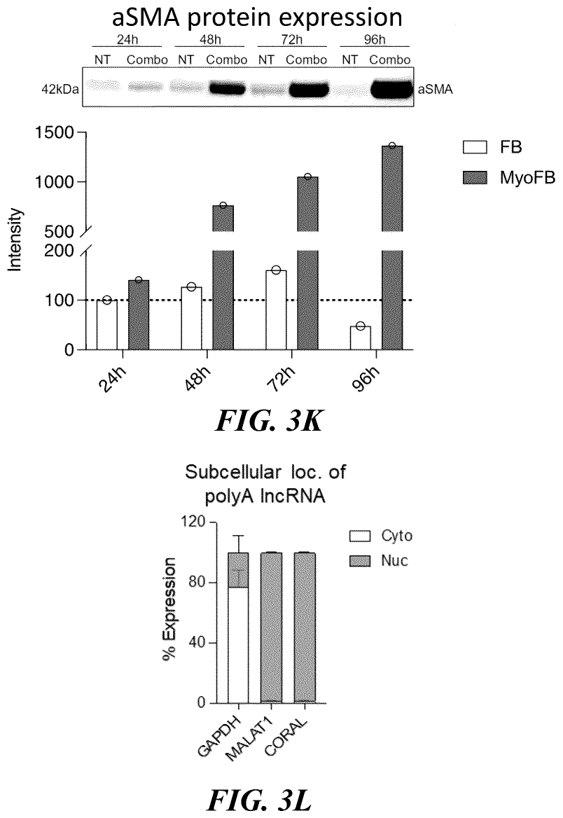

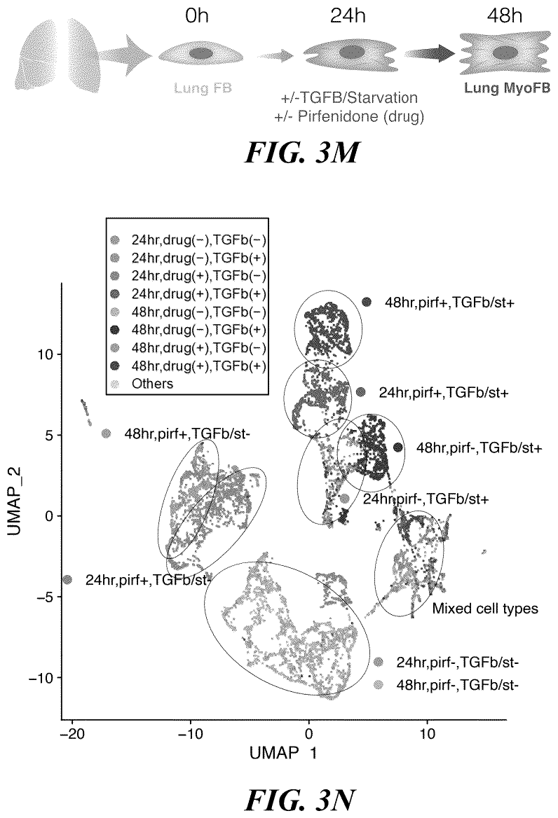

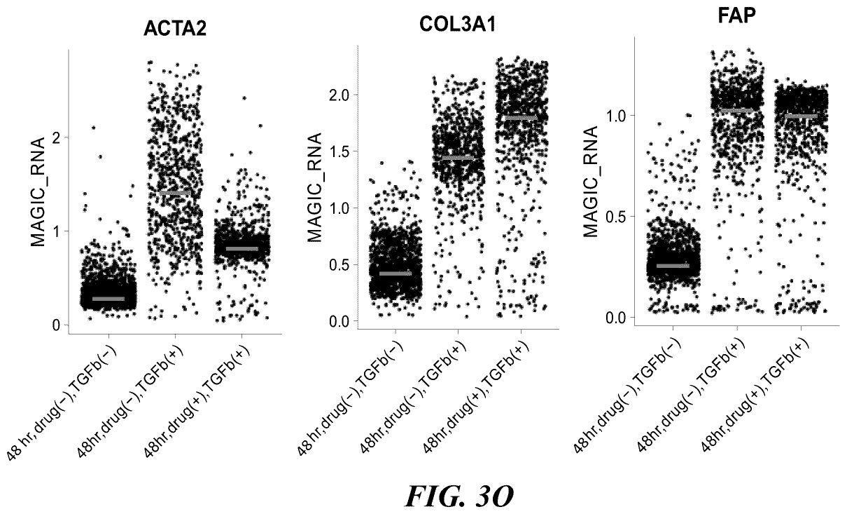

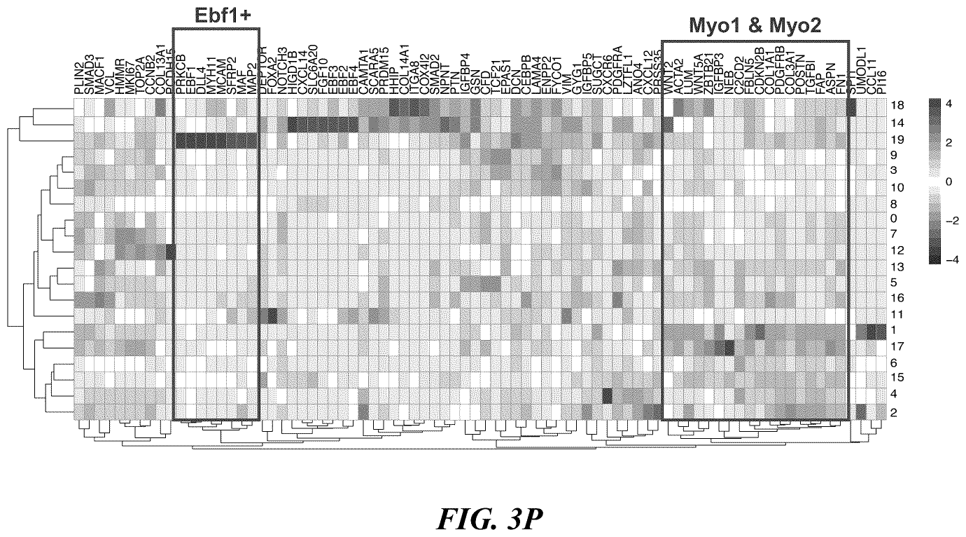

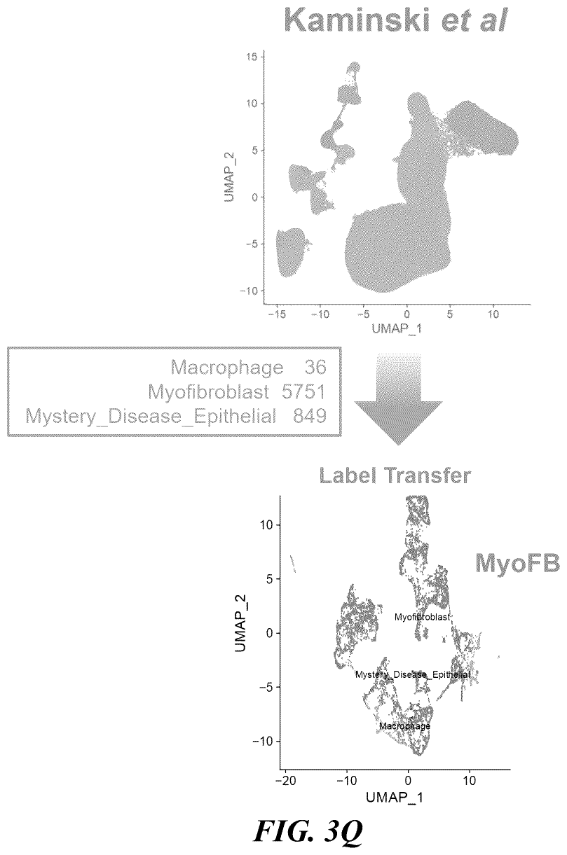

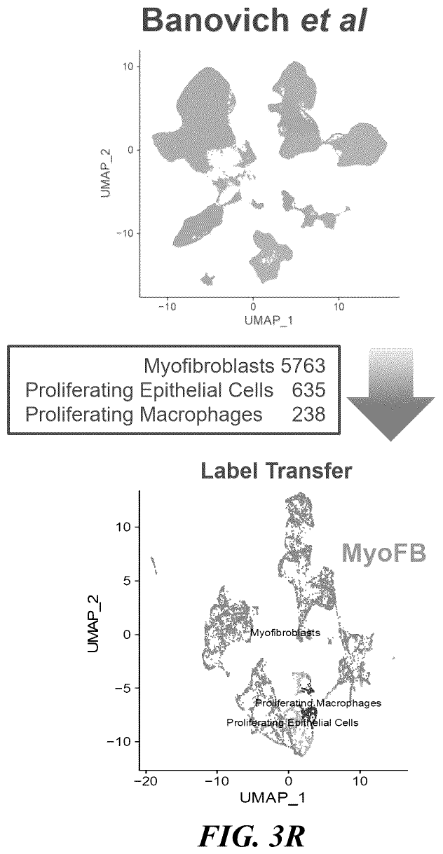

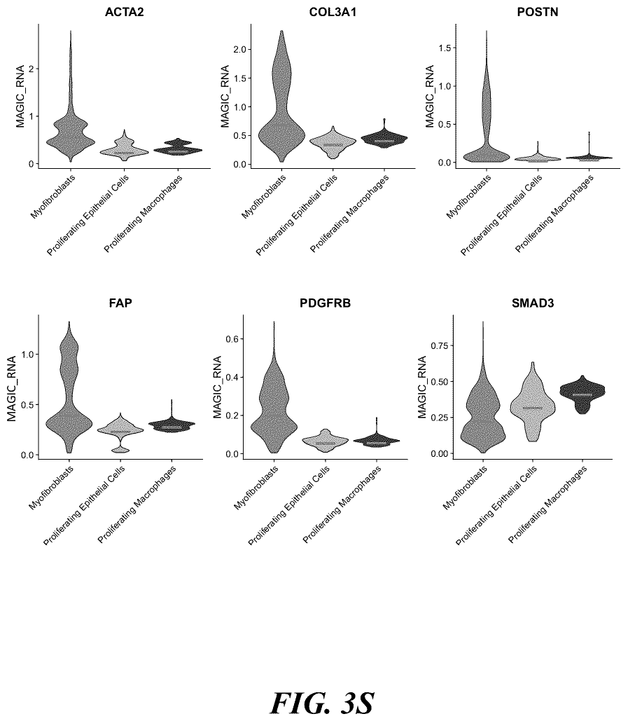

As shown in FIG. 3A, human lung fibroblasts were serum starved and treated with TGFβ for 24-48 hours, and myofibroblasts were simulated. Further analysis was carried out on myofibroblasts (MyoFB). Specifically, cells were thawed and passaged with conventional methods twice a week, with 1:10 for 3 days and 1:15 for 4 days. Cells grew to 70-80% confluency.

For 6 well plates, 150,000 cells were seeded per well and were left to recover overnight before starting experiments. No cells past passage 6 were used for experiments.

After starvation and treatment of TGFβ for 24-48 hours, myofibroblasts were simulated, considering that the morphologies (see FIG. 3B) and the elevation of well-accepted fibrosis markers (see FIG. 3C, FIG. 3F, FIG. 3G, FIG. 3H, FIG. 3I, and FIG. 3J) indicated the myofibroblasts generated after starvation and TGFβ treatment resembled fibroblasts in the state of fibrosis. Specifically, for FIG. 3B, microscope pictures were taken on default settings of bright field illumination. For FIG. 3C, results from PRO-seq (methods specified in other examples) on the Fibronectin 1(FN1) locus, which is a well-accepted fibroblast marker that increases upon starvation and TGFβ treatment. In FIG. 3C, FN1 was shown to produce more nascent RNA in the treated myofibroblasts, which indicated that the myofibroblast (“disease”) state was achieved.

lncRNA transcript expression from the LOC107986083 genomic locus (CORAL) was examined in differentiating fibroblasts in FIG. 3D. Human lung fibroblasts (HLF) and human lung myofibroblasts both exhibited substantial CORAL expression when examined by RNA-Seq in these cell types (graphed in fragments per million mapped fragments [FPM] in FIG. 3D).

Lung FB that have been induced to differentiate into lung MyoFB demonstrated a significant increase in expression of CORAL transcripts. In contrast, human cardiac fibroblasts (HCF), human cardiac myofibroblasts, human dermal fibroblasts (HDF), and human dermal myofibroblast all exhibited very weak/undetectable levels of expression with no evidence of increases in expression level when differentiating from fibroblast cell state to myofibroblast cell state. These results demonstrate cell-type specificity and tissue-type specificity for CORAL. The increase in CORAL expression during lung MyoFB differentiation may indicate a role in the initiation, progression, and/or severity of the fibrosis phenotype induced in this model system.

For qPCR results testing expression of selected fibrosis markers in FIG. 3F, FIG. 3G, FIG. 3H, FIG. 3I, and FIG. 3J, RNA was isolated with the RNeasy RNA extraction kit, and was reverse transcribed using the Quantitect reverse transcription kit. Human TaqMan® primers/probes (also used in other relevant examples described herein) were purchased from Thermo Fisher with the following references: GAPDH, Hs02786624_gl ACTA2, Hs00426835_gl FN1, Hs01549976_ml FAP, Hs00990791_ml COL1A1, Hs00164004_ml COL3A1, Hs00943809_ml POSTN, Hs01566750_ml LZTFL1, Hs00947898_ml. For these genes, qPCR is performed with the Premix Ex Taq (Probe qPCR) (Takara RR390W) master mix according to manufacturer's protocol (Takara Cat. #RR390A). A QuantStudio 6 Pro Thermo Fisher Real-Time PCR system was used, and cycles and temperature settings were set according to the manuals. Data were then exported and analyzed using the RQ method on the Design and Analysis 2 software provided by the manufacturer. From qPCR results from FIG. 3F to FIG. 3J, expression of aSMA, COL1A1, COL3A1, Periostin, Fibronectin all increased and maintained an increase from the initial level of transcription (normalized to a value of 1) for at least 48 hours following TGFβ treatment. These results further demonstrate that the myofibroblast (“disease”) state was achieved and maintained. aSMA protein expression in differentiating MyoFB cells was assessed by Western blot to evaluate the protein levels of this fibrosis marker (see FIG. 3K).

From 24 through 96 hr, aSMA protein levels remained elevated in MyoFB from the basal level found in FB, and aSMA protein levels continued to rise at each time point evaluated. The elevated and increasing expression of this fibrosis marker over a period of differentiation into MyoFB further validates this model of pulmonary fibrosis.

In addition, the spliced annotated lncRNA of LOC107986083 (hsCORAL) was detectable by qPCR in primary human lung fibroblasts (HLF). Specifically, RNA was isolated with the RNeasy RNA extraction kit, and was reverse transcribed using the Quantitect reverse transcription kit. hsCORAL qPCR primers were as follows: Forward: 5′-CACGTGAGCATACTGGGC-3′ Reverse: 5′-GCAGAGTCATCAAAGGGTCG-3′. qPCR was performed with the TB Green Premix Ex Taq (Takara Cat. #RR420W) master mix according to manufacturer's protocol. Cycles and temperature settings from this manual were used on a QuantStudio 6 Pro Thermo Fisher Real-Time PCR system. Data were then exported and analyzed using the RQ method on the Design and Analysis 2 software provided by the manufacturer. As seen in FIG. 3E, expression of lncRNA of LOC107986083 (labeled here as CORAL) increased soon after starvation and initiation of TGFβ treatment (12 hr) and was maintained at an elevated level through 48 hr following initiation of TGFβ treatment.

Subcellular localization of the transcript of LOC107986083 was further investigated. Cell culture and starvation and treatment steps were performed as described above and shown in FIG. 3A, and fractionation was performed with the PARIS™ Kit (Thermo Fisher AM1921) according to manufacturer's protocol. hsCORAL primer used was the same as in FIG. 3E. MALAT1 primers used were as follows: Forward: 5′-GTGCTACACAGAAGTGGATTC-3′, Reverse: 5′-CCTCAGTCCTAGCTTCATCA-3′ (also see Lennox et al., Nucleic Acids Res 2016 Jan 29;44(2):863-77). Subcellular localization of the transcript of LOC107986083 was evaluated by comparing the expression level of the transcript of LOC107986083 in the nucleus and cytoplasm. As shown in FIG. 3L, most of the transcripts of LOC107986083 were observed to be localized in the nucleus.

To further validate the in-vitro model of pulmonary fibrosis, the myofibroblasts generated in vitro were compared to myofibroblasts from public annotated datasets containing samples from patients with pulmonary fibrosis.

First, a single cell atlas was constituted for the myofibroblasts generated in vitro. Pirfenidone, a first-line medicine for pulmonary fibrosis and labeled as “drug” in relevant figures, was used. Specifically, single-cell RNA-Seq with 10× Genomics kit and multiplexing hashtags antibodies were carried out for cells with and without pirfenidone, with and without serum starvation and TGFβ treatment, for different durations (see FIG. 3M for the preparation of myofibroblasts generated in vitro). Briefly, cells from all conditions were collected, counted, treated with hashtag antibodies and prepared for loading on the 10× Chromium with the 10× Genomics 3′ assay kit. Sequencing was performed with a NovaSeq 6000 using the SP 100 cycle kit for a total 600-800 M read depth. Demultiplexing and counting was performed with the CellRanger suite. Cell clustering analysis was then performed using the Seurat R package. Identities of detected clusters were based on label transfer and existing publications (see below). As shown in FIG. 3N to FIG. 3O and FIG. 4J to FIG. 4M, an internal lung myofibroblast single cell atlas was constituted. It was observed that serum starved samples treated with TGFβ displayed increased fibrosis marker expression, while pirfenidone alleviated fibrosis marker expression.

Furthermore, based on their different gene signatures, cell subpopulations relevant for idiopathic pulmonary fibrosis (IPF) were identified in this dataset (see FIG. 3P). “Ebf1+”, “Myo1 & Myo2” in FIG. 3P correspond to the three subpopulations that were identified in Liu et al., iScience. 2021 Jun. 25; 24(6): 102551. The Ebf1+subtype identified a novel class of fibroblasts with a high potential for differentiation. “Myo1” and “Myo2” represented two myofibroblast signatures reported in literature. Therefore, the in-vitro model described here was able to recapitulate these specific in-vivo fibroblast lineages.

Idiopathic Pulmonary Fibrosis (IPF) Cell Atlas is developed as a multi-institutional collaboration to continuously publish datasets presenting in-vivo human disease. Categorical label transfer was performed from Banovich et al. and Kaminski et al. datasets using the TransferData tool in Seurat. To transfer the categorical labels comprising cell identities known from the in vivo scRNA-Seq datasets derived from patient biopsy samples the following method was used. Label transfer between In Vivo patient datasets (reference) and In Vitro model (query) was performed separately for each reference dataset. Expression from each query and reference dataset was preprocessed and normalized in the R environment using Seurat functions. The MAGIC R package was used for data imputation to enhance the signal and counteract the dropout effect of scRNASeq profiling. Label transfer was performed using a proper sequence of functions from the Seurat R package. Anchors between reference and query were obtained using Seurat function FindTransferAnchors using 30 dimensions. Next, data were transferred between datasets using function TransferData (using 30 dimensions), and query cells were annotated with an identity from the reference dataset using Seurat function AddMetaData. As shown in FIG. 3Q and FIG. 3R, labels from IPF Cell Atlas were transferred to an in vitro model described herein. In this manner, information regarding the labeling of cell identities defined in vivo was transferred to in vitro data described herein to infer in vitro cell type within the in vitro examples described herein. As shown in FIG. 3Q to FIG. 3R, Banovich et al. predicted labels and Kaminski et al. predicted labels transferred from the datasets with known cell types identifying most of the cells generated in vitro in FIG. 3M as myofibroblasts. Minority cell labels transferred from the Kaminski et al. dataset to in vitro data described herein include ‘macrophage’ and an undefined epithelial disease ‘Mystery_Disease_Epithelial’. Minority cell labels transferred from the Banovich et al. dataset to in vitro data described herein include ‘proliferating macrophages’ and ‘proliferating epithelial cells’. As shown in FIG. 3S, in vitro marker gene expression described herein was graphed using the transferred labels from the Banovich et al. dataset to indicate range and abundance of expression of each marker gene tested as graphed by Banovich et al. dataset transferred cell type label.

Taken together, the myofibroblasts generated in vitro recapitulated the in vivo human pulmonary fibrosis, and they are a useful tool to test reagents treating lung diseases such as pulmonary fibrosis and pathology related to the development and progression of ARDS.

### Example 3: ASOs Targeting Human LOC107986083

First, various antisense oligonucleotides (ASOs) were generated in silico targeting the RNA transcript of LOC107986083. Then development candidates were selected in silico for screening and synthesis. The selection criteria were: (1) no off-target hybridization; (2) absence of questionable motifs; (3) target accessibility; (4) hybridization free energy (AG); and (5) secondary structures (AG). Several ASO candidates shown in Table 3 were selected for further validation.

TABLE 3
Sequences of ASO candidates targeting transcript of human LOC107986083

| SEQ ID | | | |
|---|---|---|---|
| NO. | Name of ASO | Sequence | Notes |
| 1 | hsCORAL ASO-1 | GGATAATGGTTGGTCA | ASO against human |
| | | | LOC107986083 |
| 2 | hsCORAL ASO-2 | CAAGTAAGCGTGTAGC | ASO against human |
| | | | LOC107986083 |
| 3 | hsCORAL ASO-3 | GTCTGGTAACTATGAG | ASO against human |
| | | | LOC107986083 |
| 4 | hsCORAL ASO-4 | AGATTCATGCAGATGG | ASO against human |
| | | | LOC107986083 |

Notes:
underlined nucleotide is a beta-D-oxy LNA nucleotide

Primary NHLFs (normal human lung fibroblasts) from patient biopsies (Lonza, ref. LZ-CC-2512) were cultured with FGM-2 Fibroblast Growth Medium-2 BulletKit (Lonza, ref. CC-3132). The ASOs were delivered to the cells by transfection (transfection reagent X-tremeGENE, Roche, ref. 6366244001). Briefly, for primary NHLFs grew in 6-well plates with a density of 150,000 cells/well, the final concentration of the ASOs used was 50 nM, and the amount of the ASOs per well was 100pmol. ASOs were diluted in TE buffer with a working dilution of 20 μM.

For each triplicate of 3 wells and accounting for pipetting errors, 16 μL of such working dilution of the ASO, and 6 μL of X-tremeGENE HP DNA Transfection Reagent was mixed respectively into 298 μL of OptiMEM medium gently, which resulted in a total volume of 320 μL with a concentration of 1 pmol/μL. The mixture was incubated at room temperature (15-25° C.) for a minimum of 15 minutes. After incubation, the mixture was added to each well dropwise gently (100 μL of the mixture/well).

The fibrotic phenotype was then induced 24 hours after transfection through serum starvation and TGFβ addition, the cells were collected and evaluated another 24 hours later (see FIG. 4A). Specifically, 24 h after transfection medium was changed to serum-free medium containing 5 ng/mL TGFβ . 24h after TGFβ treatment, cells were collected using Qiazol lysis buffer from RNA extraction kits. If needed, the plates were frozen at −80° C. Specifically, RNA was isolated with the RNeasy RNA extraction kit, and was reverse transcribed using the Quantitect reverse transcription kit. TruSeq® Stranded RNA sequencing libraries were also made, and samples were sequenced on an Illumina HiSeq 4000 PE 150 with an output of about 10-50M reads/sample. RNA-Seq analysis was performed as described above, and barplots for genes of interest were generated to confirm qPCR data.

In the fibrotic phenotype-induced cells, transfection of an ASO (SEQ ID NO: 1) targeting the lncRNA polyA transcript knocked down the expression level of the lncRNA polyA transcript (see FIG. 4B) compared to expression level of the lncRNA polyA transcript in the scrambled ASO (ASO-scr)-treated, fibrotic phenotype-induced control cells as assayed by qPCR.

Expression of one or more transcripts of LOC107986083 in ASO-1-treated fibrotic phenotype induced cells was knocked down to a level of expression similar to that in primary NHLFs not having undergone serum starvation and TGFβ treatment (Control). Such results indicate that expression level of the lncRNA polyA transcript (hsCORAL) was substantially increased upon induction of fibrotic phenotype in the fibroblast (e.g., upon transitioning to myofibroblast), and such increase of the lncRNA polyA transcript (CORAL) could be reduced or even reversed to the pre-induction state (e.g., fibroblast state) by treating with ASO-1. In this experiment, qPCR primers used were as follows: Forward: 5′-CACGTGAGCATACTGGGC-3′ Reverse: 5′-GCAGAGTCATCAAAGGGTCG-3′. Cycles and temperature settings were adopted from manufacturers' protocols.

Expression of fibrotic marker genes were further profiled upon modulation of expression of one or more transcripts of LOC107986083. In the fibrotic phenotype-induced cells, expression of several profiled fibrotic marker genes could be reduced upon transfection of ASO-1 (SEQ ID NO: 1), in statistically significant levels of decrease (see FIG. 4C to FIG. 4I, FIG. 4L, and FIG. 4M) compared to cells treated with scrambled ASO control (ASO-Scr). As seen in FIG. 4C, expression of ACTA2 was significantly decreased by ASO-1 treatment. As seen in FIG. 4D, expression of COL1A1 was decreased by ASO-1 treatment. As seen in FIG. 4E, expression of COL3A1 was significantly decreased by ASO-1 treatment. As seen in FIG. 4F, modulation of expression of FAP was not seen to reach a significant difference by ASO-1 treatment. As seen in FIG. 4G, expression of FN1 was modulated by ASO-1 treatment. As seen in FIG. 4H, expression of POSTN was decreased by ASO-1 treatment. Responses shown in FIG. 4B-FIG. 4H were assayed by qPCR. As seen in FIG. 4I, ASO-1 treatment reduced expression of selected markers of fibrosis (ACTA2, COL1A1, COL3A1, FAP, FN1, and POSTN) which were elevated following induction to MyoFB as assayed by RNA-Seq. Reductions in the levels of five of the fibrosis markers following ASO-1 treatment reached significance (ACTA2, P-value: 0.0559; COL1A1, P-value: 0.0319; COL3A1, P-value: 0.0103; FAP, P-value: 0.0257; FN1, P-value: 0.00231).

Taken together, these results demonstrate that ASO-1 treatment targeting hsCORAL could reduce expression of the target lncRNA as well as several pro-fibrotic genes, which are potential downstream genes modulated by the target lncRNA. Consequently, the ASO-1 treatment targeting hsCORAL could have an anti-fibrotic impact on a lung cell model of pathological fibrosis.

Ontology analysis was performed for genes that were up-regulated or down-regulated in response to treatment of the ASO (SEQ ID NO: 1) (see FIG. 4N and FIG. 4Z). Briefly, classical Gene Ontology Enrichment (GOE) was performed using the R package ClusterProfiler, which identified biological processes and programs significantly over- or under-activated in a given condition. Input data is a set of genes (e.g., differentially expressed genes, filtered by some pre-defined thresholds on fold change and p Value). The output list of significantly enriched GO terms are displayed using treemap plots (from R packages treemap and rrvgo). The treemaps cluster together GO terms with high semantic similarity, representing similar or overlapping biological processes. Single-nucleus RNA-Seq (snRNA-Seq) was used to generate the data for the ontology analysis. This ontology analysis and the results derived from it were used to constitute an internal single cell atlas for use in interpreting sets of genes and gene expression data while making use of the GO system of classification for the ASO-treated myofibroblast cells. Genes were assigned to a set of predefined bins depending on their functional characteristics and GO term labels were applied to the bins. UMAP analysis of snRNA-Seq results comparing FB to MyoFB samples in FIG. 4J indicated that FB and MyoFB samples separate, and that FB and MyoFB-ASO_Scr-treated cells also separate. This confirms a difference in cells states between FB and MyoFB. The distribution of untransfected MyoFB mostly overlaps with MyoFB-ASO_Scr-transfected cells showing that ASO_Scr transfection does not substantially affect cell state. Cell cycle analysis revealed a key difference between the FB and MyoFB states, which appears to be the proportion of quiescent to dividing cells: the majority of untransfected MyoFB and Scr MyoFB are in G1 while FB cells are dividing more actively. This result was supported by separated analysis of these cell types using FACS. UMAP analysis of snRNA-Seq results comparing MyoFB-ASO_Scr-treated cells to MyoFB-ASO-1-treated cells in FIG. 4K indicated that ASO-1-transfected cells grouped distinctly from ASO_Scr-transfected cells and showed a slight increase in the proportion of proliferating to quiescent cells. FIG. 4L showing graphs of expression level from snRNA-Seq data from experimental groups described herein confirmed that MyoFB cells show an increase in expression of fibrosis-related genes relative to FB cell and that MyoFB-ASO-1-treated cells show a significant decrease in several fibrosis markers compared to MyoFB-ASO_Scr-treated cells.

FIG. 4M further confirms that reduction of fibrosis marker expression following ASO-1 treatment in MyoFB. Groups of gene upregulated in response to ASO-1 treatment were identified as were groups of genes downregulated by ASO-1 treatment as shown in FIG. 4N. 266 genes were identified as being significantly upregulated in response to ASO-1 treatment and 398 genes were identified as being significantly downregulated in response to ASO-1 treatment. GSEA terms with the highest enrichment for upregulated (upper left box) and downregulated (lower left box) are listed in FIG. 4N.

In the primary NHLF cells in which the fibrotic phenotype has been induced, treatment with ASO-1 (SEQ ID NO: 1) resulted in a knockdown of lncRNA polyA transcript (see FIG. 40) compared to expression level of lncRNA polyA transcript of scrambled ASO (ASO-scr)-treated control, while ASO 2, ASO-3, and ASO-4 (corresponding to SEQ ID NOs: 2-4 respectively) did not demonstrate a statistically significant knockdown of expression of human lncRNA transcripts from LOC107986083. FIG. 4P-FIG. 4S show a comparison of the effects of various ASOs on modulating elevated transcription of selected fibrotic markers genes in primary NHLF cells in response to serum starvation and TGFβ treatment. As seen in FIG. 4P, ASO-1, -2, and -3 treatment resulted in a reduction of the elevated expression of human COL1A1 in response to serum starvation and TGFβ treatment in comparison to a negative control scrambled ASO (MyoFB ASO-Scr). As seen in FIG. 4Q, ASO-1, -2, and -4 treatment resulted in a reduction of the elevated expression of human FAP in response to serum starvation and TGFβ treatment in comparison to MyoFB ASO-Scr. As seen in FIG. 4R, ASO-1, -2, -3, and -4 treatment resulted in a reduction of the elevated expression of human FN1 in response to serum starvation and TGFβ treatment in comparison to MyoFB ASO-Scr. As seen in FIG. 4S, ASO-1,-2, and -4 treatment resulted in a reduction of the elevated expression of human POSTN in response to serum starvation and TGFβ treatment in comparison to MyoFB ASO-Scr. ASO-1 demonstrated the strongest knockdown of POSTN of the ASOs tested and this reduction in expression level reached a comparable level of POSTN expression as seen in control fibroblasts in which the fibrotic phenotype was not induced. ASO-1 was the only candidate ASO tested to on hsCORAL that produced a significant hsCORAL knockdown and a reduction of some molecular markers of fibrosis. As seen in FIG. 4T-FIG. 4V, treatment with all four ASOs (ASO-1 to ASO-4 was shown to decrease expression of LTBP2, THBS2, and CTHRC1 compared to ASO-Scr treatment in the myofibroblast model. ASO-1 treatment of MyoFB resulting in alteration of expression of an unbiased IPF gene set derived from in vivo and in vitro samples. Singscore evaluating the samples against the human IPF gene signature was significantly reduced following ASO-1 treatment indicating that a pathological state of the MyoFB cells was significantly altered and was determined to move in a direction more similar to that of FB cells (FIG. 4W). This indicates that ASO-1 treatment reduces molecular pathological features representing IPF.

ASO-1 was tested for dose-response in its effect on hsCORAL expression and expression of select markers of fibrosis (FIG. 4X-FIG. 4Y). FIG. 4X shows the results of a dose-response experiment in which various concentrations of ASO-1 (between 0 nM to 50 nM) in cells of the in-vitro model for pulmonary fibrosis used in this example are displayed as plotted in dose-response curves. IC₅₀ (the concentration at which ASO-1 exerted half of its maximal inhibitory effect) was calculated to be 3.17 nM. RC₅₀ coefficient (the concentration at which ASO-1 exerted its effect to deplete or knockdown expression of a fibrosis marker by 50%) was calculated to be 8.1 nM for COL3A1, 9.16 nM for FN1, and 3.49 nM for POSTN. These results demonstrate that ASO-1 treatment produces a dose-dependent reduction in hsCORAL expression. These results also demonstrate that ASO-1 treatment produces a dose-dependent reduction in expression of select markers of fibrosis including COL3A1, FN1, and POSTN. These results also demonstrate that ASO-1 treatment produces a dose-dependent reduction in expression of both hsCORAL and select markers of fibrosis including COL3A1, FN1, and POSTN. FIG. 4Y shows dose-responsive fibrotic gene marker expression to ASO-1 treatment in the in-vitro model for pulmonary fibrosis used in this example. All tested markers of fibrosis (ACTA2, COL1A1, COL3A1, FAP, FN1, and POSTN) responded to increasing dosage of ASO-1 treatment with increasing reductions in expression level. RC₅₀ coefficient was calculated for ACTA2 (7.23 nM; p-value=0.0128), COL1A1 (9.6 nM; p-value=0.012312), and COL3A1 (9.68 nM; p-value=1.23e⁻⁰³)

ASO-1 was tested for effect on modulating various molecular and cellular pathways in the in-vitro model for pulmonary fibrosis used in this example (see FIG. 4Z). ASO-1-treated primary NHLF cells following serum starvation and treatment with TGFβ were assayed for differentially expressed genes (DEG) and results were compared with scrambled ASO-treated primary NHLF cells following serum starvation and treatment with TGFβ. In this gene set enrichment analysis, GO terms on genes that are differentially expressed were used for categorization. Assayed genes were grouped into GO terms as categorized by involvement in a shared molecular or cellular pathway. Fibrosis-related GO terms are indicated with (F) and immune-related GO terms are indicated with (I). Ranked GO terms are listed according to gene count. ‘Count’ is the gene count, which is the number of genes enriched for a particular GO term. ‘GeneRatio’ is the percentage of DEG in the given GO term. hsCORAL knockdown by ASO-1 downregulated extracellular matrix-related pathways and certain immune response-related pathways (e.g., leukocyte activation and cell adhesion). These results indicate which cellular functions are likely to be modulated in response to ASO-1 treatment. These results also indicate which cellular functions are likely to be modulated in response to ASO-1 treatment that downregulates the expression of one or more genes relating to particular GO term and reduces a biological function represented by that GO term.

### Clustering of Gene Expression

Effects of ASO-1 treatment on gene expression in primary NHLF cells in which the fibrotic phenotype has been induced was tested using RNA-Seq for a comparative analysis of quantitative measures of expression changes. ASO treatment at either 50 nM or 20 nM concentrations was used for analysis. Analysis was performed on genes with average(FPM)>10 (N=10179) and Euclidean distance of log scaled_FPM was used as a metric of measurement. Pairwise distance between each gene were calculated for hierarchical clustering within a latent space with a cluster choice of optimization of average silhouette width. ASO-1 treatment was found to downregulate several clusters of genes as shown in FIG. 4AA. Included in Cluster 2 were COL1A1 and FAP. Included in Cluster 6 were COL3A1, FN1, POSTN, and ACTA2. Clusters 2 and 6 were the most downregulated by ASO-1 treatment of the identified clusters. Clusters 2 and 6 are enriched for ECM-related genes indicating that ASO-1 knockdown of CORAL modulates expression of genes relating to the function of extracellular matrix.

### Cluster Refinement and Gene Set Enrichment Analysis

To refine the analysis further, a cluster-based target engagement panel (TEP) was derived. In this TEP, the most downregulated cluster for ASO-1 treatment on knockdown of CORAL expression was selected. CORAL ASO-1 (Cluster 6): n=287 was selected. As controls, the most upregulated and the most downregulated clusters following ASO-Scr treatment were selected.

Within cluster 6, ASO-1-sensitive genes were enriched for GO terms relating to ECM and actin cytoskeleton terms.

GSEA was performed on data derived from 50 nM treatment of ASO-1 vs. 50 nM treatment of ASO-Scr on primary NHLF cells in which the fibrotic phenotype has been induced. Results from GSEA are shown in FIG. 4Z in which ASO-1 treatment downregulated several immune response-related pathways according to listed GO terms such as those involving leukocyte activation or cell adhesion. Notably, genes related to myeloid leukocyte activation were downregulated following treatment with ASO-1. Subselected genes in the “GO:0002274 myeloid leukocyte activation” term were examined further and visually checked to verify that they were not also strongly upregulated by ASO-Scr treatment. This verification procedure is similar as to how the IPF gene signature data in Example 7 is analyzed in comparison to a control using ASO-Scr treatment. From this subselected list of genes, 16 genes were further selected as a short list for further evaluation (ADAM10, CLU, DOCK2, FER, FOXF1, FOXP1, GAB2, IL4R, JAK2, LGALS9, LRRK2, MYO18A, NDRG1, PRKCE, RORA, and THBS1). As shown in FIG. 4AB to FIG. 4AC, all 16 of these genes were downregulated following treatment with ASO-1 at 20 nM and at 50 mM and were not sensitive to ASO-Scr upregulation. Expression curves from IC₅₀ datasets for the short list of myeloid leukocyte activation genes plotted using ASO-1-treated cells were visually compared to those derived from ASO-Scr-treated cells. The inflection point of the curve occurred earlier for the ASO-1-treated cells than for the ASO-Scr-treated cells.

As shown in FIG. 4AD-FIG. 4AF, dose-responses were calculated for the short list of myeloid leukocyte activation genes for ASO-1 treatment in comparison to ASO-Scr treatment.

As shown in FIG. 4AG, the HLF cells grown in vitro used in this example were examined under bright field microscopy at various time periods following ASO treatment (0 hr, 24 hr, 48 hr). Treatment with ASO-1, ASO-2, ASO-3, ASO-4, or ASO-Scr did not produce any obvious signs of toxicity through bright field microscopy analysis.

### Interferon-Stimulated Genes (ISG)

An ISG gene set has been derived for FB and MyoFB in which the cell types were treated with IFNγ. As this gene set has been analyzed and compared to existing gene sets, genes shared between IFNγ treatment and TGFβ treatment are enriched for GO terms relating to leukocyte migration (e.g., cell chemotaxis, leukocyte migration, leukocyte chemotaxis, monocyte chemotaxis). Further tested indicated that ASO-1 treatment in fibroblasts downregulated a subset of ISGs and was not found to upregulate any gene member of this subset. ISGs downregulated by ASO-1 treated were found to be enriched for leukocyte activation—and proliferation-related GO terms.

### Example 4: Further Validation of ASOs Against Transcript of Human LOC107986083

PRO-seq of primary human lung fibroblasts treated with ASOs of interest is performed. PRO-seq experimental approach and its bioinformatic analysis is as described herein. After bioinformatic analysis is performed, dysregulation of nascent RNA on key fibrosis marker genes (e.g., ACTA2, POSTN, FAP, COL1A1, COL3A1, and FN1) and dysregulation of nascent RNA at the LOC107986083 locus are observed. Immortalized human lung fibroblasts are included as well.

Enhancer status of LOC107986083 locus upon ASO treatment is evaluated. Cleavage Under Targets & Release Using Nuclease (Cut&Run) was performed with CTCF (negative control), H3K4me3 (using the CUTANA™ Cut&Run kit and antibodies recommended by the manufacturer). Single-nucleus ATAC-Seq (snATAC-Seq) was also performed to enable quantification of chromatin accessibility in single nuclei. H3K27ac ChIP/C&R analysis is performed.

FACS profiling of extracellular markers (e.g., α-SMA, FAP) with or without treating of the ASOs described herein are performed. Proteins of ECM markers such as ACTA2 and FAP are quantified on the surface of treated pulmonary cells by staining and flow cytometry are planned to validate the effect of the ASOs described herein (e.g., SEQ ID NO: 1) Specifically, HLFs transfected with target ASOs or Scramble, treated with serum starvation and TGFβ or not, are collected. The anti-alpha smooth muscle Actin antibody [1A4](Abcam ab7817) is used for the detection of the alphα-SMA protein encoded by the ACTA2 human gene, at a concentration of 1.137 μg/mL as suggested by the manufacturer. Anti-Fibroblast activation protein, alpha antibody (Abcam ab28244) is used for the detection of protein encoded by the FAP human gene.

FACS is performed according to the manufacturer's protocol (https://www.abcam.com/protocols/indirect-flow-cytometry-protocol) on a Beckman Coulter Gallios flow cytometer. Results are read and analyzed with the Kaluza acquisition software. A change in extracellular levels of alphα-SMA (ACTA2) and FAP is quantified upon knocking down LOC107986083.

CUT&RUN of H3K27ac with the Abcam antibody ab4729 is performed according to the protocol in the “mammalian cells” rubric in Skene et al., eLife 2017;6:e21856 DOI: 10.7554/eLife.21856. It is expected that a dysregulation of H3K27ac marks on and around the LOC107986083 locus is observed. A difference in H3K27 levels at the regulatory regions of key fibrotic genes is also observed.

### Example 5: ASOs Against Transcript of Mouse Homolog of LOC107986083

First, a mouse homolog of LOC107986083 was identified. Specifically, whole-lung RNA-Seq with about 200 million reads/sample using 1-year old mouse lung tissues was performed. Mapping was performed as described above with the mm39 mouse genome as reference. A transcribed region at the 3′ end of mouse Lztf11 transcript was observed (see FIG. 5A)

Two candidate ASOs shown in Table 4 were designed and selected for further testing.

TABLE 4
Sequences of ASO candidates targeting transcript of mouse homolog of
LOC107986083

| SEQ | | | |
|---|---|---|---|
| ID | | | |
| NO. | Name of ASO | Sequence | Notes |
| 5 | mmCORAL ASO-1 | ATGATGTGCAACAGGC | ASO against mouse homolog of |
| | or G6 | | LOC107986083 |
| 6 | mmCORAL ASO-2 | CCCTGTATTACCCTGC | ASO against mouse homolog of |
| | or G9 | | LOC107986083 |

Notes:
underlined nucleotides are modified by beta-D-oxy LNA

Immortalized MLg mouse lung fibroblast cell line was cultured in DMEM supplemented with 10% FBS. Transfection was performed with polymer reagent (X-tremeGENE, Roche, ref. 6366244001). Specifically, RNA was isolated with the RNeasy RNA extraction kit from Qiagen (ref. 74106), and was reverse transcribed using the Quantitect reverse transcription kit from Qiagen (ref. 205311). Shown in FIG. 5B to FIG. 5G are the elevation in expression levels of the homolog of LOC107986083 and classic fibrotic markers in response to serum starvation and TGFβ treatment. Also shown in FIG. 5B to FIG. 5G are the effects of mmCORAL ASO-1 (G6 or mmASO-1 and mmCORAL ASO-2 (G9 or mmASO-2) treatment indicating effective knockdown of expression of the mouse homolog of LOC107986083 and down-regulation by these two ASOs (G6 and G9) of expression of several induced fibrotic markers.

After investigating the ASOs with the immortalized MLg mouse lung fibroblast cell line, primary mouse lung fibroblasts were used to further validate the selected ASOs. Specifically, primary mouse lung fibroblasts were isolated directly from 5 month old C57BL/6 mice cultured in DMEM supplemented with 10% FBS and 1% P/S. Transfection was performed with polymer reagent (X-tremeGENE™, Roche, ref. 6366244001). The following TaqMan® probes are purchased from Thermo Fisher and used with the TaqMan® method described above: Acta2 Mm00725412_s1 Collal Mm00801666_gI Col3al Mm00802296_gl Postn Mm01284913_gl Fn1 Mm01256744_ml Fap Mm01329177_ml Gapdh Mm99999915_gl mmCoral primers are as follows and are used with the TB Green SYBR method mentioned above: mmCoral_fwdl: 5′-AGAACTTGAAGCTGTCAGGG-3′, mmCoral_revl: 5′-TGCATGTTGAAGACAGCACT-3′. As shown in FIG. 5H-FIG. 5J, the elevated expression of Acta2, Col3al, and Fn1 were reduced by the two ASOs (G6 and G9). SEQ ID NO: 6 was shown to be more efficient against fibrosis in vitro and is used in RNA-Seq studies and an animal study.

In addition, primary mouse lung fibroblasts isolated directly from 24 month old C57BL/6 mice were tested. The fibroblasts were cultured in DMEM supplemented with 10% FBS and 1% P/S. Transfection was performed with polymer reagent (X-tremeGENE™, Roche, ref. 6366244001). Specifically, RNA was isolated with the RNeasy RNA extraction kit from Qiagen (ref. 74106), and was reverse transcribed using the Quantitect reverse transcription kit from Qiagen (ref. 205311). As shown in FIG. 5K to FIG. 5N, the expression of mouse homolog of LOC107986083 and the elevated expression of Acta2, Col3al, and Fn1 in mice aged 24 months were reduced by the two ASOs.

The results demonstrate a reduction in expression of fibrosis-related genes in young (5 month old) primary mouse lung fibroblasts. The results also demonstrate a more pronounced reduction in expression of fibrosis-related genes in old (24 month old) primary mouse lung fibroblasts. As shown in FIG. 5O and FIG. 5P, the expression of several fibrosis markers is downregulated in mouse primary lung fibroblasts following treatment with mmASO-2 compared to treatment with ASO-scr. In young mouse lung fibroblasts, Collal, Col3al, and Postn showed significant reductions in expression (see FIG. 5O). In old mouse lung fibroblasts, Collal, Col3al, and Fn1 showed significant reductions in expression (see FIG. 5P). In FIG. 5Q, the left chart shows that mmASO-2 treatment reduced expression of markers from a IPF gene signature in young primary mouse lung fibroblast cells and the right chart shows that mmASO-2 treatment reduced expression of markers from the IPF gene signature in old primary mouse lung fibroblast cells. The mouse IPF gene signature assayed in FIG. 5Q was developed through meta-analysis of existing RNA-Seq datasets assaying mouse pulmonary cells under specific conditions and by evaluation of a bleomycin induced pulmonary fibrotic disease dataset generated in house and publicly available bleomycin datasets (see Example 7). As shown in FIG. 5R, mouse CORAL knockdown by treatment with mmASO-2 downregulated genes grouped in several immune-response pathways. RNA-Seq analysis was performed on mouse lung fibroblasts (from 24 month old mice). FIG. 5R depicts genes that are downregulated following mmASO-2 treatment as compared to ASO-Scr treatment. GSEA was performed with the gseGO function of the cluster Profiler package implemented in Bioconductor in R. The top 40 GO terms representing categories of genes showing statistically significant, concordant differences between the two datasets tested are shown in rank order in FIG. 5R. As shown in FIG. 5S, mouse CORAL knockdown by treatment with mmASO-2 downregulated genes grouped by several leukocyte migration-related terms. All members of this manually selected grouping of leukocyte-related GO terms were significantly enriched for genes showing statistically significant, concordant differences between the two datasets tested. As shown in FIG. 5T-FIG. 5U, mouse CORAL knockdown by treatment with mmASO-2 downregulated expression of several immune-related genes. Downregulation of Adaml0, Fer, Gab2, Jak2, Myo18a, Ndrg1, Prkce, and Rora were significant comparing mmASO-2-treated mouse lung fibroblast (MLF) cells in vitro to ASO-Scr-treated cells.

RNA-Seq was performed on primary mouse lung fibroblasts treated with SEQ ID NO: 6. RNA-Seq was performed on a NovaSeq 6000 with ribodepleted Illumina TruSeq® library preparation 150 PE. Samples were sequenced at an expected depth of up to 40M reads/sample. Related bioinformatics analysis pipeline for bulk RNA-Seq was as described above.

### Example 6: In Vivo Validation of ASOs in a Pulmonary Fibrosis Mouse Model

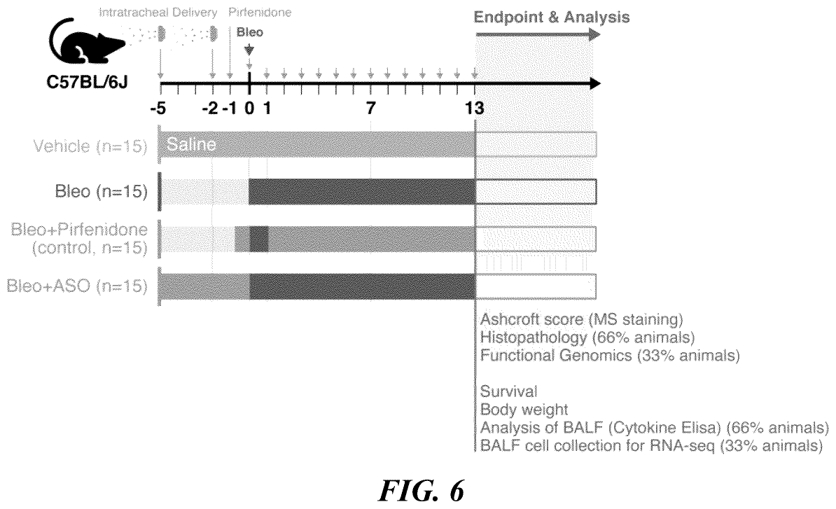

A pulmonary fibrosis (e.g., idiopathic pulmonary fibrosis) mouse model is established by an application of bleomycin, as shown in FIG. 6. Specifically, a single 1.5 U/kg dose (O.P. or I.T.) of bleomycin elicits an initial lung inflammation (D0-D7), which subsequently results in progressive lung fibrosis (D7-D28). As a positive control, pirfenidone, a medication that has been used for the treatment of pulmonary fibrosis (e.g., idiopathic pulmonary fibrosis) in the current practice, is administered twice daily orally (at a dosage of 100 mg/kg). In the experimental group, ASOs, at 100 μg/mouse, are administered on day -5 and day -2 in an aerosol format. Various endpoints and the corresponding analysis are monitored across different groups. For example, assuming 12 animals survive, 8 animals are used for histology and 4 are for RNA-Seq analysis. Day 13 is the endpoint of the study. The ASOs are detected in liver and unused lung lobes at the end of the study.

### Test Groups

Four test groups of mice were assays in this example. Group 1 mice received no bleomycin treatment and served as a negative control. Group 2 mice received bleomycin treatment and aerosol administration of vehicle and serve as a negative control of induced lung inflammation and lung fibrosis. Group 3 mice received bleomycin treatment and administration of a therapeutic ASO (e.g., mmASO-2). Group 4 mice received bleomycin treatment and administration of a known therapeutic serving as a positive control (e.g., pirfenidone). A sufficient number of animals were tested for each group for reproducibility of results and statistical analyses of the data. During the study and at the endpoint, animals were assayed for survival, body weight, analysis of bronchoalveolar lavage fluid (BALF) including ELISA assays of select cytokines. At the endpoint of the study, surviving animals were assayed with various tests of functional genomics and scoring of immune system function and pulmonary fibrosis.

### ASO Administration

A therapeutic ASO directed against the mouse homolog of LOC107986083 (e.g., mmASO-2) was administered according to the study protocol. Group 3 mice receiving administration of a therapeutic ASO received two administrations that were spaced apart by 3 days. 100 μg of ASO/mouse/administration was given by intratracheal microspray in an aerosol formulation on Day -5 and on Day -2. This study protocol was designed to assay a prophylactic response of ASO treatment in mice to bleomycin-induced lung inflammation and lung fibrosis. Bleomycin administration was given on Day 0.

### Pirfenidone Administration

Pirfenidone was administered to Group 4 mice starting at Day -1 and was continued twice daily until the endpoint of the study. Pirfendone was administered via oral galvage at a dosage of 100 mg/kg of body weight (100 μL/dose/BID).

### Immune Cell Numbers

Brochoalveolar lavage (BAL) was used to assess immune cell numbers present in mice of the various test groups. FIG. 7A shows that immune cell numbers in the lungs of mmASO-2-treated mice (Group 3) are not significantly increased. FIG. 7A also shows that Group 3 mice do not significantly change the proportion of types of lung immune cells present following administration of mmASO-2 compared to Group 2 mice. Group 4 mice showed a significant reduction in number of macrophages, neurotrophils, and lymphocytes compared to Group 2 mice.

### Weight Loss

FIG. 7B shows that Group 3 mice demonstrated significant weight loss clearly evident by Day 5 through to the end of study. This was the only test group to demonstrate significant body weight loss. Liver and lung weight were tested at end of study for each test group and also compared as a ratio to total body weight at end of study. FIG. 7B also shows that prophylactic treatment with mmASO-2 in Group 3 animals produced a significant increase in lung to body weight ratio compared to Group 2 animals. This increase in lung to body weight ratio is an expected component of the mouse bleomycin model and was found to be slightly alleviated in Group 4 animals. Lung weights were compared between Group 1-4 and Group 4 was found to have a significant reduction in lung weight compared with Group 2. Group 2 and Group 3 did not demonstrate significant differences in lung weight. Liver weight and liver to body weight ratio was compared between Groups 1-4. There were no demonstrated significant changes in liver to body weight ratio between any of the groups indicating the absence of any particular liver toxicity in all groups tested.

### Expression of Markers Using RNA-Seq

RNA-Seq analysis was performed using bulk lung tissue of each test group at the study endpoint. Selected markers of fibrosis (e.g., Acta2, Collal, Col3al, Fap, Fn1, and Postn) were assayed. GSEA analysis of RNA-Seq data from bulk mouse lung tissue of mmASO-2-treated mice compared to ASO-Scr-treated mice (FIG. 7C). GO terms showing significantly enrichment are listed for these groupings of downregulated genes. The results indicate several GO terms involving leukocyte migration, leukocyte adhesion, and infiltrate forming indicating that mmASO-2 treatment downregulates genes grouped as having roles in immune response. A select fraction of GO terms listed in FIG. 7D indicate specific GO pathway enrichment including several terms relating to ECM. Grouping of test results into GO terms and analysis of the data indicated that treatment with mmASO-2 resulted in the downregulation of many genes related to immune function. In particular, GO terms relating to leukocyte migration, adhesion, and infiltrate formation were strongly represented in groups of mmASO-2 downregulated genes. A subset of mouse orthologs of a gene set derived from HLF Fb treated with IFNγ were profiled for their fold changes in Group 2, 3, and 4 animals. The vast majority of this gene set was upregulated in Group 4 animals while a subset of the gene set was downregulated following mmASO-2 treatment in Group 3 animals. Further analysis of RNA-Seq data and GO terms assigned to DEG indicated that mmASO-2 treatment in Group 3 animals resulted in the downregulation of numerous genes grouped by GO terms relating to extracellular matrix.

An anti-ARDS proof-of-concept signal was determined by the use of CORAL ASO targeting. FIG. 7E shows analysis of immune genes (Clu, Dock2, Fer, Gab2, Lgals9) for the groups tested (Group 1: Control, Group 2: Bleo, Group 3: Bleo+mmASO-2, Group 4: Bleo+pirfenidone). FIG. 7F shows putative pathway dependent target genes tested in vitro in human and in vivo in mouse indicating an anti-ARDS signal using treatment with ASO targeting of CORAL. Both DOCK2/Dock2 and LGALS9/Lgals9 were downregulated following ASO treatment demonstrating cross-species conservation of effects of ASO targeting of CORAL in models of ARDS.

### Cytokine Expression

Various cytokines were assayed in bulk lung tissue of each test group at the study endpoint using ELISA. Cytokines tested were IFNγ, IL-1b, IL-2, IL-4, IL-5, IL-6, KC/GRO, IL-10, IL-12p70, TNFα, IL-9, IL-15, MIP-2, IL27p28/IL-30, IP-10, IL-33, MIP-la, MCP-1 and IL-17A/F. As shown in FIG. 7G, IL-1b, MCP-1, MIP-la, IP-10, and MIP-2 demonstrated significant increases in Group 3 animals compared to Group 2 animals. IL-1b, MCP-1, MIP-la, IP-10, and MIP-2 serves as chemokines involved in leukocyte infiltration. These cytokines may be increased as a compensatory reaction to the lack of infiltrated leukocytes in the lungs of Group 3 animals.

### Plasma Biochemistry

Typical plasma biochemistry markers (e.g., blood urea nitrogen, creatine, phosphorous, calcium, total protein, albumin, globulin, ALT, AST, ALP) were assayed for all test groups and were found to reside in the normal range with no toxicity observed. Normal ranges plasma biochemistry markers in mice for comparison to test results were obtained from Charles River C57/BL/6 mice datasheet information (Charles River C57BL/6NCtr Data —Clinical Chemistry).

### Leukocyte Infiltration and Lung Fibrosis

As shown in FIG. 7H, mmASO-2 treatment showed no significant impact on fibrosis score (e.g., increased collagen score and average Ashcroft score). However, examining mixed cells by hematoxylin and eosin (H&E) staining in infiltrate indicated a significant reduction in immune cell infiltrates using histology based-scoring. As shown in FIG. 7I, mmASO-2 treatment showed significant inhibition of immune cell infiltrates with inflammatory cell aggregates not observed in Group 3 animals. Group 1: The average Ashcroft score for this sample was 0; the Group 1 mean was 0.2. No histologic lesions are captured. All airways are open and alveolar walls are thin. An example blood vessel (BV) and bronchiole (Br) are indicated. Group 2: The average Ashcroft score for this sample was 3.8; the Group 2 mean was 3.6. An area of fibrous mass formation (black arrows), comprised of pale eosinophilic (pink) matrix and infiltrates to aggregates (**) of mixed inflammatory cells (primarily lymphocytes), is captured. An example blood vessel (BV) and a bronchiole (Br) are indicated. Group 3: The average Ashcroft score for this sample was 3.6; the Group 3 mean was 3.8. Fibrous masses (black arrows) are seen throughout the section, with minimal mixed inflammatory cell infiltration (**). Inflammatory cell aggregates are not observed. An example blood vessel (BV) and a bronchiole (Br) are indicated. Group 4: The average Ashcroft score for this sample was 3.0; the Group 4 mean was 3.1. A fibrous mass (black arrows) containing infiltrates and aggregates of mixed inflammatory cells (**) affects much of the section. An example bronchiole (Br) and a blood vessel (BV) are indicated.

In this example, administration of oropharyngeal bleomycin induced histopathologic lesions typical for this model, including pulmonary fibrosis with mixed inflammatory cell infiltration/aggregate formation. Prophylactic treatment with an ASO targeting one or more lncRNA polyA transcripts of the mouse homolog of LOC107986083 exhibited efficacy in the reduction of inflammatory cell infiltrate severity but did not significantly reduce detected levels of fibrosis as compared to vehicle treatment. Treatment with the reference positive control compound pirfenidone produced slight, non-significant reductions in the severity of pulmonary fibrosis in this study.

Excessive organ infiltration of inflammatory monocytes and macrophages is a hallmark pathological feature of severe ARDS and severe COVID-19/ARDS. ASO-treated mice in this bleomycin study presented with a decrease of immune cell infiltration in the lungs. This was observed through i) histopathological scoring (infiltrates not visually observed in ASO-treated mice), ii) BAL differential counts (leukocyte numbers in the fluid were not increased), iii) cytokine ELISA from bulk lung tissue (IL-1b, MIP-la, IP-10, MCP-1, and MIP-2 are all significantly increased, and iv) RNA-Seq analysis (GO terms related to leukocyte adhesion and migration were downregulated). These results demonstrate that the CORAL locus is a potential regulator of leukocyte adhesion and/or infiltration in ARDS.

### Example 7: Idiopathic Pulmonary Fibrosis (IPF) Gene Sig-Nature

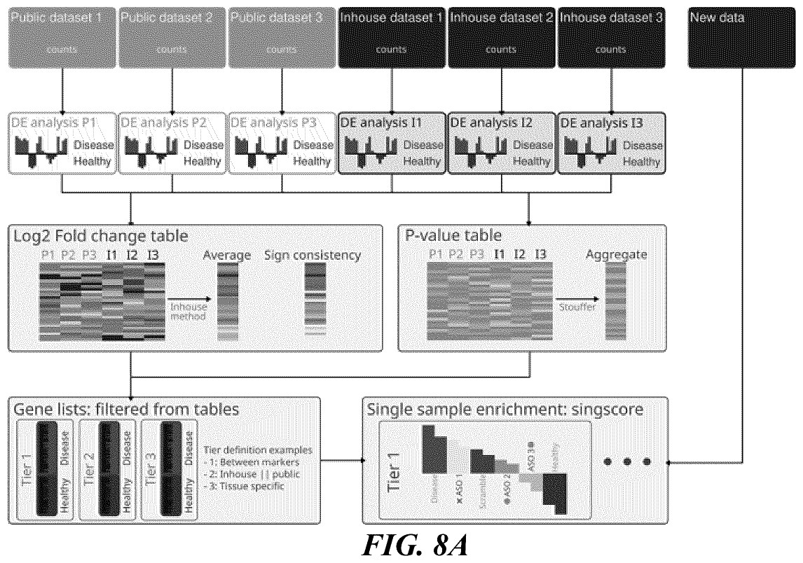

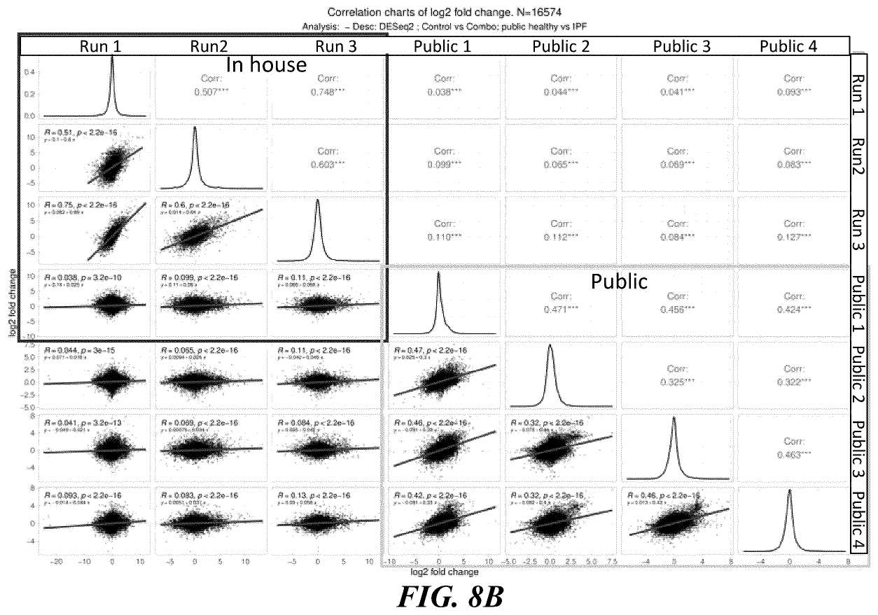

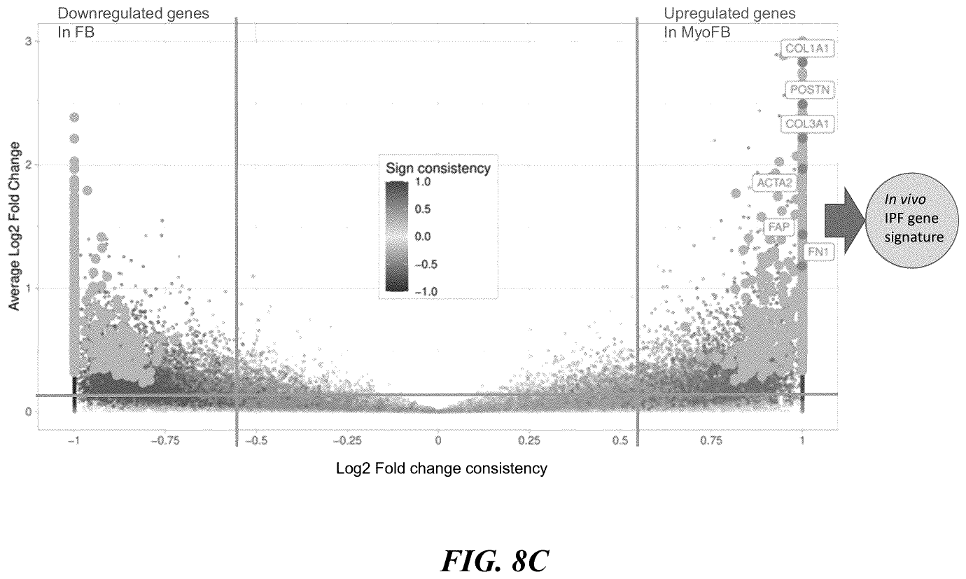

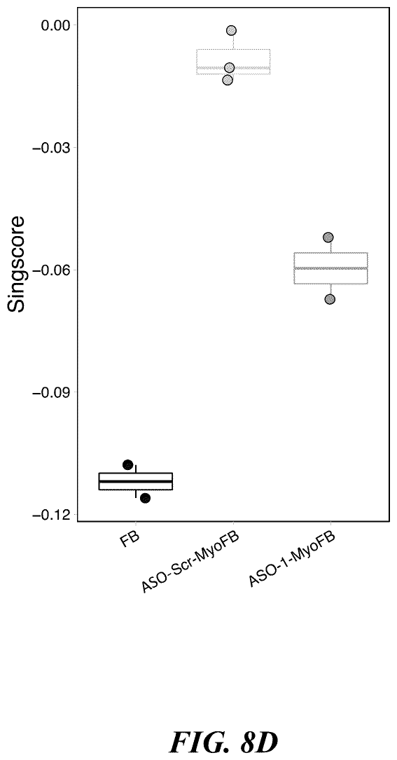

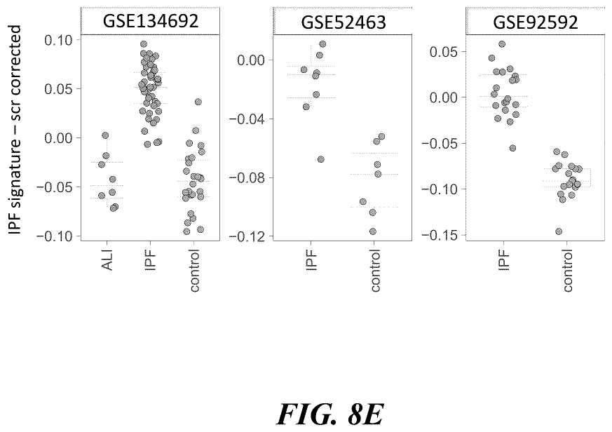

A human IPF gene signature was developed from in vitro and in vivo datasets described herein. This gene signature conveys statistical information regarding how the genes that were assayed which are stably differentially expressed across various in vitro and in vivo conditions behave in other datasets and how the developed gene signature constitutes a core signature of IPF. Development of the human IPF gene signatures used a meta-analysis methodology. FIG. 8A shows a flow chart depicting sources and format of gene expression input data, type of data analysis, and consolidation into a knowledge database of the total results of a method of meta-analysis to build a gene signature for idiopathic pulmonary fibrosis (IPF). The first step in building the IPF gene signature was accumulation and analysis of several datasets of DEG in various lung cells under certain test conditions known from literature. The several datasets include public datasets and in-house developed datasets. The public datasets include RNA-Seq datasets containing in vivo patient biopsies. The in-house developed datasets include in vitro models of pulmonary fibroblasts (FB) and pulmonary myofibroblasts (MyoFB). DEG were assayed and measured for log² fold changes under test conditions and correlation coefficients (R values) were calculated as seen in FIG. 8B. Log² fold changes were averaged and checked for consistency between datasets. P-value tables for DEG were assembled to determine significance of quantifiable differential expression and values within the P-value tables were aggregated between datasets. Next, following analysis of all datasets collected, the information regarding aspects of differential gene expression and interactions among members tested within datasets and categorization within molecular and cellular pathways was distilled into a knowledge database. The knowledge database provides a tool to assess and compare with existing datasets how the core gene signature is expressed in other datasets. Gene lists which are filtered from the log² fold tables and the P-value tables are defined within the knowledge database. The gene lists are grouped into tiers of relatedness and within each tier are segregated into a classification of healthy or disease (IPF) state based on aspects of the differential gene expression. Gene lists are used to calculate a Singscore (a single-sample gene signature scoring method that uses rank-based statistics to analyze the gene expression profile or a sample and also involves normalizing and scaling the data). As seen in FIG. 8C, the genes identified to comprise the in vivo human IPF gene signature form an unbiased group of genes that demonstrated a consistent downregulation of expression in pulmonary fibroblasts and a consistent upregulation of expression in myofibroblasts under test conditions relating to IPF disease state. Pulmonary fibrosis genes of interest including COL1A1, POSTN, COL3A1, ACTA2, FAP, and FN1 were re-identified through this unbiased approach as contributing to the human IPF gene signature as genes which are upregulated in myofibroblasts under test conditions relating to IPF disease state. Data from new experiments testing lung cells under various conditions is then interpreted based on known aspects of molecular and cellular pathways and DEG derived from the knowledge database. Lastly, aspects of the core signature of the disease (e.g., IPF) that relate to specific genes and pathways of the development, and/or progression, and/or severity of a pathological state are tested and validated through gene knockdown experiments using any one of several gene knockdown technologies (e.g., ASO). As seen in FIG. 8D, the effects of ASO-1 treatment using the in vitro model for pulmonary fibrosis in Example 4 including serum starvation and TGFβ treatment in primary NHLFs were tested on expression of the unbiased dataset of the IPF gene signature. ASO-1 treatment significantly reduced the Singscore in representing the IPF gene signature compared to ASO-Scr treatment. These results demonstrate the ASO-1 modulates the transcriptional state of an unbiased selected group of genes representing the IPF disease state to alter a transcriptional state of treated cells in a manner that represents reduced IPF pathology.

The IPF gene signature comprises select genes that are upregulated in the presence of IPF pathology. The IPF gene signature also comprises select genes that are downregulated in the presence of IPF pathology. FIG. 8E shows graphs of three Gene Expression Omnibus (GEO) reference datasets (deposited at NCBI GEO with the GEO accession numbers: GSE134692, GSE52463, GSE92592) and the results of analyzing the RNA-Seq data according to the IPF gene signature. The significant differences seen between IPF and control in FIG. 8E confirm that the meta-analysis methods used to build and define the IPF gene signature are consistent across multiple datasets to distinguish IPF pathological state from a healthy state. These results demonstrate that the IPF gene signature can be used to evaluate human in vitro models testing ASO-mediated transcriptional regulation for test genes of interest and also demonstrate the utility of the IPF gene signature to evaluate new datasets and new genes of interest across species. The IPF gene signature can be used to evaluate effects of modulating expression of a lncRNA on transcribed genes relevant to an extent of pathology, and/or an extent of progression, and/or an extent of severity of IPF.

It shall be understood that different aspects of the disclosure can be appreciated individually, collectively, or in combination with each other. Various aspects of the disclosure described herein may be applied to any of the particular applications disclosed herein. The compositions of matter disclosed herein in the composition section of the present disclosure may be utilized in the method section including methods of use and production disclosed herein, or vice versa.

While preferred aspects of the present disclosure have been shown and described herein, it will be obvious to those skilled in the art that such aspects are provided by way of example only. It is not intended that the disclosure be limited by the specific examples provided within the specification. While the disclosure has been described with reference to the aforementioned specification, the descriptions and illustrations of the aspects herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the aspects of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

What is claimed is:

1. A modulator of a long noncoding transcript, wherein expression of the long-noncoding transcript is associated with initiation, development, or prognosis of pulmonary fibrosis associated with acute respiratory distress syndrome (ARDS).

2. A modulator of a long noncoding transcript, wherein the long-noncoding transcript is transcribed from a genomic region located within chr3:45,806,503 to chr3:45,831,916, and wherein the long noncoding transcript is transcribed using Crick Strand of the genomic region as template.

3. The modulator of claim 1, wherein the long-noncoding transcript is transcribed from a genomic region located within chr3:45,806,503 to chr3:45,831,916.

4. The modulator of claim 2 or 3, wherein the genomic region is LOC107986083 (chr3: 45,817,379-45,827,511).

5. The modulator of any one of preceding claims, wherein the long noncoding transcript comprises at least a portion of XR_001740681.1 (NCBI), locus ENSG00000288720 (Gencode/ENSEMBL), transcript ENST00000682011.1 (Gencode/ENSEMBL), transcript ENST00000684202.1 (Gencode/ENSEMBL), or transcript RP11-852E15.3 (Gencode).

6. The modulator of any one of the preceding claims, wherein the expression of the long noncoding transcript is elevated in a subject affected by pulmonary fibrosis associated with acute respiratory distress syndrome (ARDS).

7. The modulator of claim 6, wherein the elevated expression of the long-noncoding transcript is associated with severity of an ARDS symptom in the subject.

8. The modulator of claim 6 or 7, wherein the long noncoding transcript comprises a single nucleotide polymorphism associated with the ARDS.

9. The modulator of any one of the preceding claims, wherein the modulator modifies expression level and/or an activity of the long noncoding transcript.

10. The modulator of claim 9, wherein the modulator reduces expression level and/or the activity of the long noncoding transcript.

11. The modulator of claim 10, wherein the modulator reduces the elevated expression level and/or activity of long noncoding transcript by at least 50%, at least 60%, at least 70%, at least 80%, at least 90% in a cell or a tissue of the subject, wherein the elevated expression level is associated with initiation, development, or prognosis of ARDS or initiation, development, or prognosis of pulmonary fibrosis associated with ARDS.

12. The modulator of claim 10, wherein the modulator reduces the elevated expression level and/or activity of the long noncoding transcript to a baseline level.

13. The modulator of any one of the preceding claims, wherein the modulator is a nucleic acid editing or modifying moiety.

14. The modulator of claim 13, wherein the nucleic acid editing or modifying moiety targets the genomic region, the long noncoding transcript, or a premature form thereof.

15. The modulator of claim 13 or 14, wherein the nucleic acid editing or modifying moiety is a CRISPR-based moiety, a meganuclease-based moiety, a zinc finger nuclease (ZFN)-based moiety, or a transcription activator-like effector-based nuclease (TALEN)-based moiety.

16. The modulator of any one of claims 1 to 12, wherein the modulator is a synthetic or artificial oligonucleotide.

17. The modulator of claim 16, wherein the synthetic or artificial oligonucleotide comprises a nucleic acid sequence complementary to at least 10, 11, 12, 13, 14, or 15 nucleotides of the long noncoding transcript.

18. The modulator of claim 17, wherein the synthetic or artificial oligonucleotide is a small interfering RNA (siRNA), a microRNA (miRNA), an inhibitory double stranded RNA (dsRNA), a small or short hairpin RNA (shRNA), an antisense oligonucleotide (ASO), a phosphorodiamidate morpholino oligomer (PMO), a piwi-interacting RNA (piRNA), a heterogeneous nuclear RNA (hnRNA), a small nuclear RNA (snRNA), or an enzymatically-prepared siRNA (esiRNA) or the precursors thereof.

19. The modulator of claim 17 or 18, wherein the synthetic oligonucleotide is about 10-50 nucleotides long, about 10-30 nucleotides long, or about 14-20 nucleotides long.

20. The modulator of claim 19, wherein the synthetic oligonucleotide is about 16 nucleotides long.

21. The modulator of any one of claims 17 to 20, wherein the synthetic oligonucleotide comprises one or more sugar modifications, one or more phosphate backbone modifications, one or more base modifications, or any combination thereof.

22. The modulator of claim 21, wherein the one or more sugar modifications are locked nucleic acid (LNA), tricyclo-DNA, 2′-fluoro, 2′-O-methyl, 2′-methoxyethyl (2′-MOE), 2′ cyclic ethyl (cET), UNA, and conformationally restricted nucleoside (CRN), or any combination thereof.

23. The modulator of claim 21, wherein the one or more phosphate backbone modifications comprise phosphorothioate internucleotide linkage, methylphosphonate internucleotide linkage, guanidinopropyl phosphoramidate internucleotide linkage, or any combination thereof.

24. The modulator of claim 21, wherein the one or more base modifications comprise a purine modifications selected from a group consisting of 2,6-diaminopurin, 3-deaza-adenine, 7-deaza-guanine, 8-zaido-adenine, or any combination thereof.

25. The modulator of claim 21, wherein the one or more base modifications comprise a pyrimidine modifications selected from a group consisting of 2-thio-thymidine, 5-carboxamide-uracil, 5-methyl-cytosine, 5-ethynyl-uracil, or any combination thereof.

26. The modulator of any one of claims 17-25, wherein the synthetic oligonucleotide is an ASO, wherein the ASO comprises a gapmer comprising a central region of consecutive DNA nucleotides flanked by a 5′-wing region and 3′-wing region, wherein at least one of 5′-wing region and 3′-wing region comprises a nucleic acid analogue.

27. The modulator of claim 26, wherein the nucleic acid analogue comprises an LNA.

28. The modulator of claim 27, wherein the LNA comprises a beta-D-oxy LNA, an alpha-L-oxy-LNA, a beta-D-amino-LNA, an alpha-L-amino-LNA, a beta-D-thio-LNA, an alpha-L-thio-LNA, a 5′-methyl-LNA, a beta-D-ENA, or an alpha-L-ENA.

29. The modulator of claim 28, wherein the LNA comprises a beta-D-oxy LNA.

30. The modulator of any one of claims 27 to 29, wherein the 5′-wing region comprises at least two LNAs.

31. The modulator of claim 30, wherein the 5′-wing region comprises three consecutive LNAs.

32. The modulator of any one of claims 27 to 31, wherein the 3′-wing region comprises an LNA.

33. The modulator of claim 32, wherein the 3′-wing region comprises two consecutive LNAs.

34. The modulator of any one of claims 17 to 33, wherein the synthetic oligonucleotide comprises at least 9, 10, 11, 12, 13, 14 consecutive nucleotides with no more than 1, 2, 3 mismatches from SEQ ID NOs: 1-4.

35. The modulator of any one of claims 17-34, wherein the synthetic oligonucleotide comprises a nucleic acid sequence at least 80%, at least 85%, at least 90%, at least 95%, at least 96%, at least 97%, at least 98%, at least 99% identical to a sequence selected from SEQ ID NOs: 1-4.

36. A pharmaceutical composition comprising the modulator of any one of claims 1-35 and a pharmaceutically acceptable salt or derivative thereof.

37. A kit comprising the modulator of any one of claims 1-35 or the pharmaceutical composition of claim 36.

38. A modulator comprising an antisense oligonucleotide (ASO), wherein the ASO comprises at least 9, 10, 11, 12, 13, 14 consecutive nucleotides with no more than 1, 2, 3 mismatches from SEQ ID NO: 1 (5′-GGATAATGGTTGGTCA-3′).

39. The modulator of claim 38, wherein the ASO comprises a nucleic acid sequence of 80%, at least 85%, at least 90%, at least 95%, at least 96%, at least 97%, at least 98%, at least 99% identical to SEQ ID NO: 1 (5′-GGATAATGGTTGGTCA-3′).

40. The modulator of claim 38 or 39, wherein the ASO comprises a gapmer comprising a central region of consecutive DNA nucleotides flanked by a 5′-wing region and 3′-wing region, wherein at least one of 5′-wing region and 3′-wing region comprises a nucleic acid analogue.

41. The modulator of claim 40, wherein the nucleic acid analogue comprises a locked nucleic acid (LNA).

42. The modulator of claim 41, wherein the LNA comprises a beta-D-oxy LNA, an alpha-L-oxy-LNA, a beta-D-amino-LNA, an alpha-L-amino-LNA, a beta-D-thio-LNA, an alpha-L-thio-LNA, a 5′-methyl-LNA, a beta-D-ENA, or an alpha-L-ENA.

43. The modulator of claim 41 or 42, wherein the 5′-wing region comprises at least two LNAs.

44. The modulator of claim 43, wherein the 5′-wing region comprises three consecutive LNAs.

45. The modulator of any one of claims 41 to 44, wherein the 3′-wing region comprises an LNA.

46. The modulator of claim 45, wherein the 3′-wing region comprises two consecutive LNAs.

47. A method of modulating a long noncoding transcript in a subject in need thereof, the method comprising administering to the subject the modulator of any one of claims 1-35, 38-46, or a pharmaceutical composition of claim 36.

48. A method of preventing, alleviating, or treating pulmonary fibrosis associated with ARDS in a subject in need thereof, the method comprising administering to the subject an effective amount of the modulator of any one of claims 1-35, 38-46, or a pharmaceutical composition of claim 36.

49. A method of claim 47 or 48, wherein the modulator is expressed or encapsulated in a viral or plasmid vector, a liposome, or a nanoparticle.

50. A method of any one of claims 47 to 49, wherein the administering is performed intratracheally, orally, nasally, intravenously, intraperitoneally, or intramuscularly.

51. A method of any one of claims 47 to 50, wherein the administering is a targeted delivery to a lung tissue of the subject.

52. A method of any one of claims 47 to 51, wherein the administering is in a form of aerosol.
